SEQUENCE LISTING (47 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - DNA, 13361 bp, Homo sapiens
  source 1..13361: mol_type = genomic DNA
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgagaccagggacaaagggaagagtgggctggtgggcgaggcaccttccggctggcgtgggccctctccgggagggggccgagcctctcctgcccgggcctggtcctggcgccagcctcaggcctgcaggtcctaacctcagccactgccagtgtggggttccccattcatccgccttttggagtaggggctgcgctgaggcaggggaatgggagaagtttgaaagggagagagtaaaaggaagccctggcccctgacagcggtggaagtttgtgggcggccaagggaatgtgggcaggagataggcccagggtggggcagatttggcggggaaaagaagggagtgggagtaggaagattagtgctcggggagtccagacggttctgaattctgtccctccggtcagctggctggcctggagggtgttgggccgtggggaggcgaggctgcctgtggaacttggtggagcacaccctgtagggcaggattttggcggctggtgaagtgggggagtgagttgaggagtggggatgggctggtgtggtgggtttgggatgctcatggtgggaggtatttgagaatgggctgggacactggatggggcagggcaacccagtggacagtgtccccagtgccctggccaagccccggcctctcacctggggacattctttacccttttgcctgctgctaggcaggtagccgctgtgggactgagccttcccagggagctagtcctacccccacctggtcagtgtccctgggcctgtcctccagcttcccctccccgctgcttctcacagacctaaacaacaatcccttggtttcttattctacagttcagtttggggaagttggtagaaagttgttttcgtcactggaaaatgtccctttctctggcctcagccttgtttcaatgtatccttgatcgtcctccacgtcttggtccgggaatcatcctgttcagatgtcctgggcccatctagtcaggcagattttccctgccctgcccggcctctgaaggctgcgcctacctcccctctctttagtgccttatactcttcctctcctaccattcctttcttccagcaatctccccagactctcctcagacttctcagagcctctttttttgaaatcttttctcgctaatcctccttcccctcctctctgctccgctctggtcccggccccaggtccccaggcagcacgtctctggtcagggtctcactcttcttcttctgcctcctcctgcctccttagtcccacccgctcttcccttcttcccactgtccttcccccacggtctccccaccagccagctgccctgacatcctgcttctgttttctgtttgggggcggcccctggctccctcacatacctcctgcatgaacaagagcagcttatataacctaaccttccatgccttcgtttctttatctccaaaatgggtgtcacagtcttgacctcatactgttgttttgaagattgaatagactgatacatgttaagtgttcatttgatttattaagtgtgcgctctgggctagacactgtgataggtgctgggattacagcagagaacaaaatccctgcccacagctttgacagtccatcaggggaataggttgtagcaaatagaaagcactcaataaagtttttatattgctgtgactagtagtaattactgggtggctacctgtgttgggaaaacagagggtaaaggtagcctgaacaggtaaagggaagtgcctgcgtcctggggtgcttcagcccaggtgggattatgtctcctaagggacagaagcctggcctggagctggaggaaagggaaaacaaagggaatgcaacatccttctgaatttctcaccattcagtgggcaatgcagagctcacagtgtgtgtgtgtgtgtgtgtgtgtgtgtgtgtgtgagagagagagagagagagagagagaagtggggtaggggagtagggaagaatgatacaggagagactgtggcaaagcaaacaggattttgctgctctcaaagagcttacagcctagtaaccaagatggcttacagtgaaaaatgatttcagagcaatcccgaggaaaatatccacaaatgcattgtgatgtggtgtcctggagcaccagttgggaggaggaggaactggggaaggaggtgagccttagtccactgcctttccttgcttagcaggtctcagctcctgcgctcagctccagaaaattcaggagcttccccacgctgcttcagtgtccttcactgtgcaactgcagcactccctgtatagatctcagtgcctacaactgactgtctttgactcaagtgagagctcttgagagcacgagctgtgtattatccacctcagcatccctagcacccatacgggacctgtcacattaactgtgccccttaactatttgctgaaggaattaaggaacaagagatgtgtcagatgggatggcggagggaaagcctcatagaaaagtggatgtggagctgacatctgaagtcactgcctgtcagggtagctataaaggagggaagcagagttggatactgatgtgaggaagaggagaggaatggagagatgggattttgtgttgatgggcagggtggcaggaagccagacaccttggttcgggagtggaaaaaccatgttgagaaacactaagaaatgtgaatgggagaattagagggagtgggggagaggatggaggaagagtgttgaatatggttccaggtggaggaattcattcattcgtttattcagaagctgttctcctagggcacattctgtgcccagactgtgattagaagtgaggtgaggcatctcagatgggtgctgtggttcatgcctgtaattccagcacttcaggaggccgaggtgtgtggattgcttgagtccaggagttcgagaccagcctgggcaacacagcaaaaccctgtctctacaaaaaatacaaagattagcggggcatggtggggcgtgcttgtcatcccagctattcgggagactgagctcgggaggacggcttgggcccaggaggtggaggttgtagtgagccctgaccacaccactacattccgtcctggtggtgaaggttgcagtgagctatgattgtgccactgcacttcaccctgggtgacagagtgagaccctgtttcaaaaaaaaaaaaaaaaaagtagtgaggcatctgtggaagtcttcagatcatttccatgaccatggaaatgctgtttggagccaggccctggagatggagaggaaggttcacacacttgtgcgtgcaagttaaagcctgaatgaagatttaaaaagtgtgtaggacggatgggagcaggagagaggctagaagacacttgcaataacccaggtgtgaggcaacccaggaatgcggagaggaccgagagatcacagggggaggcctcgcaagatgaactgacacatgggatggcggcagggatagggatggggccctggggagagagcgtggcaagttctcagcattcgtccgggaagtcgatggtgtgtcatttgtctaggtgaggagatggatgaattccgtctggggcatgttaagggtcagggaaatggtcatgtggaagggtgcgcctaccaagctggaggagaggtgctgcaacttctttctgcctttgtatcattcagacacactgtgttcactcatcagtggttctcaaaaggagaggagcacaccagactcttaagtaagggtgtgtgtgcttgtgtgtggggaggtggggggatggtctgaaaactctcccccggagataaatatattcctaccaggggtgctgtctcctcacctccctctttgggaatcactggcttctactagagtggaagacagatgtatcattagatcgatcagttgatccatatttatctgctcccagtctggaggtctggttctgggagctgagaggacaccaggggaggataagacactttctgaccaagacattttttgatctctcatcttataaggttcgtggtcactttggggagatcatatctgtcacccaacataaccatattatgataagagccaaaagtagatagggtcagttcacgtgcttcgagttcacagggactatgggtctaaggagccggggtggaggaaacagacatcgtcaatggtggcttcacgggagggagatgggatctcaactgggcccttggaggagaagctgccacgacctcccccaacaccttgacattaaatgaacagacacatgaatgagggggaaaggaagactaattgggtccctgcaaggtggctggatcggggtcagaccacaaggccgatctcagcgtcgcctccccactctgcagccccagcacaggaagtcacactttaaagcctcctctggcggaaattgtgggggagttggaggggtgttgggccaccccctcaactgtctctccacaggcaccccagcttcctgcccttctgctccaggctggagtctgggcctaaagagctcacctcctgtttctcctgttttgcttcatttacgcaactgctgaggactgggcttactggggccagctggtgccagcagtggtgcccagtggtggggagtctgagggccctggctcctagggatcagagagggctgacctggagcattctgggggccaggggaagcctaggaagcagggctggttcttccatccggcatcccttcttgcctgctccctcgttcctggaagtgggtgttcagggctctggaggctttcctgtattgccagtgggcttggggagggtctgtggagactcagaactggccttgtttcctaaggattgtctggggaccccagggaggcccccaaacccagcacaactggtcagaaccagccaggctgtgggaatgcggtgaacccagggtgggagggcagccttggcttgcttcctgctgggactggggagtgttgggggatggagtgagagctcacggaatgggtttagctgttggagacttgttgaactgggaggaggagctggggcggggcctcagctaaaggccgctgaggggctaggaggagccaagtggccctcagggaagggagggcacagacctgatgggcggaagccagggtcgagggagacttcccttcgggatggaatggggagagggaggcatttcccggaacatgtgggccaagtgggacaagggtctgtggcctggctctttgcatggggaggggatggatgggggttgagtggggatgggaaggagggacttggccataggaagaagggattagatggagtcccacttgcatgcaggctggtgccttctgcctttctgctgactcatgacccttgaggagctggggaagctgctagttccctctcccctccctaggtctccctccctctggcctgagtcactggggcggagttgctgggaaaagatttccctttcccggatctgacttaacccccagagtgctggaaagagaagggaacacgtggcctgagaaagcctctctccctccctccctccagggaggctcatcccccactggccagaggtccctgaaaagctccctttaaggctgtctggggctggcgtcccccagttcttcatcatgactctgcctcaagccccctggatgggattcaaagtaccagtgaccttaggtgctccagtggcttcttcggggaaaggaaccacactttcaggactgggaagttcttcccatcaccaccccaaacccttcctgttgccctggaagccccagtcctgttctcagcagaggtggcacggtgttggctggtgcgggcaggggaaggttgttgtcctctgagcaggggcacacgcctccacctgcgggggctgctgttgtgtttctgtgtgtggcttcccctgtttgcggctgaggcttgaacttccgggcctgcacagcttacagctgcagcgtctccccgtggctgactcagggtgactggcctcctgctccgaaatgtggagttggtgaggctgggtggctgtgggctgcctgaccctccttccctgccctagggtttctgtgatctggtgagtcagttgctccccagtgtttaacagacattgaggacaccctcttatctttacacaaagtgtctcttatagtagaaaaaaaaaatgaagcccagggaaaaccagaaatgaagctggcagagatcaaagtccaagttagagctaaatattcactcctggctttgctttcctggcactgatgccggaacaggacaagccatttagctgctgtggggttggcctgagactgcaaagcacaccttccagaatgccatggtgtgcagggggctccaggactccccagcacgccctcagctctgacctgacagtcatccaagctgggtcgctagccttggccagctctatttgcctatgtcctgcacacctttgcccactcctgcccccgtctcaactttgtcccccgtctacccatgcaggatccccaacctttcccttttactctcctccccatttgtccttgccaaccccgggtgtttgtaaattttgaggtggaggggatgggccagggaatgtgagggcggaggcagattgaggtttgatacaaacatgtaaataaacttccttcttctgtccactccccaggagtggtgctcacgggaacatcactcgcccccaccgccagctgactttttcagaaagcttttcatggtgtaacatattcctgggatgtgcatagatcctcattgtttacctctgtgaatgttcgcaaagcgatcacacggtgaacccagcacccagatggagaaacaccgccccaatctttagggctgcttgttggaagaaggggccatcactgaagtaacctgccaattcccaatcaaaaacacatcctttcaacatctgccctgtgtccagcactgttagctgctgtgggggatttcacagtaaggataaaatacagggctgggctcacgcctgtaatcctagcactttgggaagccaaggtgggaggatcacttgagcccaggattttgagaccagtctgagcaacgtaacaagaccctgcctctactaaaaataaaaaaaaattagctgggcatggtggttcacggccgtagtcccagctattcaggaggctaaggtgggaggactgcttgagcgtgggtggtggagggtgcagtgattgcatcactgcactccagcctggacaacagagcaagatcctgcctaaaaaaaaaaaaatacagcttagatctggggcctactagctttgagttgagggaacaaaaatgaacacacaggacaactagagaacaattaagcatcagattgtatggccccaactgtctaagtttcaaggaagaactctaaacttagtgagtggcgtggcctgggcggaatgtttcactgaggaaggacttgagccagggaagttttagatctgctacccctaagcttcccatccctccctctcttgatggtgtctcctctatctgattcttccccaggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggtaggggcaagtggaaacggtgaatgccctcaggtctggggtgctgcttctttctctgcttcttccagttgttcttccctaactttgctgtctctcctgggctgggattttctccctccctcctctcctagagacttcagggaatcggccctggctgttgtccctagcatggggctccttccttgtgttctcacccgcagcctaactctgcggccccattcacaggaacctacttgtacaatgactgtccaggcccggggcaggatacggactgcagggagtgtgagagcggctccttcaccgcttcagaaaaccacctcagacactgcctcagctgctccaaatgccgaaagggtgagtgtgcacaggcaggagagtcaggcgggtcttgagtggtgtgtgggtgcctgtctatgtgcaggctggtgggtgtgggcaggaaggtgtgtgttttggtgggacactgcatggatgtgagtgtgtattacagagacacacacttaggggtatgtcaggaaggggatgcagggacaggaggatgcaggactcataccccatcttctcccctcaccagaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgtgagcgcagctctcctgaggccaagccctctccccaccccaggggttggccccttccccatgcggtggcacttcctttccttccccctcctgtattctgtgggtctgacaaccaactcctctctggccgcccccaccctgtccctcgtcacttcctctgtcctgtggggtgggggtgcaggcgcttctcctttagctgtgccgcacttctccctacaggccaggagaaacagaacaccgtgtgcacctgccatgcaggtttctttctaagagaaaacgagtgtgtctcctgtagtaagtgagtatctctgagagctgctgggcactggatggtggcatgggttgggacgggtgactggtgggaaccattagctgggcaacagatgccaggatgccccagagtgctcagggtcctactggctgagtaggagacacttcgttaagacaccaggcagtccttccccttgctcttcaaatctgaagaagtctttgaggatggaagatcatgccccaagggctggcagcccttccaactcagatatgtagattcttggatctacgatagctcattggttctaggacatacactcttatagctctgaaatcaaacctcctataactggtgactcatcatggttgaattggcagctctgtttgcgtctgggtagtaatgtaaagaaaagtgccttttattcttgatggcgtcttaggtttgatgcaatatggtatttcctcattagtcactgtccaggcctccttactcctggctccacagaggctgttcttgtcactcacttgcaaagaataaactctgagggctctcagagtttgaaccccagcatagccacttactggctatgtgacgttgggcaagtttcttaacatctctgagcctgacttttcttttggtgttttttttttttttttttttttgagacagggtttcactctgtcacccaggctggagtgcagtggtgcaaccgtggctcagcctccacctccagggctcaagccatcctcttgccttagcctcctgagtagctgggattagaggcacacaccactacacccagctaatgttttactttttgtagagacagggtcctactatattgcccaggctggcctcggactcctgggctcaagcgatcttccgcctcagcctcccaaagtgctaggattacgggcatgagccaccacgcctggcctgggccttagatttcttatatttaaagtaagcataatgacattcatttggtgaatttgtgagaaccaaaaacaaagaaacaaacaaaacctacaacacgtctgacacaaaactatttattttccattaatcttcttttttttttttttttttttttttgacacagagtcctgctctgtcgcccaggctggaatgcagtggcgcgatctcggctcactgcaacctctgcctcccagattcaagcaattctcctgcttcagcctcccaagtagctgggattacaggcacgtgccaccatgcctggctaatttttgtatttttagtagagatggggtttcaccatcttggtcaggctggtctcaaactcctggtgatccacctgcctctgcctcccaaagtgctgggattacagccgtgagccactgcacccagccggcttcatctcttcttgaaatcacttttataccattctatgtggttctcaccatgagcttgagtggtgggctaaagtgcctctccctgctttcagcttcctgctgggaactcactctctcaagttccttccagcaccaccccatagagttcccatcactccacactgtccagtgacaactcccaacatggaagatctgctagttctacagggtgctctctggctgccccagtaacatgtgtttttaaatttttcacatgcatgtttgaccccgactccccgaagtcaggtactgtaactagcagtgtcatttaagaaaaagccctttaacctctctttgccaaaggattcttatcagcaaaacagtgatgaaacaacaatcccataacagctagctggctaccttctcaagcacttattaaatgaggcataatgattttgcttaatcctcaatcctgagaggtgggcgatccctgtggtgatgaggaaaccgaggcttgggggttaatggcttgcctagattcacactgctagccaaggaatgaactggaatttacaccctgaccctgactgcttttcacattttctacacagccttttcaagatccctgccaattctaaaattaaatgattctatgattaactgtgtttcattcttctgcatcagttcccaaaacaaattatatcaagagacagcaaaaatatttgtaaagaaaggatgtccaacaatctgtgtggttgtttttctgtgttcctccaatggtagggcctctgttcaccagtgccgtctcttcttttagctgtaagaaaagcctggagtgcacgaagttgtgcctaccccagattgagaatgttaagggcactgaggactcaggtgaggagaagtgacctggtgcccatgctcacctgccctctccctcttcttgcccccacccgtccatccatcccacccatccatctatccctgcggcccccctctgcccgctcctctgaccaacacctgctttgtctgcaggcaccacagtgctgttgcccctggtcattttctttggtctttgccttttatccctcctcttcattggtttaatgtatcgctaccaacggtggaagtccaagctctactccattggtgagtgggggctttgggagggagagggagctggtgggggtgagggaggacatgggtgggtgcgatggacatgtgtggagggaggtgaggagtgtcccctcagttcataccgctggggactctgggcagaaggtggccctggatggctggggagatgtcgagctgcatcagtagctctctcgtccctggggccacataggccctgaggcatgtcaccacaagtccccactgccagctgagtccagggtgccagggctgagagaggaagtgaaatttatgatgctttctttctttttcctcagtttgtgggaaatcgacacctgaaaaagaggtgagatgaaatgagagagttactcccaaatgtccctgaccattccttataattgcctaatgctcagatcccctggaatcatccttcactttccgggggctcgcctcattccctctaagtcccaacccccacgtagaataaagagggccggggctggttttcgctgccgcactaatgttgcgccaccttctctctttcagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacagtgagtttctcccgcggctggagacgaggaggctgggggagggccgggggagcgcgggaggcgctcccagaggggaccacgagaggcggagggcgcgggatgcggggcggggcctggggttgccgcccgaggctcaccggcccgcgtccccgcagctgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttgga

SEQ ID NO: 3 - DNA, 2258 bp, Homo sapiens
  source 1..2258: mol_type = genomic DNA
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaaaa

SEQ ID NO: 4 - DNA, 2102 bp, Homo sapiens
  source 1..2102: mol_type = genomic DNA
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaa

SEQ ID NO: 5 - DNA, 2374 bp, Homo sapiens
  source 1..2374: mol_type = genomic DNA
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaaagtcctgctctgtcgcccaggctggaatgcagtggcgcgatctcggctcactgcaacctctgcctcccagattcaagcaattctcctgcttcagcctcccaagtagctgggattacagctgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaa

SEQ ID NO: 6 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 7 - protein, 347 aa, Homo sapiens
MGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 8 - protein, 302 aa, Homo sapiens
MQWRDLGSLQPLPPRFKQFSCFSLPSSWDYSCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 9 - DNA, 12761 bp, Mus musculus
  source 1..12761: mol_type = genomic DNA
cgctcttgcaacaccacccccgccactctcccttcccctcctaccttctctctcccctcagcttaaattttctccgagttttccgaactctggctcatgatcgggcctactgggtgcgaggtcctggaggaccgtaccctgatctctatctgcctctgactttcagcttctcgaactcgaggcccaggctgccatcgcccgggccacctggtccgatcatcttacttcattcacgagcgttgtcaattgctgccctgtccccagccccaatgggggagtgagaggccactgccggccggacatgggtctccccaccgtgcctggcctgctgctgtcactggtgagatgggagactggaggggagggtgggttgctagcgaggcgccgggcaggagtgggcttgtactaggggtagagatgggggcaggggcttctgtgctttagtttgacgccaggtttgctgtgcctcacctggggctgccctgagatgtggaagggagaaatctgagagggagagaatctggcaagccaagctggcaagcctgggcccctgacagtcaaggtcctggcccgtgttgggctgtggtcaggccagctgctcggccagtggagttagacggagaagccttctttctctggccgaggttgaacagcgtggggcagggtgttggaagactgggtgctaggagaaccgctttggcgggacgagtttgggaaagcgtgcttgggagtggacccgaaactggacaaagctgggcagccttccggtcccagacttctccctgagacagcctctgaccttctgccgcctccatttctgcttccagacaagaggtccctcctggcgtgaccctccttaagtagctaatacctctttatctaggccatgtatcctcctgctccctccccacagacccaaaagtcagtctcacggtttccaattccacagttccaattggggaagttggtagcagcttgttttcgtcactggaaaatgtacttttctctggcctcggcctagtgttcatgttctcttgatttctgtgtgtccataattattctgtgtagatcctgggggctccctggctgtctgggtgggctgtaccatcggcgcagtgttctctgggtcctgcctctccctggcccagcgttatccctgagacctgcctttttctccctcccctttcactcctcattctcttctggtcagtctccagagcctccttgcgggggtggggtgggggggtgggggtggctcctttgtgtctattcccttccctttctctacacaccattctaaagcagtggtcctagaaccctgcctgggtgtcacctcccttctcctggtcctgtcctgtctcccagtctctcctgggagacttgtcaaggctgcccctctggctgttctccaactcccccacccacttaattcttttctctttaggggtagatcacctggatttagggctcagcccagcacccttaccctcatgaacgcaggcatgtgatgtgactcccccgctttttgtttcttttatttccaaaatgtctacaatgatcagtacctcatagttgttattaagattgagtagatggatttgtgttaaatatttattcatttccttccctgctaggtccaattcagattaggtgaatgatcccaaaggggagtaagaagtatgccacaaacagaaaggactcagcttggcttctctcttctcatctctagcagtgatactcgtcactactcacgtcactactcattgtgacggtagatgtcacaataactgtcagagtgcagtggcccagctaggtagaggagagaggctgagccagttgtggaggcccaggtgagggtgggggcaccgtgaacagaagcagatcctgaagacaaggcacacggaaggctactgaagtctacatggggctggccactgccttatcagtacagagcacagtgcctgtgaggtgttcaggtaggacgaggtgctgaggtcatgaggcagtgatggccgtggcagggatgtggcaaggccaagaagactttgctatttttaaaaagtttcagtacaggagtggaaatgactttagaatactctggaggaaaagacgttccaaggccttggtgatgtactatcccagaggcagaactgggggaggagggtgggctatatgccaactgtcttcttgcttaatggcctttgcctgtctgctgggctgagcttgcctgcatagaccctaaggctcatagtgtgttatcatctttggctcatggagagctcttgaaactgtgagctatgtcccatccccagcacctatccaggccctgaaaaagaaaccttgaccgttgactaatggcttaatcaattcaggaacaagagattggtcagatgggaacaaggcgggtgttgcatagaaaggaggacttggaactggtgtgcagaagccaggaggaggaagaacagaggagttgggcgcctgggttgggggaatggagaggtgggactttgaagtcacagaagctgaacatctcagctcggggtggagtggccatgttgagaaatgtgacagagagagaagggcactctggcgtttgtgaaaaagaaccttgagccagtcaccttggggcatgcctgttatcctggaacttgggagatggaggccagtgctcagatcgaggcccagttttggctatatagggagctggaggccagcctggatgactgagactgtgtcttaaaaacaccaaaaagaaataaagagtcatgcttaaggacctaggtgggggcctgttttcctctttccctccctctctccctcccttcctccctccctccctccctcctccctccccttcctcctttgctatccaccaagcagtctggccagactgtgttccaaagaacagtgagtttctcagatgtatgtgctcatggaaggatttatccatggaaatgatgtttgaagccaggccctggaggtgcagaggaagattcacacccaggtggtacaagttgaagcctgaatgtagataagaaactatgtaggtagcctcagcccctgcaaaggtttgaggcaggcacatcacttgaaccagaaggttcgtggcagcctgagccacatagtgaagccctgagacaccactgtggagtaggaaggagaaaggcagttgtaaaaaccagggctccgggtgtggggtaaagggtggagaggttccagaaggactgactgatgagtcttagggctgttgcagagtttgcagcattggtaccaagaagtcggtggtgtgacatctaggcaaggagcttggtgagctcagctgggggcaggttaaagtaggtcactggctggctaagtggaagtgtggttgtgctgtctggagcagcccttctcagcctttcttccagctaattccagctgcatggctcgtcagctggactctgcaggagatgagcaaatgtggcagaattgtcaccagggacatgtgcacgagtgtgcccgtgtgtggggaaggggatgcacagcttctaaaatacctcccagaggcagagtgtgtttgaggcagggggatgctgcttccttccatccggctccctgactggaaggcacctcagtccctcaggtctgcaggcctggctctgggtgatgtgaccaagacatgctctgatctcttaactcagagggtttgtggtctgtctggggagattgtatctactcatttgaaataactatagcttctgataagagcccagagtagatatggatagttcgtgttattcaaccagaacaggcggctaggtatccgaggagcttgggtggggagacatgaatcatcgacggggggcttcgtgggtggaaggtaggattgcggcagcgtggcagcgctctctgaggaggtcacaccgaagagctcctcaccaccgtaggtcttgacattaagtggaacacagtttaacctaatgtgcacgctgatgtggggcttagtgagtgggtccccaaaaggtggctgggtcaagtttgaccacagggtccatttcatcttcgctcccctactcagcagccccggcacaggaagtcacactttaaggcctctctaggcggaagctgaggagtagttagagaggccttgccacctcctcagccatctctcctctcctcaggcaccccccagcatcctgcccctcctctgctccaggcattcaagtttgggccaggacctgtgtcccttgtcccttgttggcctcacaatgcaactggtaatagggtgcctattgaaagagccagagggccctggaatccctggagcatgtgagatgagagagctggctctgcacctgctccttctcttttgttaccttgtttttgttgagagtagacctggctgtcctagaactcacattgtagagcaggctggccttgaacccagggatctgcctgcctccacctctcagagtgcctggattaatgtctagcttcctccatctcttctaaccttaagagggctgtgatttttgttccatagctacttgacctggggagaactggccctgtctcttaaaggttgtctagggagcatccaaacccaatgcaagtggccagaaccaggcaggctatgggaatgtggtgaactagggcgggagggcaccctggatttgattcttgttgggattggggagtgtcaggggctgccaaaggcccagggagccagggagcagggatcctgggaaaggctttatcagctgaagacgatgttgtttccctttgaggagggatcaaggcagctccaagtccaaaggaaggatgtcagagatggaggaggtagatcctctgggaggagcttactggcctgacagggaagccaggggtcagaaagacttccctttgggatgggatggaagagggaggcattttcccgaaatctgagcccagtgggacaggggttcattgcctggacgaatggtctgagtgggggtgggtgagagagagagagagactgtgcacccaggagggagcagatggggttccacttgctgctggttggcttttctgctgattcacagcccttgttgagccgggaagctgcagttccctgtccactgtcctagatctccctccctggcctgagtcactggggcggagctgctgggaaaagatttccctttcccggatttgacttaacccccagagttctgcaaaggaaggacacgtgaccaaagaaacaggctccttctctggaggctcgtccatcgctggttgagactcgcccccacccccacccccgcaaggccgttcttggcctggagcccacatctacctcttcctgacactgcctgatctgttggtttggcttcaggttctctgatggggttggaagtaccactgaccttaggtgctccaagcatttcttcttcggggaaaggaaccacactttcatgattgggaagttcttatcataactaacccttcctgtcaccctggaagcctctgtgtgtcgtgaggggtgggggtgcacccctgcctgagagattgctggtgtgctttctgtgtgtggcttcttgggtctatggctgaggcaaggggcttcctggcccgtgcagctgctgtgccgaggaggtagcacttctagtaacagcagctgacagcagggtgcaagctgccagcctctttccaaacggagctttgggggttgcagagcccccaaaggcagctgtgagtctaggtgttaggtctctcctgaatgtgatctgattggtcagttgcttctgcatctgtcttgaagacctccgctatcttgacgtaacaacgctatgcgagggggggaggggagatgaaagaaggaaggctgagggaaacccaaaaccgggagagatcaaagagcaaggtctacattgaaactaagagggctttgcgctctccatactgaagctgtttacatacaagaaagccagttggttgctatgagctttgccaagattataaagccagccctctagaatgccctgctttgggactcctggcacatcctcagctcctgaattgagggtcatcttgaatcagatcaccaatctctggtcaactctagtacatgacacatttgtctttggtcttacttaatccttgtcgcgcctgcatgctcggagttcttccttttgtttctgtcccgcaatggtgcttgggagagcattatccccaggtgcgcattcatatctgggtgtttgagtgtgcacggaggtcagaggtcatcctcaggtgtttgcttcccatcagttggactaggttgcttagacagagatcatcaggggcctacctggtgggctcccctccatcaccgggaatacaagcccgtacctggctttttatgtaggtgatggtgctcgatgtcaggtcctcacgtcgcacagctgacactttcctgaaccatcttcatgacagccctcccttcaacttgatttcccccccccagtagtactttttgaattactgtcatggaatccaaagaggtttgtagatcctaatggctcacctctaggaagtgtccacaaacaggcttagtgtgaaacccgtacccagatatagaaatgccaaatgagtagtaggattgattgtctgttggaaaaacaggctgtaacggaaacagcctgctacttctggctaacatttcttggtcgggcagagggcattgcagtgcttctgtgtgagacctggagctccaccctcagccctgcagacacacggggaaactaaacattccttgaccggctctcttgtgatcagcactgggagccgcttcagggaactgaacgggataaaatgcagctggggcctgagacctaattgctcagttgtagaaacaaatgctagcatacagagccaccagagaccaagaaaatactagactgcacagtcgccaccatctaggggagagtgtccctagggggtggggggaatgtgaacttgtctctctctctctctctctctctctctctctctctctctctgtgtgtgtgtgtgtgtgtgtgtgtgtgtgtgtgtgtgttcaatttgttgctacctctaaggccccacccctgacagtatttcctctttctgattgttccctcaggtgctcctggctctgctgatggggatacatccatcaggggtcactggactagtcccttctcttggtgaccgggagaagagggatagcttgtgtccccaaggaaagtatgtccattctaagaacaattccatctgctgcaccaagtgccacaaaggtaggagaaagtgagagcagagaattgtcttgagatttcttttgctttttttttttttttttttttgagacagggtttctctatacccctgtctgtcctggaactcactttgtagaccaggctggcctcgaacacagaaatctgcctgcctctgcctcccgagtgctgggattaaaggcgtgcgcctccacgccccggcttcttttgcttgtttctgatgttccccgaccttggtccctgggagtccccttattttgctgccccactccctgcccccgacttgggttttgccctttagttctccactacagcctgaccctaaccctatctacaggaacctacttggtgagtgactgtccgagcccagggcgggatacagtctgcagggagtgtgaaaagggcacctttacggcttcccagaattacctcaggcagtgtctcagttgcaagacatgtcggaaaggtaagccttgggattgggccagggctatagaaggtgcatggtgtgtgaagacgtgcgaacatgtgtgtgtgtccgtgggtgttggccaggaggtcaggatttcgaatctgctcgtgagtgtggcggtagtatgcatgcgtgcacatgcaagctcgggcctgtgtgcgtaggaggagtgtctgttacaaagacgaatgccatgtggcagagccagggggcgtcaagatttgtgtgggaaaagggatgtgagactcacacaccatttccttccctcttcagaaatgtcccaggtggagatctctccttgccaagctgacaaggacacggtgtgtggctgtaaggagaaccagttccaacgctacctgagtgagacacacttccagtgcgtggactgcagcccctgcttcaacggcaccgtgacaatcccctgtgagcaccggtcacccaagtcctctcccctccccgggagcaccctgcccccctcctctgccccccaccctatcctgtctgtgtgtctgagctcctatgtggcacctgcattcccgccctcgtccctccctcgggccctcagactgcaggcacgcgctcttagtcttaaccgctttcccacaggtaaggagactcagaacaccgtgtgtaactgccatgcagggttctttctgagagaaagtgagtgcgtcccttgcagccagtgagtatctgccaagtgcttggggttcaggggtgggtgagcagctgtggctgaggggccccagcagggtcaaaggtgggatcctacagccggatgagcagaagacactacaagacatcagacagccctttccctttctccaggacgttaggggataagcaatagggcttagcgggtaaagcgcttgcagcaaacgtctggcaacctgagttcaagctctggaaccccgtcttgtaaaggtgacaggagagaatatcaggtgcacaacgtcatcctctgactgccacacacacgtgctatagctcgcatgcctcttcagtgctgggcagttagatccgagcgcgctgttcttgactgcactctggtgcatcttactgttgatggtatcttaggtttggctcgcacaagctctaccgctggggatcccttcttcccaggctccctgacagacttcctcattcctgtggctccgcatgggctgctcttatacatttgcttccagagaagactctcatagttgacctgacttgggggttaaatcccaacttacctttttcctggctctgcaaccttgggcaagttcttacctctctacgccttatgtttcttaacctttagggtaaggagagtgggggtggagatgtggatcaacggtagagcactggcctagcgtattggaggctctgggttagatccccaaaacagacagctaaaccaacaatctataggactgtctgaaccaaagctgtctactttctatttttttttccacgcagtttcatctcaaaagagctcatgtgcccatgtgttcaccctgtcatgaggtagacaaaacctcctttccctgttctgctgagaacgcatgttcctctgccgtccccctgcccccacccagtgtagttttctcatcccctctgccctgtgacagctctcaatggctgccccaatgagagacatcctaaaacctcccgtgcccttgaggcctgccgtctccaaagtcaagtgctgtaagcagccggaccattggctgggaagccctccagcgtctcatatgagtcctctttctgggcacgcggcttcatcagcttaaagcaaagaccggctggtttcattcagctcctggtggttggcgttgctgctttagagtcagtgctgaggcaggggcatcatgatcagagcatactgtgcaagcacagtagctcacctcgcagcccctgagaggccaaaggaaagggtgtggagaggggtcaaaagtgagccctcagggacctaacttatactagtccccatgtcccagtatgctgggaccaagccttcaacacatggacccagcaggacatttcagatccatgccctaccggcaaacacaatgataaacccagtctactacagctgccgtctaccgtgccatgtcagggccaagtacttgatcccagaaggaggtgtcatgagcgccatgagatgctggaccagaaaagtgaagtgacttatttctgtcatgttgccagtcaatgactggaatggaatttttactcttgactataaaggccttttaaattttatttttaaaatactgattgattgttgaatccgggctcttcgcatgctattcacgtgctctaccatggagccaaaataccctcagcccttcgaccgtttgattttatttcatttcattttatttatttctttttactcagggtctcactgagtagccttggctggcctgggactaggtggaccaggctggccttgaactcctaaatatctgcctgctttagcctcccgaccactgggatgtgtcaccatgcctggcatttaaaaaatactttaagaattatttattttcattctatgtgtatgagtgttttgccagcatgcatatatgtgtactgtgtacattgctcagtatcctcagaggccagaagagggcgttagataccctggaactggagttacagatggccgtgagctgccatgtggggtgatgactgtgtcaaactcatcaagatggccgacttgccctctctctccctttcccctccctttcctctctagtctctttctggacaggatctcctgttgcccaggctgaccctgaccttgctgggtagccatgggtggccttaaaccgatcctttcgtcacttcctgccaagtgctgagatcacgtgcatccacttctgctcctgcctgtccacacagccttcccgggctcccatcagcttgtaaactgttgtacggtgtggactgtctcgttcttccccaccagctctcaaggcgggttgaagagatagcaaaaccatttgcaaagcaaagaactgcaggcagccttcctgctaaccggtgccttctcttctttcagctgcaagaaaaatgaggagtgtatgaagttgtgcctacctcctccgcttgcaaatgtcacaaacccccaggactcaggtgagggggggtggcttggtgccagctcaccgagctggctggctgactccatgacctccctccacccccaccctttaccccccagccccctagtctctgctgtggcctcacactgagcaacctctctggtctgcaggtactgcggtgctgttgcccctggttatcttgctaggtctttgccttctatcctttatcttcatcagtttaatgtgccgatatccccggtggaggcccgaagtctactccatcagtaagtaggactttggggatatagggtgttggtggagatacgggaggggttcgggtgggcaagttggagggaaatgaggggtggcctctcttcatggttaaggactctaggctgagtggccctggatggtgggagggaagccatactgatatccgtaattctcttgtccctggggccacatagaccctggggcatgacaccacaagtccccaaggtcagcgttgaaagggaagtgaaattcatgacaccttgtttctttgttctccagtttgtagggatcccgtgcctgtcaaagaggtgagaagggacgactccagcttccctgactactccttccaacgcctgatacctaaattacttggaatgagggcggtgggctcccttagctcctctgtccccacccccataaagaataaagcgcccgaagggagtccttttctttactcccactaatgctatgctttctttctttcaggagaaggctggaaagcccctaactccagccccctccccagccttcagccccacctccggcttcaaccccactctgggcttcagcaccccaggctttagttctcctgtctccagtacccccatcagccccatcttcggtcctagtaactggcacttcatgccacctgtcagtgaggtagtcccaacccagggagctgaccctctgctctacgaatcactctgctccgtgccagcccccacctctgttcagaaatgggaagactccgcccacccgcaacgtcctgacagtgagttgggttcctgctggggcattactcctggagggagtcgggcagggtggagggcacaaaggcaggctgctgctggaagtgcacggtccttgtccccagatgcagaccttgcgattctgtatgctgtggtggatggcgtgcctccagcgcgctggaaggagttcatgcgtttcatggggctgagcgagcacgagatcgagaggctggagatgcagaacgggcgctgcctgcgcgaggctcagtacagcatgctggaagcctggcggcgccgcacgccgcgccacgaggacacgctggaagtagtgggcctcgtgctttccaagatgaacctggctgggtgcctggagaatatcctcgaggctctgagaaatcccgccccctcgtccacgacccgcctcccgcgataaagccacacccacaaccttaggaagagggacttgaacttcaaggaccattctgctagatgccctactccctgtgggtgaaaagtgggcaaaggtctctaaggggaaggctcgagctggtagccacttccttggtgctaccaacttggtgtacatagcttttctcagccgccgaggactgcctgagccagccacttgtgagtggcagggagatgtaccatcagctcctggccagctgagggtgccaaagacaggattgtagaggaaaggcacaatgtatctggtgcccacttgggatgcacagggcccaagccaagcttctcagggcctcctcagtgggtttctgggcctttttcacttttgataagcaatctttgtatcaattatatcacactaatggatgaactgtgtaaggtaaggacaagcatagaaaggcggggtctccagctggagccctcgactcttgtaaatacactaaacgtctaaaaatga

SEQ ID NO: 10 - DNA, 2186 bp, Mus musculus
  source 1..2186: mol_type = genomic DNA
cgctcttgcaacaccacccccgccactctcccttcccctcctaccttctctctcccctcagcttaaattttctccgagttttccgaactctggctcatgatcgggcctactgggtgcgaggtcctggaggaccgtaccctgatctctatctgcctctgactttcagcttctcgaactcgaggcccaggctgccatcgcccgggccacctggtccgatcatcttacttcattcacgagcgttgtcaattgctgccctgtccccagccccaatgggggagtgagaggccactgccggccggacatgggtctccccaccgtgcctggcctgctgctgtcactggtgctcctggctctgctgatggggatacatccatcaggggtcactggactagtcccttctcttggtgaccgggagaagagggatagcttgtgtccccaaggaaagtatgtccattctaagaacaattccatctgctgcaccaagtgccacaaaggaacctacttggtgagtgactgtccgagcccagggcgggatacagtctgcagggagtgtgaaaagggcacctttacggcttcccagaattacctcaggcagtgtctcagttgcaagacatgtcggaaagaaatgtcccaggtggagatctctccttgccaagctgacaaggacacggtgtgtggctgtaaggagaaccagttccaacgctacctgagtgagacacacttccagtgcgtggactgcagcccctgcttcaacggcaccgtgacaatcccctgtaaggagactcagaacaccgtgtgtaactgccatgcagggttctttctgagagaaagtgagtgcgtcccttgcagccactgcaagaaaaatgaggagtgtatgaagttgtgcctacctcctccgcttgcaaatgtcacaaacccccaggactcaggtactgcggtgctgttgcccctggttatcttgctaggtctttgccttctatcctttatcttcatcagtttaatgtgccgatatccccggtggaggcccgaagtctactccatcatttgtagggatcccgtgcctgtcaaagaggagaaggctggaaagcccctaactccagccccctccccagccttcagccccacctccggcttcaaccccactctgggcttcagcaccccaggctttagttctcctgtctccagtacccccatcagccccatcttcggtcctagtaactggcacttcatgccacctgtcagtgaggtagtcccaacccagggagctgaccctctgctctacgaatcactctgctccgtgccagcccccacctctgttcagaaatgggaagactccgcccacccgcaacgtcctgacaatgcagaccttgcgattctgtatgctgtggtggatggcgtgcctccagcgcgctggaaggagttcatgcgtttcatggggctgagcgagcacgagatcgagaggctggagatgcagaacgggcgctgcctgcgcgaggctcagtacagcatgctggaagcctggcggcgccgcacgccgcgccacgaggacacgctggaagtagtgggcctcgtgctttccaagatgaacctggctgggtgcctggagaatatcctcgaggctctgagaaatcccgccccctcgtccacgacccgcctcccgcgataaagccacacccacaaccttaggaagagggacttgaacttcaaggaccattctgctagatgccctactccctgtgggtgaaaagtgggcaaaggtctctaaggggaaggctcgagctggtagccacttccttggtgctaccaacttggtgtacatagcttttctcagccgccgaggactgcctgagccagccacttgtgagtggcagggagatgtaccatcagctcctggccagctgagggtgccaaagacaggattgtagaggaaaggcacaatgtatctggtgcccacttgggatgcacagggcccaagccaagcttctcagggcctcctcagtgggtttctgggcctttttcacttttgataagcaatctttgtatcaattatatcacactaatggatgaactgtgtaaggtaaggacaagcatagaaaggcggggtctccagctggagccctcgactcttgtaaatacactaaacgtctaaaaatgaaaaaaaaaaaaaaaaaaaaaaaaaaaaaaaa

SEQ ID NO: 11 - protein, 454 aa, Mus musculus
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTAVLLPLVILLGLCLLSFIFISLMCRYPRWRPEVYSIICRDPVPVKEEKAGKPLTPAPSPAFSPTSGFNPTLGFSTPGFSSPVSSTPISPIFGPSNWHFMPPVSEVVPTQGADPLLYESLCSVPAPTSVQKWEDSAHPQRPDNADLAILYAVVDGVPPARWKEFMRFMGLSEHEIERLEMQNGRCLREAQYSMLEAWRRRTPRHEDTLEVVGLVLSKMNLAGCLENILEALRNPAPSSTTRLPR

SEQ ID NO: 12 - DNA, 42218 bp, Homo sapiens
  source 1..42218: mol_type = genomic DNA
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtgggtgactcgcgcggcccacgggggacagccgccccgcatgtccacccggctggtgcgcagccttcgggtgcccgggccgcgctctcccggggcgctgtcacgggctgggaggctgggagtcccagtcgccgccccccatccgcatcagacacgcgcgcctctggggacccgctggggactccgggcccggcacacgtgcgctcggggcacaactctggcccccgaagcccctcttacccgcgtcgtgacaggggcgcaccctggttcctccgcgagcgcaggccggggcatttggggctgagcagcgggtcctgggcagacccccgctagacccgggacccctcttccctcaccccggcgactgccgcgacagcccttccctcccacgcggtggagaaggggctgtgccggggcgctgccccactgcccacgccgggcaccctcttcaaaagacttccttttcctctgggcgacggttttccgatttcttagaaataccagggtcctgtgtctgaagagcaggaccaggtgtgtgtcaggtccactgagggcacagctggagggcgagctgcctgtctgcttcctccgatgcgtccgggggctactgcagagtctgttttctgcggcctctggggacagggcaagcgagggaggtctgggctggtaatcgagcaaggcaaatgaagtctccgggggccgcaggaaaatggagacagcagaaaccctagggctcctattcaatatgaacgtcactcccacagtagcaacgggcgtacaagggactgtgcaccctttgcctgtttttgactgtctcctggtctggtaacataggataaccccatttttgttttactgtctccatggtttggtaagataagataaaccattttttagagaaggaaaactgaggctcagagaacttgtatttcatggaagggtctggggggaactcttcctccaaagggggcctctcctgccttcggcccctgtgccctgaggctgcggggaaggtgggcgttcatccttcctcagagccgcccctgatggacacatggcccatctgcccccatctgacatcttgaccgaggaggaaggtgggaggtggcactggcttctgtggtgacataaggtgacttgttgatctgaggccagagagtccagttcttgtgggtggaagatgggggctgtcagactagaattgactcaccgacagtgggtgtcaggagtgggtcctggagaactgccccagagtaagtcctgggtggcctttgggtcaggagagtccccactctagggccgagtgtgaatggggacgaccgtggtccccagcttgactttggggtcatgccatatactctggcctcagagggggtcttcctgccgctgagcttgggcctgcctgggtgaactctggggtcatgtgagcccctggatgggagaggggtgtcaggcacagggctcagcaggacaccaggctgcagtcctggagtagctggaggaggttccctcccacttccttcccttcctgaccagctcctgcggcagctcatattccctcctccctcatccctgctccccacgaccccacccggactattgccgcagccttgtagctggtctcccggccctgccccctctcccgctgatccttggcagagagacccttctcaagcatgaggcacctctctgtttgcctggggagcctcctgtggctcccaatggttgaggcagaaactcccctggagatcccaggccagagtaaggtacctgaactcttcctcgcgcctccctgcacatgtgctcccccgcctctgtagaactgatgatggtcatcgtgtgtgtcattgtgcaaaaagcattgtccgtgccatcccctctccaagggacacttcttacccctatccttcaaggccttctgcaaatgccattctctccaggaaagcctttccgcattctcagttggatttgatcttctcttcgaactcccaggcccttttgttccctttgaggacagattgcattctgctttttaatctagctgtgttttatccccctcagtggatggaaggtgtcttcagggccagaactgtgggttcctcttccttctcatcagtagcatcgccatagctcctgatttctgaacacccactaaacaccaggcactgggctaggtactttgtcacaaaatttcactgaaacctcccttacagcaactcccaagaggtgggtggtgccgtttgtctttagagatgtggaaactgaggcgtttcccaggatcacagagctgggggccggggagctgggggtgggatttgaatgcaggtctgcctgattcccaagctcctttcactgcaccctccccgtgcccgtatctgcactcatgctcccagccaggccctcacacacagtgggttctcaggatggaagtcaactgagctgcagaagaagcccccaggagacctgagctagccatggacgcagccgactcactgccagcagggcttttgccccgtttaaaagtcttatttgccagctggtatggaaaacatcattcattcatttagccatttattcatttgacaaagaaatgccagtaaacctggcagtaggtgagtgcttggtgccctctgatggggatgagcagggtctgggctgtctggggaggaggatgaggggtctctgcctgatgggcctggcccacctacccctggaagtcttggtgctgaggtgccactatttgcctcctcatcacccagggcctggggccagtttgggttaggggagaggttgggggctcccaggctggatgtcaaagaggagttgggtgatctggggtacctgctggccctgggggctcttccctccttccaccacgtctctgtctctttgccctctgtctgaaatcccagtggggaccctgtgtgttgactgagggtgtggtctgttggtcaggcacgtcttcagagggcaatgggtaggtggccaggcaccctgaggcagggccattcattcattcattcattccccaaagttcatggacacacctagtccaagcccagcccagtgcctggctctggggacctcaagatgactccagctccaatcctctaggagctacactaccaataggtcacagacctcacagacctgggctctgtcccaactgcccttatgagcttagctgagctagtaagcctatgttctcatctgtggttgtggaaagtattccctgcctggcctttgtcacagagccatgggtgtgaaaggctgagaacctgccgggcattcttacgtcagaggtgaccctgccatcctggcacctggcacagtggctccccgtagcactgtccctccccacggctgggtgacgctttatggtccttactatgcaatgttttctggaggagtagcagggacggttgtgcgtgggaaggggaagagttttgccggagccatggcatgtatgttacacccgactctacaccaactcacttcttactttttttctgagatggagtcttgctctgttgctcaggctggagtgcagtggcgacatctctgctcactgcagtctccatctgcctcttgggttcaaacgattctcctgcctcagcctccagagtagctgggactacaggcatgcaccaccatgcccaactaattttttttttttttgtatttttagtagagtcggggtttcaccatattgcccaggatggtcttggacccctgacctcaagtgatcctcccgcctcagcctcccaaagtgctgggattacaagcatgtgccactgtgcctggcccgttctcacttcttcttctttttttctatttaatttttttttattgagatagagtcttactctgtcacccaggctggagtgcagtggcccaatctctgctcactgcaacctccacctcctgggttcaagcaattttcccacctcagcctcccaagtagctgggattacaggtgtgtgccaccatgcccggctaatttttcctttttttttttttttttagtagagaccgggttttgccatgttggccaggctggtctcgaactcctgaactcaagtgatccacctgcctcggcctcccgaagtgctgggattataggcgtgagccaccccacccagccccattctcacttttaagccatgcttttctgtcctcagatgtgctgcacacacacctgctccagggcctttgcacttgctttccccatcacctccaatactcctcctgctgacgtctcttggtctccttccctcgcttccttctgctgaagtagacaatcctgcctgtaggtgctccctgccaggcactctcactccctgacctgctttgtattcctgtataatgctcttgacctattctatttatttcacttttattatgtcttcccacaagattgtaagtaccgtgaggacagggactcattcaccactgtagactctagcctagaagagggtttttcagtcagtgcctctgactttaattctttgttgtggggctgtcccgtgagctgtaggatgtttagcgacatccctggcctctacttattggatggcagtagcgccaccctccctcgtgacaaaaatatctccagatattgctaagtgtcctttggggttgggggattgcccctgattgagaaccactggcctaggacagtgcctggcacatagtaagtgttcaaaaaatattcatctgaccttgaccttgataatggccctgttttacagatgagacgggggtttgaaaagtcagtggtggcagggtggggttctgtttgcctccatctcttggggtatttttctgggctcctcttcccattgagtggtgtctgctgaggttaggggtcctctccccaaattgctcaccccctcgacatccctcgtgtagtgagggatggggccactcactgacggtgactcagggccccagaatgcatgtgatttacccagaagcgggcaaggagggaagctggggtttggggtgccggggatgaacaggaacaaaggagaggcgctgtggctccccgttctggaccctcgtctggccacagggaagcagcactgatgactctggggaaaggccccggcctctgaatcagataaacctcaagtcgctgctctgctgttttactggctgtgtgacctgggcaagtctttctatccctcggagcctctgtgttcttatctgtaaaatgggagtaatcctagcctcacgggctggctgtcaggattaaaagtgctttctaatcctggcattggcagctatttgagccattggtggctttgcaggtctgacccgactctctgggccatggggtggtgctcgggcctaggtggggacccctgtggagtcagactgtctgctctgagccccagggccctgaagcttgggtggcagccctgcagttgcccagagttcctgtctaagtggctttgttgcccatcatctcagaaagcaacttctttcctccctgccttttcctgtctgtgcttctgccccagtgccccctggctgggctctcaaacccactgcttgacagcctggtcttaggacactgtattgtggcaaaggctgtggggtcaggatatcccctcccctccctttgccaccccttttagtgagtaacaggtgctgtgtgttttggggatgagtgaagggtacatttggtgaagcacagagagagtgcacaaggcttcagtttgcaagatgggcacgggaagattccaggccatctccaaaacctcttccatctccaacctctgtgctgctgaaggttctgtttacttgtgaccctgagaaggggatgccctatgggggctcttctgagatcatgctgtccattttcctgcctccaagtgacttctaagacagcatggctctgaggaaggagcactgacttgggtgttgggagatcacagttcccatctgagcaccgtcactggcttgctgtgctaccttgcttgggttacttaacctctctgggcctaagcatccctatctatgaaacctaacaaccctggttccaatgctttcctagaatattggggaagtgtcggctttgagtatacttgacaaaggccacagattgggggtggggtatataaaagtctacaggaaggagagcccaggactccccactgtcaatatcttggttccccagcttctcttcttcttgctgacctcaatctcacttgctttgggtggattgatttcctgactcaggctccagggccatcaagaattcacggctgcctttcagccttcatgaaggacatggtggagctccttaggcagaactggggtttgtggatgtcctgcctctctgccccttggtacctaggtggcaccttttcaagttcgggagctagatcctttccccttccccacactgtaggaaagtccttactgtaggcaggcgctgcagagcctgctgtctgggaacccacaggttcaaggaagccttgagcaggaagggcgttttcctggaagaagccctttgtcgctggtaatgggtgcaggctgctctgacccggttctgagtcctgcccccttccaggcctagggctttgggccttgatcacctctgctgagtagctgactgcggggctggggctctgatgctcaggacccacctctctgggacccacagtctttttccactgtggcgtgtagtgatgtcacaggtggcagtgatgtcactgtggtttgaggtacttggctgtgagccccggaggaggaagtgtctgttcgctgatggggggttggaagagatcattgacttctgccccaagcgtgagccccaagtgtgcaggggggagtgcggggggagggctgttggcggcgcatcccagggctctggctctgcccttgcatctagcctgtctttcctgtgggctgtgacaagccactctgcatctctgagactccatttcttcttcttcttcttcttcttcttcttcttcttcttcttctccttctccttctccttctccttctccttctccttctccttctccttctccttctccttctccttctccttctccttctccttctccttctccttcttcttctgatggagtctgactccgttgcccaggctggagtgcagtggcgtgatcttggctcactgcaacctctgcctcccaggttcaagctattctccagcctcaaccttccaagtaactgggattacaggcatgcaccaccacacccggctagtttttgtatttttagtagagatggggtttcaccatgttggccaggctggtctcgaactcctgacctcaagtgatctgcccgccttggcctcccaaagtgctggaattacaggcgtgagccactgcgcccagcttccatttcttctttgaaaagtaggaggggttggaattgtcaccctggaggttcatggttgctagattattgattctgttgctggattcaggagacctgttagctggctagttcacagcaagtattgggtgtttggaggggggctgcagagccccttctcaggccccaggagggccagctgccctccctaccccctcttttggacactagtgggcatgttctgctgggaaaacagacagtgtaacctgacttcgagggctgcaggctgaatctctttcagatgtactggctccttgagaggcctagcagatctctactctgtgggtccctttcggagctggggctcaggtttgacccagccactctgaccggagacagcgcagaaatcaccaggagcatttgtttgttttcctttgctgtccagacagtggcccaccgtctgcttcagtctgagcgtggccctgcactagtgagactgatgtggccacaggttcagagaatcagtgcgcctgagtgggagagcagagcgggaggggatttggaggcaggcgggctgtggacagggaggggggaggacatcttccgagctgtcagcgggaggccttcttgaaggtctttcaggagcaaggccagaggccgtgggagtgggcggggcctggggctgggagtcaggggtctgaggcctcatcttggcccggcctctgcttccgggagaacttggccaattcccttctcctttctgaaccttggtggccccagctgtcacttgagattagcaaggacctccctggtcccagcaggagtgagtggctttgggcagggggtggagtgcagattgcactgccacgtcacccagctcagaaggcccaggtgtggtccagctgtggacttgattgtgaactcctggggaagggctctggtcctttgcctcagttagcgggacccccaccagtgccctgggtgcccctgctggcccctcctctcgctgaccccagccccagtttctggctgcaactcacagaggccccaggctcctgtggccccatcaccagcagctcctgctgtcatttcctgagcctcgcaagcccctaatcctgctccttcctgtcctggcccccagctccctcaagccccactcctgcatcccattctccctggacatcacccctcccaccccttggctcaatttccttcctgtcgtggcttggtcctttggtcattgccctggtcttttcttgtccccttcctgattctgcccttctccaggcccacacggggaacctcctaaccgctgagccctgaaaggtaggcgggtttaggatgagtggctacagggaggagaatggtgtggggagagagatatggaggaaagaatattaggggacaggccgggcacggtggctcacgcctgtaatcccagcactttgggaggccaaggcgggcggatcacttgaggtcaggagttggagaccaccctggccaacatggtgaaaccctgtctctactgaaaaaaaaaaaaaaaattagccaggcgtggtggtgtgcacctgtagtcccagctacttgggaggctgaggtatgagaatcacttgaaccccagaggcggaggttgccgtgagctgagatcgcaccattgcactccagtctgggtgacagaacgagaccctgtctcaaaaaagcaaaacaaaacaaaacaaaaaccgaatattagaggacaatgttaggactgtcccagctgtcctagctggagccaaaggtttgttggggacagtgacagcagcacacgaggctgtaagtttagggccagtttatggaggagtagtaccaattacattgagaaaactgtagcaactataatttgggaggaattttctaaatactttacatgtgttatttaatcttcccatcagccccaggaggtatgtgcatttatcagtcccattctacagatgaaaagactgaggtctatgaactcctttatttaaaaacaaaacaaaaccccctcaaggctgcctgctaagaggggaggagtgtggattcaagccagcatccttattccagtgtccatgctctcagccaccacacacacactcctatcaggagttggggcagtggggagccattatagtttttgagcaagagagtgacagggcaaagatcactgtggcctcagtgtgaagcacagatgagatggagagactggaggcagggaggcccatgaggaggctgtcaacatggttcaggtgacagctctgggttggtgaagaagcagagaacaggaaggaaggggcgagaggcttgagggaggaggaggaagcctttggactgggccgtggatatagggtgggaacataggaggtgaggagcatgctctctccacctgctttttgcaacatgcggcctctctggccttagctctgcctgtcctgagccctgagccctgagcccaatggaccgattctgttttttttttgagatggagtcttgctcttgttgcctaggctggagtgcagtggtgtgatcttggctcactgcaacctctgccttctgagttcaagcgattctcccacctcagcctcccaagtagctgggactacaggcgtgccccaccacgcctggctaattttttgtattttttttagtagagatggtgtttcgccatgttggcccggctggtcttgaactcctgacctcaggtgatccacctgccttggcctcccagagtgctgggattacaggcgggagccaccgtgcccggccccagtggactgattctggtcagtgtcaaccccatttgtccctggagcttttgcagtcggtcacagggccagcccggtcctggtcaccgctggctggttaccccggctgggttgggcaggagtctgggcagggctggttgctcatctcaggtatgaaattggctgcacccccactagtgtgggggagcaggattccagggcctgcctggcgctgctacccctgtgcctccagacccctcgagctcctcccagacctttggcttcgttgactgtgaggtgaaaacaaaagggctgctctctgtcacttcccctaggggcgggcagatgctggcagggcacagagagccgagggctaagagctgacatttagcctggctactcaggagactgaggcaggagaatcgcttgaacctgggaggcggagtttgcagtgaaccgagatcgcgccattgcactccagcctgggtgacagggtgagattctgtctccaaaaaaaaaaaaaaaagaagagctgacatttagcgggtgcttcttgggtgcaatgcgcaagagaccgatgcattaactacacagagtgcccttccttgtcacagctacctgggggcaggggaacatcatccccattttacagaagaggcctttgaagcacagagattaactgacgtacttagggttttgcagctcactagaggcagagccaagactccaccccaggtggccaggctcctgagcgctatgcctacctcaccctccaatgaggtggtggtggaggagaccgtgagccgctgtcacctgaacactggtgcactcagtctgctgctctgaggacgccgccccctctctttgtaagtccatttcctgcccatttctcatgctagctccagctcatttcaactaattgaaaattaaaactttgcttctgttgagcaattcggcatcgatgagacgccagctgtgggctgtgtgctggctgtgtgtagggtgatgagtagcagaagcacgggtgacactgagctctttccactgccgggcacggtgccaagcacgccacgtatactgacttcatgaatcataaagccctgtgaagggggcactgtttctgtcgccattttatagatgataaagtggaggtgggggaatggcagaactgggatcaaagcccacgtgtctgtgtctggctcagcgcccatacaagggtgtcaaggttgcggggaggcacgagggaaccaataattaaccaaaactggtcgggtgtgggcagtatagagatgcacccgggttgtcacaggagcccctaggagggacctctgacctccctgaaatgatggcagagggaactcttgaaagaagctgagaagtcagcactgccaggtcgggggtggcgggtcaggacgtttgggcagaggcagcagcagtacgggcatggagaagggggcggaggagccaggtgctgctttgtgttgtgacagcaacaggtgagtgatgaatacaggtgggagatgctggcaggggctaaggaggttgcgcaggtggtggcagccgctggaagcttttaaggagagtcaggcttgattgtaggaagggggcttaggtccatctgaaagggacagaaggctgggaaccaagaccaaggagcctgcagctggcaggagatgctgagggcagcgaggaggaggggtcgtgggagatggagcaggcgacatcagcaggccccttggagggtgacggtgggagaggctggggcgtctctcattagactcagggttccttgcaggcagggaccatgcccggcgtctttgtaccctggcatttagcacaaagcctggctccaagaaccggataccagcccagtcttcctgtctgcccccagctcatttctaaagcaacatcacaggcagcgagtcgggagtgggatgtgtcacccccgggttgcctgcctgtgatgtggtcagtccttcctaacgcagatgccattcctctccgtgacggcagacgggcctccgctgtcactggccctggcgttgctgtgacgacctggcctgatgtagctgcaccctgcagtccatggggctcctgtgaacccccaaggttgagacccaaatgcctctgctgcctcccaccacgccctaggggccccgcgacccgccatgcaaatgacgtggtttccgcattgcttgaagagggtttcctctgtgtgggggtccctgggtccttgggcagatacctctgcccctccccacagctttggcccctgccctaagaccctggagacccttttgcctgggagagtcactgaaccacctgcccctgctcacagtgtgttcacagaagagtcactcccctctgtgggctccttgtctgtaaaatgagggagcaggacaagatcacagggtacaaaactgtcagagggcacagaggctgatgttgctgccatcagctcctggagtgccgctcactggctgcatgaccttggacaatttgcacgaaatctcccaagcctcaatgtctgcatctgtaaaatggatcatcctgacctcattgcgttagtgctaggataaaataaagagaaggtatatgaacgtgcccagcactgtgcctgcacacagtaggtacttttcctggatctgaacagtctctgttcttccagctctgtgtaaaggcttgatctcgacccctaaagcctgaagcttctccctgtggccacctccccaccgcatcatcttccgttctcagcgggtagatccctgcccaggggggaccccctggggaaggctgggagcatgacctcttcccaagggtacggcatctaaaagggatctccggaacattgaaaagccatgaaggccgggcacagcggctcacgcctgtaatcccagcactttgggaggctgagggaggaggatcgcttaaggccaggagttcaagaccagtctggacaacatagcaagacccggtctctttctataaatgaaaaaataataataaagccatgaagatccccaaagaaggctgtgctaatggcggccggaagagcagagggcaccacagatgccgtgctttcagctttttccttctcctccaggcctcccttctttccttcctctctcgctatttataaataagagaaggcattgagggcccccacctcgggtacttgcagggccttggcctgggaacaggacaagcagcaagtcagttgacccggggttgctcaattccccaagagctgacgcaaactccagaatcagacctgccctggccacctggccctgccccttaccagctaagggctccagcagagtcctagtcttgagcgaatcttatacctgcagccctgtccaccagctgtgcttcctgagtcctccagggtgagggggcgtggggtcagaagagaggggacggtgtgcagggagggtggaacctgactgaccgggtcagccttactcttccagcggggtcttttcttcctggagctcggccctcggcagctctcagaagctcaccttcttgcaggctctcacagccgcatcgtcctctggctgggctgctcacagaccccggcccctgggttttgtccactcacccagactctgttttggggtcacattacccttccagtgttcctctgagcaaagtccctgtgagttagcccaaggtccctgtcaaggaggaccacatctggcctgttgcactcttctcctttgccccaccgaagcctctgtcacactgtcacagcagcgggcctgccatggtctccccatgtaaggcttcctgaggaacgggcctggtgccagcctgggggtgaactgccagccccaggagtcacaggtcagcttccccacacttaccctggaggcaggatggaggccccctcctttgcaaggcccacacgggccatctaacaccccactaaagccgggcagttcgctgccctggggtgtgttctcgccttgccagttcttggctaaaaccaaggcaggaagagcccagttcccatgttctgtaacatgggtctatgaccagggcctttgcaggccccttgcagcgcccccatggagcccccaggagtctgggagaagatgatgaacccaagtgaggacctccccaggggcctttgctcctcacaagaacccctggaggagacagggcaaggagtaccactcccattttacaggtgacaatgccgaggctcagagaaagtccatgacacccccaaatcccccggacccactaccctgacctcctggcggtgcacagagcatgccacgcacctctccatatgcacatagaccctctaacctaagtcagcgccccttctacaaggccggtgtccagcacacctgggaaaggggacatttctggccctttcctctcagaagtcgcctgctcgagctgccctccctggggggaaggggatgggggcagaagctgagcatggaagcccctgctaatccaccctctgctctctccagaggcccaggaccagcccaactctgccccacatcccagcccccacctcccagctcgtgctgtttctccacctctcatgtggatgggccacacaccacccagcacccctgaggtctggtgagggactgagccttcgacacccatccttaagaacccagggctggtccttttgcttagagcatcaaccaggcccacactctcattttacaggcagagaaactgaggcacagagaacctggttactgcccacagcagaggagacacttgagtgaggacacagaggatctttggggtaggggcgggccttggggttgagacccagtcttcctggccagggaggaaacacaggactggaggtgaggaggcctctttgaggctagtttcttcatctgtaacttgggaataacaataggactggcctcagggggaccaggggaggattcagagaggctatgatgctgggacagctcttcgtgatgactggcatgaagcaggcttctagtagatgctctactgctgctttcgttctcaccctcacccacccgctctgggcccactgccctgacctcctgaccaagcactgagtgtgccaagggcatctccctgtctccccggccccgcaccttgtgtcacccttttccttagctcttaccaccaccttgaattctctattgaacgtgcatgtgtctccccagctaagatgacaacttcccgggggcagggatttgatttgttcaatgctactgtttacccagcacctacaacactgggtgctcagcacgtctgttgaatgaatgaatgaatgaatcactgctttcacctttgcccctgactttctccatggaccacggacaagtgacttgatctccctgagctgtgaaatggggccattcacaccagctttttagggtggaactaggattttatacagtgcctgccatctggcctggcacatagtagttgcttagcaaatatcccattgttttccttcctccccagagggccaaagccaagacctggaggagcaacagggtgggggtctaacccctgggtgacagagacagctcccatctgggatggaggctgcatggtgggaggagctgctgaggttgggccattcagctttggggcccttggagtcctgggtctgtgtggagggggaagggaagctctctgggccccaagagaagggaggcaggattgggggtggcaaggattgcagctcttctgggatagccttgggcacactgctttgtctccctgagcacctgcgaatgtcagtgagaagaccagacagtgggtctaacacgcacagttgcccctgggaggtagcaggtgctcggtaccgatgagctaccgctaactgcaggtgctacctgtcttagaggagggagcccaatctggaggaggggaaatgggagctgtggacccagatgggaggcggctcagagactgtgggtgggccctgatgagcttcttatccatagtcctctatccgcttgccgccatcctggccctggcatggcccaaagtctctgggagacgaggggaggtggctttgcctttttccatctctgttatctcatctgtgaaatgggcacaatcagacctgcctcaccaattggaatatagatttaggtgcagttttcttttttctttccttttatctattctctctctctctctctctctctctctatgtatatatgtatagctatctattgatattttctatcatccatctatttatcttgtatctactttctattatctctatcatctatttatctaatctatcatctatctagctatctttctattttatctatctatctatctgtttgtctgtctgttttatctatctagctattttatctattttatctatctatctatctatctatctatctatctatctatctatctatctatctattctatctacctatctatctatctatctatctatctatctagctagctagctagctagctatctattctatctacctacctatctatctatctagctatctatctattctatctacctatctatctatctatctatctatctacctacctacctacctacatatctatccatcctctccgattccctcaccactgggtctcagattccctgcctgttaaataaatggtcagttaaccctcaacagtgattttcaatttttcacttgaggcccccatattttggaagattgtagtttgctgaacacgctgactgaatcaagtactatccaacttatttcctcattttaacatgtcaaagatcccctgtggggtgagtggggtgtgaattggagcctccccaatccaaggcacaggaactggaagcttctgtagccctgcatttcctagagaatgcttgccatgttttggggacctcctggggggcccctgtactgaccagaggccggctcccagggctgtggtttgggcccagccccctgctcctggggactgctgctgtggagggagtgaccttttgttctgctttgccaaggggctttctgttcctcattgtttagccatgacaaggctgtggatttttttttttttttctgtccaagagaataaacaggcctgagattgctcaagggagccgaccccctctgccagctactgggggctggaggctcacagagccagggaggaagtcctaccccctctgccggctaccgggggggctggagccacacagagccagggaggaagtccagggacggtcactcacagcaaagcctcagacaagagaggaacctggaggggaggaggtgggaggccaccactcagccggcctgggggccatgggagtccagctgcctgccgttagtggccaggagccctcgtccattgcggctcttgagagcccaaacttggccacggcagggctctttcccatcaggccctttgagccctgatgggaaacccttccccagccagggtgggatttcttgctgccgcctggagccgccctggccctgtggttctcaggccctggaggagtgaggggctcccctggttaccccaagggcactcgtggggatgaagcccgcgggatgcaggattcccacgagacctgtgctggccactgtgcggggtggggggctgccagggaagaggtgcagcccctgcctgcctcagggatgcgatctgagggaagaggcagctgtgtctgggaggccgttcggtcggagggggataggcacagcccctgcccttggggaagcgcccagcagcagccttcagggaaggcagtttgggggtttgccgagggccaggaagaccctctgccgcagttagtattcgatggaatggcttgtgcagagggagctgcagaagctgccatttctctgggttaggggctcccacagcctttggatgccgggtctcggccatgagccccctccgccacccctcccccaggtcaccttgctcaatccaggaggccggatgatcagagtgtgttctgcaaaggagcgtgagaatcactggggagatttcctttttctttttctttctctttttttaatggtacagcttcctgggcccacccctgagattctgcacttgtttggggagtgggtgttggcttcatgacaggcacccctgggtgctttggacacaggcagtgagctccccccacactgtgtgaacccctgagaaggcttctccttttgtctagggtgcctgggagtctgctccagcagcaaaccggggaggggaggggtactgggaccgagtgtgtgtggcagcaccgtgtccaggcaaactgaggctccagacgggggttacttcttcagaccctgttatgcagcatggttcccactgcccgtgcccagaattcaccatagctgatgggcctggggtgggcctccggggcaggccagggtggaaagtgcatctgtgtttgagctttgcggccctggcgctggagttagatccctacaggaaaggacattgtaacccagcccgtggttgagactagcagctgctgagcacctacgccctgctaggctgtggggcaacagcaaagggtaacccccagcctgcagcctcaggggggcttcccatctagcgaggggcccagggaggccatgaggagagccatgagggaggaaggtggggactctgggcctgagtgactccttcagggtggggtgcgggcagggagccttcaggagggagggtactgcctgagcagggctcttaagggacacgtggaatttgttacaggaagcaccaggccattccaggcatagaaatcagctggggtgcaaaggcctgggggtcagaagagtggcagattgctccatggcaggaagccccagctcctctgggtcgttcccgctgagggattccagctgttggcaccgaggggtgcagccagggcagggtggtgctgcctgccaggctgaggtgggagacagctgggtttctagccctgcttgtcactcgctataggtgcagccaaacgctcagcaggcccaaactctgggcagtcctgactcccaggcagtgctgtgaggaaggggaggaggaggcacactgaacccacctgctacgttggctggtggaaatttggccaggtctgcactggcccagcggtgctgggtgtcgtgactggtgtctgctctgactggcttgctgacagctgaagtccaggataggtccatggccacgttgtgctggtttgttaaatatttttagtatctcccctgctcctatatgttaggtactggagtccagagaggttaaggcacttgcctaaggccacacagccgctaagtggtggagtgaggatttgaaaccagttctttctgactccctgtatccttatctatggagaggccccatgactcgttcactaaaggttatggatctcgatcttgtactggccacatgactgcagctggcagtacagtggggagtgcgccagccctggctctgacctcctagaactgccctttgttcatttccaggtgacgcatctgtgggggcccctgaaggacttggggtgtgtgtgtacaccctctttgcttgtacagatgtgttcacacccatgtttgcctacatgtgaatgtctccattctgagcttgctctcagcctggtagaggtggctaattaatgcttgtccctgaaagcccggactgccaaccccagtctttcaggagagagggcaggactcaggggcttgctcccgggggtcctgggaaggcacaatggtgacagtgctgcagctctgcactcctggagggtcactcagagacccgagagaggagggctctgcgtctgctcctctgtccagggctgtagcttctctgggtgcctttgcttttctcttttctcccctctttttttttttttttcgagatggagttttgctctgtcaattaggctggaagtgcagtggcatgatctcagctcactgcaacctcctcctcccaggttcaggggattctcctgcctcagcctccagagtagctgggattacaggcacacgccactatgccgggataattttgtatttttagtagagacagggttttgccttgttggccaggctggtctcgaactcttgacctcaagtgatccacctgcttcggcctcccaaagtgctgggattacaggtgtgagccaccacacctggccctctctctcttttcattcatcacattgttcatctcttcccggaggcatccagcacagtctttgagactgtgagctgtgaagtcagacaggacccaggtccaaacccttctctgtcacttaccaggtctgggaccttgggcaggtgacctgccctctcaggcctcggtttcctgttctataaaatggctgcagtagggaaaccgtctccatcttcattgggtggttgggaagtcttgctgagaccacacacctgtctcaggtagggtcttccagaagcagagcctgaggcagggatctttgtgaaagtgatttgctagggagggctcccaggagaagcaggcagggcaggtcaggggagggggcttagcagggacgttgttcaccagcttcagcccaacaccacgggggtggctctggaccatgaattgtaccagggtgggtcccaccttgaggctgtggagcattcattatcagccaacacccacagcagctgggggagcggtgccctgctggcagcaggggtctgagtggggtgcaacagcacccaccaattcgagcagcagaaacgcttagaacagtgcctggtacctaggcagtgcttcaaaaatggcaacgattgtcatggtcataaccaacattcagctgatgagtgccggctctacggtgggtcccatgtgagagattgagggtctagagatgacaaggctgccacctctgctgtctgcatcacatgagtgtgtgtgtacacatgtgtgtgcatacatacccacagggggtggaaggacaagcaagtaaccattccagaacaatgagatgactgttagaggggtcataacccacgtttagagagagccgagaggaggaggaggaggagcttgaattagccttgggtagtcagggagggcttcccagaggtggcagcactcatcttgaaagacaaagggatgtttcaggtgcaggagggaaggagaggtatcttccttcctcagcagagggcagctttgtgctgagaacccccgttctctgtgaccaaggccactgtcttctggatatctgtcccatgcagtgttaggggtcacccagcactggagaaggccagcagagtcagtgcttctgcctgcagacatgactagggtacactgaggtggggaggcaggggaggtaaaagaaggcacgagctctccttcctgtaccctgctccagggggagaaacctccccagccatcatcagtgcagactggcagggggagggccaaacatttgcagggcggggacctgggcatcaggcatggcagaacccaggggcggccctgttgatggcagtcttcccttcttccttccaggtggcatttacaccctacgccccggagcccgggagcacatgccggctcagagaatactatgaccagacagctcagatgtgctgcagcaaatgctcgccgggtgagggcagccacgggggcactcggggcccatgccctggaggagcgtgtgtgtacaggggctggggcgcaagagcatagcccttgattcttactgaactcctagttctatgcactggcccatgctccctgctgcttctgggcctgtgaacatgctgttccctcttcctggcacacttcccaactccctttccctgattaactccaacttgccctttagacctcagctgaagatgtcccctcttccaggaaggctgcctgccccatactgcccagccggtggaggagggggaggaggagcctaaattagctcctgggggagtcagggagggctgcctggaggaggcagcactcagcttgaaagacaagtgctgcctcctttgagctctcactggcctctgatgttcccatggaagctctttccttgctgtgggattatagcccggtcctgcttcctgttgcctccactgtgtgctcctctagggcagagattgtggcttgttttctgctgaatccccagcacgtggcgcaatgccatgtaggtgctcagtgcatatttgactgactctgtgaatgcttacccatttcacagatggtagacctgaggcccagccacagttggtggagctaggtagcctgactcctggtgccctccatctattcatttgtgtctgtattcattcatgtcatgattataaggtatctgctgtgtgttctaagtgctgggaatacacagagagcagggcagacaaaacccttgccctcgtggagtctgcattttaacggtggatgcagacagatacattaggagatgtgtggtcctgtctatctgtaagtgctgtagaaagacaaggtcaggtaaggcgggggagacagaacgggacatactgttttacacagggtggtctgggaaggtctctctgaggaggtgacatttgggcagagccctgcaggaggtgagggagcaggtcatgcatgtatacagggaacagtgtgccaggaggaagcaacagcatgtgcgaaggccttgaagtcggagcctgtttggtgagttggaggaacagcagggccagcatgtgtggcccaggatgagcgaaggcgagtacggtaggggaggaggaggaaggagtgagcagggaccatggtaaggagtttagattttattctgtctgggagccaatggagggctttgagccgttaccttgagatgtaggtgggcatatgagtacctgtcaccgtccctagccattttaagacatcctagggaaagtgactctcttctttcttttattaactggaagggtctctttaagaagttactagagaggctgaggtgggcggaccaccggaggccaggagtttgagaccagcctggccaacatggtggaacccgtctctactaagaatacaaaaaaatagctgggcataatggcaggcacctgtaatcccagctacttgggaggctgaggcaggagaattgcttgagcctgagaggcagaggttgcagtgagccgagattgtgccactgcactctagcctgggccacagagtgagattctgtctcaaaaaaaaaaaaaaaaaaaaaagcagctactgagtcaacttttttttaaggtttccgttggtgttctctgtccctggaatgcctgctttccaggcccatgcccttgtccttgataggataccagggactacattcttactacctgttccccctatcattcatgcccctagctggagaatattagccatctatcaagaaggaagaaactggcaagctagttcttactaaagtgtgattgactgataatagggctccagtggtggaggttcaagcatctggaaagagcagagaggaatctatttttgagcagaaggggttcagaaatgacgtgatagctggtacaatactagatgggagatgattctgaggaattggaactccacagaatagccttcccagctgggctttagaactctggactttgtggggacagtggatgagcccagggtcctggcagaaggctcgcccagctgagacctctggcccttgtttcctcaggccaacatgcaaaagtcttctgtaccaagacctcggacaccgtgtgtgactcctgtgaggacagcacatacacccagctctggaactgggttcccgagtgcttgagctgtggctcccgctgtagctctggtgagtaggttcagagaaaaagggggcccttacacccctgcctccaacttcccccggcaactccagcctctttggcttccagctgtctggagttaccccaggctggttgttggaagtggcacaggtgcagctgtttacccctaccactggcattttcctcctctgtctcaccaaagcctcttcacagccccacggggcaggcggtgggagaactgtgcccacgtgagggttgaggaggtggtgcgtgggagagtggtgcgcatgctcgtgctgcgaggagcaggactgcggggaggagcgaaactgctgggaagagcgggactgcggggaagagcgggacttcggggaggagcggcactgcggggaggagcaggactgcagggaggagcgggactgtggggaggagcgggacttcggggaggagcgggactgcgggggacgagcgcgactgcgggggacgagcaggactgcggagagtagcgggactgcggagagtagcgggactgcggagagtagcaggactgccggtcctgcccctggactctggccggtgttgtgtgtgccccatgctgaggcggtccgccagcctcctggagatccgctgtctgagagtgctgggctgtctgggagggcagcgtgggtgtggcggaggcaggcgtgaccgtttgccgccctctcgctgctctagaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggtacggggtggggctcaggtccttggggacgcccatgggcctctcctttgtagacatccttgcagtgtcacgggcatcaacccattaattagtccagcagggagcactgttagtggtggccaggtgcctgctacccatccgtctgtccacctgtccaccgttcattcttcctgccagcactcccgattaggcacctcttatgtactggaaccaggagacccagagatggatcagaccaagccctaagctaggaaagttatgtgatgctgcaagatgaactcacataactctacagcccaatcccgtgcatgtgtgtacaggaatctgtgtgtgtgcatgtgtgtacaggcatctgtgtgtgtgtgtgtgtgtgtgtgtgtgtgtaaggggtggaggtgcagacagagctccttgggcccctcagacctctcctagggctctagtgccaaggcccagctgtcccgcagagtgtctgagtggttgacaagttcggattgttccctgaaggaactgaaacatcagacgtggtgtgcaagccctgtgccccggggacgttctccaacacgacttcatccacggatatttgcaggccccaccagatgtgagtagctgagtcctttggttctggaggagcagggaggggctgtccctgggtgactgtgggtccaggacacagagcagctcaccaaccaccattgtccagactgctttatctgagggtggctcccaggataaatggcatggtgggcaggaccttgccctggaaggcaggactgggtgggtgcatggggaggtgggggagggctaggagggggtggtcctcagagagggcacacatcgtcactctcctatcctgcctgctggggcccgtgaatgagcccagccaccccagccactctgtcccctgctgcctcctgaccaagcctcctcctcctccagctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggtaagtcgcaagaagtctcattcattcactccttctgtctgcctgtctttctgtctctctttcttcctctcccatggttttactgagagcccacggggggctggaccctgtgccctgtcctgggatacaggtggcagtgagactgctttctccccacacctggtgctatcattcaaaatccctaagtgcgtgggctggatgcagtggctcaagcctgtaatcccagtgctttgggaggccaaggaggctggaggattgcttgaggctaagagtttgaggtcagtctgggtaacacagcgagactccatctctataaaaaaattaaaaaattatttgggtgggatggtgcctattgtcccagctactcaggaggctgaggtgggaggatcacatgagcccaggaatttgaggctgcattgagctctgatcgtgccactgcactccagcctgggcaatgagtgaggtcctatctcaaaaacaaaacaaaaccaaacaaaacccctggataggggagaggtggggtcagcaattcacctgaatatgcagatggtctccttgctgctgtttctgatgagcccattaggctaagaagacagcgggctgggccaggtcacctccaggggcttccattctggggtagaactgcatcaggccagaggtctcaaactagtggcccttaggctgaatgaggcacacagacatgattgctttgtaattggatttgagaatctccggcctagactcgcccctcatttgcaatgacccgaagcaccttgggtccctggcttgcctggctggcccctggtacatttgagtttgttttctgtagctgtctgagcttctcttttctttctaggactgattgtgggtgtgacagccttgggtctactaataataggagtggtgaactgtgtcatcatgacccaggtgaaaagtaagagtccatccttccttccttcatccacttgttcaggaagcttttgtggggtgcctcctgtgagtccagcactgggcacagccttaggggtcagacagagctggatatgggggtccttgttctgtaggagcttaaggcctggggtgaaggtacctctgctaacttaattactaaaaggcaagaggtgatcagggttttctaaagggtaggaggagttcaggattcctatccaggaagcctgggggattcgggcttcctgcagcaggtggtctctgagctgggtaagatcttgatgggtggagtctgtgcagggaaggacacgcatcctcagtggaggcgcggatggtatagatgggggactggagaatggttgggatggtggcaggtggagttgggtgggggcttctgtggaccttgtctggtaggcgattggtccccacagggctgggcctctctgctggttctatggggccccatactgccctcctacttaggacagatgtgcctgaggaagtcaatctcttacttgtcccctctcctctttatagagaagcccttgtgcctgcagagagaagccaaggtggtgagtgtctccactgccctctccccctcttcccctggtctccttcccggcgtgctgggctgtcacagaggaagcagtgaattctgtcattggtctgtccagtgtcaggaggtgtgcagagccctgggaggtggaggaaaggccggggagtcaggcagccctccatgggctaagagaggctgtgagtgtgaaatgctccggcagagagcagatgccttcgtggcgttcgtggcaggcacgtggtcgtggacccacagcgcaaccttgggcagatgtgaaattgctctatggctacttccttaccatgagatgggggttgtgtctgctcgatccagcctgcagggtggtgccattcggtcagctgacaatgatgtttctccttgcctccattgtgtttacagagcactttctcagagagcagtggttttcaaactgaattctgggatagcccagtgtcccacagagggcccttgggcgtgggggaagtggtcatctctcaggtttgaggtagaggctgtggggctgggccctgggtcctcccaaagcaactcacctttgatctgttttataaagtagggcctgcgaaggctttttgttagagacagaccaacagtgtgtcatatcatagtatttcattcattctccatcaggaaggcatttggctgcaagtaaccccaaacctacaggtgactttagaaaaaatggctttatttttcttgcattgtaagaagtcaaggggttggtgtctgctggaatggcctccactatccccttgcatggtgccagcaggaccccaggctctgtcatctttagtgtgttgacttttgttctcttgtttgtggcctcatgtcataagatggtaatcatcacttcatatgtcacctctaagtttgaggtgggaagaaggggcaggtggccaggtcagccatggtgcccttatcagtaaatcggatgccttcccagaagccctgggtagacttctttcatctcactgaccagacagtgtcacatgaccacccctagctgcgagggaagctgggaaagagggtgttttgcttttccagcctctatggaggaggaggacaagggaggggggttggaagtgggtatggggtgagccagaacatacgtctgtcacagccttgcacctggtaggcagggcaggttttttacccaatttgacagatgagaaaacagagtctgatgagttttctcgacctcaggggcaggtagagaaagaacttctcaacctcatggatatttgagacaacatcttacttaagggaggccaaggtgggaggattgcttgagtctaggagttcaagaccagcctgggaaacacagtgagaccctatccctaaacaaaattaaataaaataaatatttttaaaaaggatcttggctgggcacggtggctcactcctgtaatcccagcactttggaaggccgaggtagatggatcacttgaggtcaggagttcaagactagtctggccatcatggtaaaaccccatctctactaaaaacagaaaaattagccaggcgtggtggcacgtgcctgtagtctcagctactcgggaggctgaggcaggagagtcgcttgaacccgggcagtggaggttgcaatgagctgagatcacaccactgctctccagcctgggcaatagagcaagactccatctcaaaagaaaaaataaaataaaataaataaataaaatatcttacttagttgtgggggctgtcccgtgctattcagcagcatctttggtctttatttacttacatgccagcctcattcccactccagtggtgacaatcaaaaattctccagacatcaccaagtgtcccctggagggcaaaatcacccctagtggagaaccactgctcgagggaacagctcagccctgggcagcccaatacccgaagcttacaccaggtgtttggaatcagatagattgatttcatcccctgattttggtgacaagaaaagagatgcagggaggaaaactgagttgctcaaggtcgcaccgcagtgtgtctctggactcccatctcagtgctctttgccctgccctacagtatttcagtggggaggaaattttctcattccccggcgagcgagtttcactgctccctttctccagcttcaattcagtgacactgacggttgccgcctggtggcgtgcgtagagcaggtgtctcccccaagctcttccgcagcagcccagctgtcaccttccctttccccaccaagcgtttcccaatcctctgtgcactgaagatgtcctgggtgtggcctagcagcctctgcttcccttttccattttttttttctttctttctttttttttttttgagacagctatgtcgcccaggctgcagtgcagtggcgccctcttggctcactgcagcctccgcctcccgggttcgagtggtcatcccacctcagcctcccaagtagatgggactatgggcatgcgccaccatgcctggctaatttttatatttttagtagagacggggttttgccatgttggccaggctggtctcaaacccctgacctcaagtgatctgcccacctcggtctcccaaagtactgggatgagaggcataagccaccacgcctggcccctttcctttcactgttgctctcagtgttcactgaatgtgagtgactcaagtatctgttgagtgccccagtggtgtgtctagtgcagaaaaaaacccccaaaccctgatttgtggtgtttgctaatttctgtggtataaacactcacactgtagccagtttccaactcccaagaccttcctgaatatggagttgggaggaaatctgcacaggcctgagcttatcccagcacagccctggctgagccctgccaggctctttgtgactgagtccccacgcatccctgtggggcagggagcatcatcacccccattttacagaggaggaaactgagactctgggaggttccatttttcctgtaggattagatcattaggtgagtggcatagcccaggtttgaatcaggtttatgcctgtgtcccacgctactcctcaaccagcagctactcctgataagaggccagtggagagtcctcttgagctgggctgctgggcttagagtgtggactcagcacccacctgctcctcctgccacagaaagaacgtccagggcatgggagacagtggcgttcctttgtttagaaaacaccaacatgtggccgggcgccatggctcacgcctgtaatcccagcactttgggaggccgaggggggcggatcacgaggtcaggagatcgagaccatcctggctaacacagtgaaaccccatctctactaaaaataaaaaaaattagccgggcgtggtggcgggcgcccgtagtcccagctactccggaggctgaggcagaatagcgtgaacccgggaggtggagcttgcagtgtactgagatcgcaccactgcactccagcctgggcgatagagcgagactccatctcaaaaaaaaaaaaaaaaaaaaccaatatgtgagcttacccacaatcacccccatggacacggacacactcatgcacggacccacgtctgataacaggtgcgaacatttatcgggtgttgaacggatgccaggcactgtgcccagcgttctttattctcacagccatgctgccacacgcaggcagtatgattcttgtcccccattatatggcccaagaatcatgggcccagagaagttagtaactttcccagtgtggcacagctaatgatcacggcctgggacacgaacctaggagtgtctctcctcgccaccccagcgcacacactcactacgtcactcacagatatgcacacatgcccccaacatagacacacgtgtgcacgcttcacaccctcacgcctgtagacacaggtgtgtgagccttgtgaacacacaaacagacggacacagcaggaacctggaggacctggcccgtggctctgctgggtctctgcttctgcccagcagggctgggccaggaggggccgggagctgagggtgctggctggctggctggctggctggaattctgtttttttttttttttttgagaaagagtcttgctttgtcgcccaggctggagtgcagtggcacaatcttggctcactgcaagctccgcctcccgggttcacaccattctcctgcctcagcctcccaagtagctgggactacaggcacccaccagctcgcccagctaattttttgtatttttagtagagacggggtttcaccttgttagccaggatggtctcaatctcctgacctcgtgattcggtgaccacctcggcctcccaaagtgctgggattacaggtgtgagccactgcgcccggccctgggatcctgtgttttgaatgaggctcctcagtactcggctctactggggtcccagcccaaggaataggactcagcctgcttctgtgccacctggggctgcttgaactttgcgacttgtggcttgggaggagggaggtggccgtgacctttgggggtttttgttctgcctggctgtagccaccagcagagggggtggggcacaggccagaaaaaccccttttgtggggttgtgaggagtgacaattggctgcttctcctccccttccaggctcagagcagggctggggggcagttgtgggcagtgaccagggtcagaccacctgggcggaggttcagcatgaacttgcaatgccctccatctctccaaaactgggggacccagcccagggagggtgtgggggttcctggggaagctggtctaggcttctgctcctgccacggaccagctgtgtgatgctgggcacaggatgcactttctctgggcctccgtggcctcttggggatggcttgcacgagatccctccagtcctgagtgagaggctgtggccttggggaattaagggtgcaggtggcgctcaggtgtccgagaagccatgggagccgggggctgcagggattggacagagaggaccctggtactcgcatctgttctcagaccacatctggaattgtagctccctctggagggaggcaggaggtctcagcctttcttggggggcggtggcacctgcgctgctcgctccacccctgctctcacctcccgctgcagtgctggcgagccccatcagcccttcactcatctctaccctccttctttctgcctgggacacttgttttcatcctgggcaggccaggggccagggcagctgttgggaatgtggcctgtgccatctccttttttgctgggatcagaaaacaatcgcttagaattccaaggcaagggtgtgagcgcctggccagccagtgggaacagacaacagcctgggagaggaatttccagcctctcttcagtgtgcgtgtctggaaatggggaccttgccttgagcctccagagttgaaaccccagacacccaggaaaggccctttgggatttagcccagccacagtatgtcctaaccgtgaccttgggcaagtaactcaatctctccgtgcctcagtttccacaaagcaaggataacactgggttgttggaagaatcaatatagatattgtctggagggatgtaggtacagtgcatggcattgggtggcactcaaatgtcagctaataatattattattattctacgggaagaagacatcaggggaagttgcagagcagcctgtgggcggactctggaacaagaggctgaggcagtgcagcagagggtctcagacgtgagcgctctctgccccggaatgattgactgagcgcaaaggtctgcacgctttctctgtaaagggccagatggtaggaatttcaggctttgtggactgtatggtctcagtgacagctactcaaacctgcctctgtagcaagaaagcagctacatgcatagacagcacacacccaactgagagtggtgtgttcctatctaattgtgctactggacacccaaacttgagcttcccaccattccatgtgccgcaaattattctttttttttgagatggagtttcactcttgttgcccagcctggagtgcagtggctcgatctcgcctcaccacaacctctgcctcccaggttcaagcgattctcctgcctcagcctcctgagtagctgggattacaggcatgagccaccacgccagctaattttgtatttttagtagagacggggttttgccatgttggtcaggctggtccccaactcccttcctcaggtgatccgcctgcctcagcctcccaaagtgttgggattacaggcatgagccaccaagcctggccacaaattattcttaaacatttttttttcaaccatttaaaacatgaaaaccagtggggtgtggtggtgcacgcctggtatcccagcactttgggaggccagggtaggaggatggcttgagcccaggagtacaagaccagcctgggtaacatagcacaaccctgtctctacaaacaatcgacaacaaaaaaattagccaggagcagtgacacgtgcctgtggtctcagctactcaggaggttgaggcaggaggatcacttgagcctggaaaatcgagggctatagtcagctatgattgtgccactccactcctgcctgagcaacagggtgagaccctgtctcaaaaaaaaaaaaaaaaaaaaaaagtgaaaaccattcttagtggcaggctgtactgggcctctgggcaatcatttgcctagttctgatttaacaaactcttgtatggagtttactatgtaataggcattgttttaagcactttacaaatattcagccatctaatcttcacaacaaccctatgaggtaggctattattctccctttatagagtaaaaaaaaaaaaaaaggcacagagaggttaagtaacttgtccaaggtcacacagcaagtgagtggtagagtcatgatttgcacttgtgtggcctgggtttagagtccacactcttggttgctaggctgggccatgtctccctgtgcagatggggtgaaggaaagctgctttccttctactcccttatgcaaaataaggatgaaaatcctgccccacctctaagactatttggtgaacagggccaggtattctctgccctcataggacactctagtagagcagctgggtgctaataacagaaacacagaaaccacggagaatcacacctcccaaaaagtgccgtgtgtggaaagaacgtgcaagggtgggcagaaacgtcacgtaaactgagaagtgctgatggcaggaggtgtggatggcagtgggaaggaagaggggaagaaagagctggctggtgggctgactgctctcccctaccaccccctgcccatccagcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcaggtaagaggtgggagcacacctggcttcttcccaagcctccttggtctttctcacctggtttctgtcttagccatctcctcctgagcctccccgcagggtgggacgaggcctgagccacagggaacttccttcggttcgctgaacctaagttccctcccgcctttgcccatgctgggcctatcacctcaaaatcctcccttctgtgggacaaccccagcttgtccaagtccctggtatctgggggaggagttttcctgaaaccttctccctgctaccacccccagctggcctggctgctcctccgggctcaccatgccctggcctccctttcacaggactgtcagactgcatgtacagacattgtcttctcctgtctcccacgccaggctgtgggacacctggtgagcctggatcatctcattcatccctgtatctgcaggcccaacacggcccagctactgataattaatcataacaatcgcttctacttatggaaggctacggaacagcaggcactgtactgggcactttacatgcataaaactgacctgcatgtcaatgctaagagatagttcctgttgttatcccattttacagatgaggaaactgagccccagagaggttaaacagcttcttcaagggcacatggctagtaaacagaagagccagactcacccctaggctgtcgggctccagagccctgggattggaagatgaatgaagaaatggtggctccagggctccactcactgcagtttgttgctgggtctctttaggtctgaggcactgggactgtggggattgtgtcccatttatgcaggctgcattgtgccctggacctggtctatgacagatgtcactcctggttggcatctctagggcccacactggaggggcccctgtacagggtctgctggcctgtgcctctctccctcttacctggcagtgccagccagtgagaattcagggaatgaccatgaacttgggtcagtctgagatccttgcctggccactctggtgccataagaatttgggtgggatgcttaaccctgccaagccttggttttttcacctagaggtgagctatagcgcctccttgccagggctggtgagagatgggtgacattgtgcccagctgggcaccagaccagggccaggctccctctgggccgcctccaggtggggactgatggctgcagcccccaccacgcagccctcctccagctcactcacccccagctgctccccagctcactgcgcccctgcctcttgcctgcactcatgccacccctgcctgccacacctgttcctacctgcccccaactgcccacaggagctgacgtggccatttctgtctgccccatcatagcccccagcctcctcggtgggaggtctcatcagtgcccctcactccatcctgagaactccctatgggagggcctgggccagtgcctggaagagatcaggggccctacacactgttgaatgaatgaatgaatgagtgcgctccaattataaactcttagtctttgcccagttcttggattcgtctgattttttttttttaaatggggtctcgctcgtcgcctaggctggagtgcagtggcgcagtcttggctgacttatctgctcaccgcagcctccgcctcccaggttcaagtgatcctcccacctcagcttcccgagtaggcgggattacaggcatgtgccatcacgcctggctaacttttgtacttttagtagagatggagtttcaccatgttggccagactggtctcgaaatcctgacctcaggtgatccatctgcctcggcctcccgaagggctgggattacaggcgtgagccaacatgcgcagcccatctttctgatttcttagctacacctggtgtggctccctccttgggccagggtggagccctgaccatgtctgccctcccctctcccctctgccccttctgctctgtgctccttctcccgagtcccccagcccgtgtccctggcctctgtcttctctttctctccctcccacccctaacacctccctccactgtgggaacctgtaaaccccagggttgtgccccttcatggtcccccatccacccccgcaatgtctcatgctcgatatacaaaggccatggtgactttgggtgacatttgggtgctgtggaggctcagggtggaaatttccttccggccttgtgatttcaaccctcctcccccaccacatgcttggggctgttttgagcacagcaggttgccagctccatccacctcccggctaccctatccgagtagttggagttagggagaaccaggctggggtgagggcactcagcaggcccctgcagcaacagcagcagcaactctcattttctgagggggctacttactgtatgccagtcccttcatattcatctcagcaaacccaccgtccagtgcctccccaaccagttagaaaactcagttgcccacaggggctgggcaggaaggtgaggcaaaccttgggctgtccttggccggatctcctgcatctggctcccaagggaagccataaatccagatttttaaatgtaaacgcctgaattttaaatgttggtaatcaattcacttaaaaacatcaccaccaccaccaccaccaccaccaacaaaaaaacccgtagacttgtccctgttacaggcactaggaacacagcagggaacaatcaaaaagtccctggtctggccaggcaaggtggctcatgcctgtaatctcagtacttcaggaggccaaggcaggaggatcacttgagcccaggagttcgagactagcctgggcaacatagcaagacccccgtctctactaaaaaaataaaaaaaaaagtccctaccctcctgggttcagagtctggttggggaccccaggagctgggggctctggagatcaggagatcacagaaatggggagggacccagagagtggtggataggatgggaagtaaatgtctctagagagggaggccagggggtggagggcgcttcgtggaggaggtggcctttgagctaaggcctgagcactagagaagagctctctaggctgagggagcggcctgtgcaaaggcccaggggacctgaagggctcaaggggctgtagcagggggtggggaatgtggctggaaggaaccccatcaaggtcttggagcggcaggagagggggtgggagaaggcaggctccagatcagacagggcctggtaggctgtagcaaggactgtgggtttttgagcccccaaggaagtgatctgccaggttcaagggccagctctggctgctgatgggaaacagatttcagaggggtggggttgaagccaggacagatggaggctgttcacacccatccagatgggagtgaggggaggcttccatagcccaccatgcagcagcagggcagggtgacccttgcagaagtcatcttttgtttttgtttgtttttgagatggagtttggctctttcgcccaggctggagtgaagtgacgtgatttcggctcactgcaacctccgtagcctgggttcaagctattctcctgcctcagcctcccgagtagctgggattataggcacctgccaccatacccggctaatttttttttttgtattttgagtagagacagagtttcaccatgttggccaggctggtctcaaactcctgacctcaggcgatccacctgccttggcctcccaaagtgctgggattacaggcgtgagccaccctgcctggtccagaagtcatcttttgaagggagacaaggcaggaatgatggatgggtgtgtgatatgagagaaagatgggtccgaggctctgggcccaagcagctgggtggatggcagcaatgggaactgtgatgagcaggagaggttttggatgcgagatgggagtagaatcaagagttaagttggaggctgagcacggtggctcacacctgtaatctcagcgctttgcgaggctgaggtaggcagattctttgaggtcaggtgttcgagaccaacccaggcaacctggcgaaaccctgtctctacaaaaaattagcagggtgcggtggcctgtagtcccagctattcaggaggctgatgtgggaggatcacttgaggccgggaggcagaggtcacagtgagttgagggagtgacacagcactcttttgagaccctgtctcaaaaaaaaaaaaaaaaaagacagaagagacagggtctcactatgttgcccagtctggtcttgaactcctgggctcaagcgatcctacaaacttggcctcccaagtagacatctgttttatataattggctcctcccatctctggggtgattggggctgggtaggtagtgatgctattcttattcggcagaggggaaaatgaggcacatgcaggttaagtgacttgctcaaggtcacacagcagagctgggctagaatcttggtctcggctcctggcccagtgctctttcccatgtgtctgaatctgcatcttgggcaggggtccctgggccccactcctggacccccggactgacccccaccccatcttgtgcttagcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgtttta

SEQ ID NO: 13 - DNA, 3682 bp, Homo sapiens
  source 1..3682: mol_type = genomic DNA
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 14 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 15 - DNA, 34503 bp, Mus musculus
  source 1..34503: mol_type = genomic DNA
agtcaccagctagagcgcagctgaggcactagagctccaggcacaagggcgggagccaccgctgcccctatggcgcccgccgccctctgggtcgcgctggtcttcgaactgcagctgtgggccaccgggcacacagtgcccgcccaggtgggtgactcttggggtcacgggggacagctgcgcatcacaaagtgcccattccagctactgctactgcacaattccgggacagcatgagaggccatcacgtccccagcagacacgcgccacgcgcctctggggacccactggggacccgaggtctggcccgttgggatcctggggtggtactcggttcccgacagccttatgttccagaaatacccagtggctttctccgcgagcttgatcagggctcgaataacacccagcagggctcaggaacactgttcatccttgcgaaatcccttctgtcccgcggtctcgaggcgggaaggaccagggccactgccccattgcttgagcgggttgtgtcgtctgaagacttccttctctgcgccatggtctgtctctttcttagaaaccctgggattactagatgttggcggggccaggagtctaagtaagcgcctagtataagagaccgatggggagtgagtcacttctctgtcactagattgcttccatggatccgccttgctgtgttcaggggacgtttcagaatgttctcagtgtagtggcagagagggcagctaccccaaggaacctcgggaaaatggaccgattaaagatcccagtatccggtattcacattgcccccctcccccccccttcctgcctccattgatcgaagacagggtaaaaaaaggcagattacaggtacggaaaaatgaggaggctcagagaaattaccttttttgaaggatttaaagggagcttgctgcactgacatgcctttgcttggttccatgtatgctctctgcctcctgagcccaccttgatggacagggtgtgcttacctgactgcaggggagtagagtggaaggtggcttctgttgcgtccggaggtgatattcaatggtcctgggatgcaaaggaatggactcatccttggtgggtgtaggtcctcagggaggtgtcaaggatggactctgggtcaggacagtcccagttctgagggcggagtgtgaatgtgcatggttgtggtcaggagcctgagtggggattgtgctctgagctccaaggaaggataggtctttgggcactgagctggggtctgtccctggggacagacagagcacaggagcttctgtaccggatgggacacgcaaggctttgtgggatccacagtggtttggctgaaggtgtttccctcagacttccttccatacctgaccagtcccaggtcagcttagaccttctactactgttgcgtggactcttagctagtctgcctgtgttctaactcccacaaatcctcctttgacagagagaccttgctcaagcccagacacctctatctgggaagcctttgtagctcctagtggtggaggcagaacccttccttaccttcaaggtattccaggccagatggacatatctgtgttgcctgctcacatgaacctatgctctgtgaagctgatgactaggtgtctaccactgtgcgtgtatgacccctgcacacacattgtccatattacagatctctcaacacttctgagtcttttgtaaatgccaggtcaccagaaaggcctgtacacatccccccacctagacatgacctgctctatagagcacagattccatgctactttttattctctgtccctcaagggtagaagggacccttaacacagaactgtgcaagccttctcttgattgattccaaagaagctcctggtttgggacactcaagtgccagatactgtgttgggcactttgtcacagattagcactgaaacttcccttccagctgtttctacaaggtgggtaccgttgttccactgaaaggtgtggggatgggtgacaagctgtggcgtatgccaggaccacagaactgggaggtgatggggtgagatttgaaggcaagttgctagcctgactcccaacattctttctccagacattctcttacctgtattcccacttctatccctattgtgggactcagagatattgtgtgtagtgggtctgggcagcagagagagccctgaggatatttgagtaggttccaggggcagcaaacttactttacccttctattcagtctttcatttatttggcagataaataggtgtgaaacagggtaggtgagcacttgttctggtcttacttaacctgatatgtatggcctccctgtccctgggactctcaggactgaggactgctgtttgctgctcgagccagagtctgtagcatcccaggctgaccctggagtggattaggtacctgcagtactgctgaacctgagagctctttacttctttcctcctatctctgtcttgtgttcgttggctggcatccttctggggcatctgtattgtgactgaggatgtggcttggtattgggacctttagatggcggtggttaggtggtcaggtaccctgatatagagccatccatccatcaactctcttggcatttgtccatgcatgcttcacacactgcccactggacagctctaggaaactacctccagaaccccagaagccacatggccagaaggtcacacacttcagagacttgggttttgctcagaactgctcttgttacagttacatgggatacattcgcttttgtcgtggtaatagagaatttgcctggctttttttttttaatgatgatggtgtctcctgtagcccaggctggccttcaggtcactatgttgtttaggatgaccttgaagtccagatcttttttgcacatctccagagctaggattacagctgtgtgccatcaacacctgacgtatgtagctctggacttgtgtctctgcatgctgggcagcgctttaccagctgggctacatcctgaggccttctctggcatgcttgggttgaagaaagtcctggtccctggcccagtggcttcctgaaacatttgtccatctctaaggttgagtgacactttgacatgttttggcctttgctacccaatactagccagcgtagctttgtagggaaagagaagagttcacccagggtgtctttgtggggaagaagagagatgtttgccaccatgcctggtatatatatatatacatttcttgttgctcttgctgacagcttaatcacaacactcagggtggtacaggcaggattatcaattcaaggccatccttcggtacatgaagggtttgagataaccctggacttcatgaaaccaactaaaagaaaacaaaaaaactgaaaacatttgctaaatgtcagagatatttggctgacctttgttgtgactcgtgactccgttttacagccaaatgtgtcaggttggtgggcagatcggggttctggtggcccgcctctttcaaagtgtgtctgggcttgttcccactgctgtatcagatctgatgagctggagaccctctcctgaaagagctcacacttcctacctctactcatcctgggttatgatgatgccatcccagtggtggtggctcagggccttggagtgggcaaggagggaagttgagggttggcactgggagcagaagtgaggtgcatatgctatacccccaatttcagacaacatgagacaccagctcccttggtcagggcccaaggcctgatcacacagatgtcagccttcagccttcctctgttgtgatcagagcctccctcattcctttgtacagtggaaacacacccatggtgatggcaaggactaaggctttgaagtcactggtgtctgtcctgatttggttatattcagtgttcacagctaggaggaactcaggcacccatcagtgggagccataggtccccaaagcttaggcagcagatattcaattgccccaagttcctgtttgagtgaatttgctgcctggtacccccaaaccaacgttcttcctgtcaccttttcctgctgtacttcctgccccatatcccctggctgggcccctcagatcacttgtctgacttgggtctgggaccctgtgtaggaagcaaaagctatatggtcagggagtcaccatcttactctttcagtggccggtgctaaatgtcacttggggatgatgaggaatatgcatgatgaagttgaaaggagcacagaaggctctagctctgtgcttgtcaatttctaaatcatggccccttaggcatcatgggggggtcacctaagaccatcttgtatatcagatatttacattatgatttataacagtagcaaaattacagttataaagtggcaaaaaatataactttatggtttggggtcaccacaacatgaggaatggtattaaagggtcacagcattaagaagattgagaacccctattctagctcatccctacatgccaggaggtctctctcttaaatctcttccatctgcatttgtgtatatttatgagtgtgagagagagagagacacagagatggagagacagagagagacagagaaagatcttgcttacttaagacccaagatgggaaagccatcttctgaactgaagctgtctaattccctatgcccaagtgacttctaagacagtacaacctgggccagacggtggtggcgcacgcctttgatcccagcacttgggagggagaggcagatggatctctgagttcgaggccagcctggtctacagagtgagttccaggacagccagggctatacagagaagctctgtctcggaaaaaaaaaaaacaaaaacaaaaacaaacaaacaaaaacaaaaacaaaaaaagacagtacaaccttgaggaggaatcacagcctgagtgttagtagaacagagtttaatggcttgctgtgtgtcctttccatttatttaatctctctgagcctaagtcttccagcaacactaatctcaatgttctcccagaaggagtggagctggcagctacatgagccatgagaaaaaaagagctgatgtctttaccccttccttccttccttcctccctccctccctccctccctccctccctccctccctccctccctccctcccttccttccttccttccttccttcctttttgtatgtatgagcagtgtgcctccacatgggtagggatgtttgtctgtatgtaggtgcatgtctctgtctgaattgatattagatgtcttccttgaccactctacagtttacttgattgaggtgagtctctcactgagcttggagttggtctagctagccagcttgttctgggatctctgtttctacaccctaccccagcccctgtgctgggtttatagttagctgcctcatttatgtgggctctggagggtcctagctgtggtccttaggcttgtaggcaagtgttgttccccggagccatctctccagtccttcttgatcattttattcccatttcttttggttagaattatttccctgtcttaggcccccttggcaagatttaatggctgcccctcaggttgctcgaagctggggtgctgctgtctcaggcaggcctggaagtcttgggtttcctgctcctgtcccctagtgcctttgatatgtcaacagtgaggcatgcccccagatgctgaggaagtcagacttgcgtaacagccagcatgaccagctagtagcctccacgctcaagtaagcccaagtgggaagggtgtttcctcaaagaagctttttgtcactcattagaggggacgccttatttggtactgattctggcctgacagtatgtctctttgtcctaggatgcagggcaggggctttggggtttgggacccacctcttatgtaggacaggggctctgcagctctgggacctaccactctgatacctacacacatctttttccaccacggtactttgtgatgtcatagctgacagtgacaccattgtggtttggactgcttggctgtgagcaagagaagaggaagcaggcttagcttaccagcaggaggttgaggaagagagtgtccttgcacccccagtgtgagccatgctagggctcaggtgtgaaggtagctggtgtgttccaacctgtagctctgtctgacccttgccagcttgaataggatgatggaagactgactgacatcttgaaggtcatccacacccatcctgtttcttttgggttttttgtgtgtgtgtgtgtgtgtgtgtgtgtgtgtgtgtgtgtgtgtgtgtgtgtgtgtgtgtgatgttttgttctattgaaacagggtcttgctttgtagcttttggtcagcctggaacttgctttgtacaccaggctggccttgaactcatagagatccacaaatctctggttccccagtgctgggattaaaggtttatgccactacaccaggctccatttcttctttatggaggtggaggggttaggtttgtggatgctatgttgttcctcatttaagaaggcctgcacccagcccacccgttcagtgtgggaggctgcaagaggtcacacagtctgagtaggtgtgctctcctgtttaaactggaaagtgacagcatccagtgacctgctcaggggtgctgggtccttgagaggtcttgggaaccatactgttctctttgggagcctgggcctggggtgacccagcccctctgtcaagacacgaggaagacttcccactcaccaccactcaaggtgtttgtcagggaggccagtgcaggttgtctggttttgatcttgggccatagcaacctggagttcagaaaactgagtcagtctgaagttgaaaaccgagtgagaagagacttgcgggtagtcttgcaggccatgtacaaagacatctttttttgtcctctggggacagggcagaggctgtagatcctgagttctgacgtctggtatgacttccactttgggctggaccaagttgggcctccagttcccttgcctgttctgaatctccattgctccaagtctgacttctcaaagccagcattggaatccaggcctgcagctgttggacagtgggtgggaagctcagtagcttggagcagaggatggcgtgtaactggactggacagtggatctctgaggaagggctctggaatgttgactcgagtttcaggacccctgccaatgccctagctatgccctgtgcctggcctcccactgctgaccgacagcccagcttctggctactgctcacagagccaaggttcttaatgaggctgtgaccagctgcttgctctctggctgcctatgcctctggccatgagcctgggtcctgctgcttcctgctttctctggcaggagtccagccctctccagtcttagcccctcaggcctccactcttgcctctcggtggcttggtcctgtggtcacatcctcaccttgtctagtcacccatatgattctccctgaattctgccctcctgcaagagttcacagggaacttttctggccttgagtcccatagcatggcagactagaaatgagtggatgccaggtagaggctggagggactgggagcaggctagacccagtatctgggcaggaagtgtgctagaatcaacaaagactcatgaggctgtgtatctgcagccagtttgtgaaagaagtaatagcaactgcattaaacaattattggggccagttgtggtggattgaacaggcaatcccttgggtggtggaagcaggaagatggagagagttccaggccagcactgtctcaaaaagaaaatgtagtttgcaaaacaccttattgtgtgttatgttatgtaatttttttttaccagacccagacagtatgttgtcagattcattctatggagaaaagggttgggggaggaataatctttttttttatttgaaaatacagtgaacagggctggtgctggggcaatggttcaggagttagggacagaaactgctcttacagaggaccttggtttaatgcccaccactgaaacagcggttcattaccatctggaactgatatcctggggcaccagccgcacacacgtacatttaagaaaaacactcatacacataaaattaaaaataatagcaacaacaatagatgggctagggaggtcaggatcacttgctgctcttgcagaatccggggttctactcccaacacctacatggcaattaaatgtctgtaactccagttccaggggatcggttgcccattctggcctcttcaggcaccaggcacatacatcgcgcatagacaaacatgtagacaaagcattcatacaggtaaaaatgaatttatcttttaaaatgtaacaatgtaactaacaatgtaaatgtaaaaatgtaacaaatataaaagcaaaaatgcaacaaacaggctggggatacaattgtgcaagaaagaatttattacattgtgtaataaattaataatccaactgattaattaaaaaataaagctccaaagaaaaataattccttcaagatgcctgctaattagggatttaggctgagttcaaagccatacttccatacttttgtctgttgtcaactaacacactcttgccataagaagatgaggcgacaaggagccatccatagtttggaggcaaggagggagcctggggcagagagtaggtggactgatcatctggcctttgtgtggaggttcagaaagtctagagtcagggaggctcactgggaggctgcagtggtgactcaggtgaagtagggaggacagcagaggagggacggagagggggaggaccaagcgatgagaaagctggaggtaaggcatattttcccgtgtccccatcttccatagaggtttactgctcatcctgagctggttgacttgttttggtcattaaattgcctgaccttgaaactgctatgtatccgagggtgaacttctgatcctcctggttctctgcctcagttgctggaattacagatatgcaccaccatactgtctgtttgtttgtttgggttttttgtttgtttttgtttgtttttgtttgtttgttttaatgcagcgctaggcatggaaccaggcccttcaggtgtgttagacatcctctctaccaactaatcttcattctagccttctctgggtcacagtcagccacaggatcagccttgccataatcaccctggtcgcctactctgactggagtggatttgaaactgagctgaggtctgtaatgtctcctgagagccaggcatgtgccatgattaacccagcaggtgaatgagaagaaccggatgctgcacatggcttcggcaggtgtctaacaaatagtttcagtgataatcaacaaaagcaaaaggtctaatttcagagtgggtctgatgcactgaatgtaagaagcccttccattttcagccagtggggcagcaggccaatactcttgccagaggagatgagagggtcaggccttcttgtgcatggtactactgttcaccccaaccaaagcccagcttccatccataggctgcagaggctaccacccagatgtgatggtggttggttgtggccactacctgggctgtgatctgaaggctgcccttcagtttctggggatattgccttggccatttcttgaccagttctcatgtgagccaaacacagctcagtgaataggaaattagaactttcacttttgttgattagcgttgcatctatttattagacacctaccccagccatgtgcagcacccagttcttctcattcacctgcttggttaatcatgacactatgtatggtgggcatggctccgtttccattttgtagatgccaaaaccaagatggaagaataagagaaggaaagtcaaaccaggttctggctcaaagggagatcacagtagggtgtcagggaacacacaagttgaacagagttaactcaaatatgctctaacctgggaaatatacaaacagtcctaggttgtcagaggagccttctggagggatgacttccatctggggttcaggggtttatatgagaaagaggtgatggtgggatgagtctgaagaagctggggggtctgctctgcagaatacgtggtcagaggtagcagcaggagtcagggtgcagaagaggagtacgggagtcgtgtgctttgcgtcaggtggcagatgaaagctggcagcagaagctggagaagtttcattccagcctgaggacagctgaataaggagggccagtcctggaagtaggaagatggcttaggtctgtctgcaaagggaaatatgctagacaccaacagtgaggaaactagctggcaggagttgtccaggtattaaggagagccagttggagatgaagaaacccatatcaaagggcctcagggtaatagcagagagggtgaggcattctctgtcgttgaactgagtattatttgggggcagagcccttggcatgtagcctggagcctagccctaagaacaggatcccagcgtgggtttcctgtctgctgcagctcacttctaaagcacatcacaggcagcagtttgaaggagggatgtctcacccctgggccatctacctgtgatgtggttggtccttcctgacgccaatgctctttgtttccatgatggttggcaggcctggtcgcctaggccttgcgtcgctatgacagcctagcttgatgtggctgtaccgccatagtccatgggctcctataaacctccaagatcaagacttaaatgcaccccccccacaccttccatgcaaatgatgtggtctctctagtgcttgaagatagagcttccggctgagtggagggtccttgggcagacacctctgtctcatttgacctgggatgtggtggcccttttgcctggaaacatcatcaaactcttcaccactctgtaaagccagacaaaaatcatagggcatgaaatatctcagtcagtaaagtagaccagcacacgcttgggatcacagcactggggaagtggacaggcagatccccgggttcactcagtggccagtctagacaaattagtgaggcccgggccagtgagagaccttgtattgaaaccgagatggacaatgcactaagaagagctgacatctgacgtgcacatgcgcactcgcaagcacacacacacacacacacacacacacacacacacacacacacacacacacacttgtacccccatatgcacacacaggacagctgtgtgttcttgcacatacttatacatgtgtacctgcatacacacagaaaggtacacatgttcatgtgcacctgaacacacacaaacagttgtgcacacacacctctactcagacacacaaagaacacaagttataaaactgactgatgtagctgtgatgagttcccagtctgcacctacactgtatctcctggatctttcacttctacagtgatggtgaaatggactctgtcttagttactttttgatttctgtgaagagacaccatgactaagacaattcataaaagaaagcatttgactgggggctagcttacagtttcagagggtgagtccatggctgtcgtggcagggagcatgacaggcaggcaggtagacatggcgcttgaacaatagctgagaacttacatctgattctcaagcaggaggttgaggaagggagaatgggaaggagggagacacctcctggcatgggcttttgaaaccttgaaacccaccccccatgtcacacctccttcaacaaggccacacctccaaatccttcccagacaagagacaaaacttttgaatgtataagtttatggaagctattcttatccaagccaccgaaggcttgtactgacttcatttgtttggttcagagaaaaaggtggggaaatatatacaaatgctccagaatggcacttcacacagtaggtactttttctggatataagccttactagtctttctagcttatataaatggtgattctagtgggtatatactctttaccacaagctgggagttcttccaatgccaccttccctatcttgctctatacctgcctcagtgatagatctatacctagggtctattattctttcacctctcagtctgtccatctctacatccatctatctttccatctatctatttacccatccatctacccactcatcatttcatccatccatccatccatccatccatacatccatccatccatacatccatccatacatacatacatacatcctttctcccatctctccatccattcattcctctttccatttctctttccctccctctatttctgcatccatccatctccatctctccctccctccaatttttcatccatccacttctccatctatcctcttttcagtctttgcctaccaggtacagtaggtggttcattcttaccataggtttccagttctgacctggagccttgtttgctttttgttgttttgcttgtatgtttgtttgtttctttgtttctagacagagtttctctatgtagccctggctgtcctagaactctctatgtagaccagattagccttgaacccacggagaacctcaggcctccaccaacttagctctgggattaaaggcatatgccactccactactccctgctggaggctttatcttctgagcaactggctcacacaaacatgctgacagcatcaagtgaagttggattcatttcctcatttctcagtgtctgatctccatgcactgggggtgggggtgaatctgtgtctctctaatttaaagtaaaagactagtcctccccagccttgggaatctccggacttgtgaaccctctctagtgcccccattcccacccccatccctctgcagtccctgtagtggccctgaggcccagcccccaagactatgttttggggctttgggtacctccctttgcaggagtgaccttttgttctgcattacgcaggggctttctgttcctcattgtttagtcctgacagagattgtggtttttttttctttcttctgtccaagggaataaacaggcctggaggttgttgagggaggcatgggcccctttgccagctgctgggactgggattacacatagcagcaggagaaaggaccagaaagtttattcttagtgaagctggggacaaggaggcccccttgatgatgggtggatcagtgagacagtccaacttggctgagtcaaggccctttcccatcaggccctcttcgtctggaaggaagttgtttctagccaaaacagtgtttctctgggccactagaagctgcccaggccacagctcagttaccaccacccaagggatatacctaccccagggctggcagggcaagtgttcccatgagatgcacacaccgacagcccgtgtgtggagccggaggctgctgtttgaggaagtagaggagggtgaagagaaggttcttagttaatgagacaaatagtttccccaggaaggctagtctgggagttttattggggacactcaggccttctgtgtcagttagcattctttggagtgatggatacaaatgagctatgacactgatttctggggtccaaggctcccctaattctgctgtgtaccttcctcactcctttctcccaacttaccttactttctccatgggataggatactcagaccacattgagtagagcagaggagtctctaatccccaggaagagttctttcttattttatctttcctctttatgagcagtgtggctccccagcccagagactctccccccttagatgtgaaagggtcagccagccattgatgtaccctttgatgtgtttgttacattagcagttctcatctgaacacaacacactgggtccgagcggttccacgtgtgagatgtttattgggaggggggcgcaggggagaaggtgacagcagaaagagaaggggaaaaagagagagagggcctccttagaagtgggaacaaaacacccttggaaggagttacagagacaaagtttggagctgagattaaagggtggaccatgtagagactgccttatccagggatccaccccataatcagcatccaaacgctgacaccattgcatacactagcaagattttatcgaaaggacccagatgtagctgtctcttgtgagactatgccggggcctagcaaacacagaagtggatgcccacagtcagctaatggatggatcacagggctcccaatggaggagctagagaaagtacccaaggagctaaagggatctgcaaccctataggtggatcaacattatgaactaaccagtaccccggagctcttgactctagctgcacatgtatcaaaagatggcctagtcggccatcactggaaagagaggcccattggacacacaaactttatatgccccagaacaggggaatgccagggccaaaaagggggagtgggcaggtaggggagtgggggtgggtgggtatgggggacttttggtatagcattggaaatgtaaatgaactaaatacctaataaaaaatggaaaaaaaaaaaggaaaaaaaaagagatattaaggaaaagtaaaaaaaaaaaaagagagagagagagggagggctgttccccttatttatatgaaaaaaatgacgtaacacaggtaaaggtgggaggtgagccaagtggattctgggaatatgctgcaggtcgcgctgtcacctatgtgatgtcataggtttgggaggtcctgatgcaaagacccttgaccttgagaacagtcagtgaggtcccagacagtgtggaacacccttatttgtgtgctcagactcaactggttgttttgtcaagaaatggaacggggaagtgccaggatattgtgtgtaggcatgtgcgcatgcgcttggatgtgtgtgcagctgtccagagggagctgcagtggctttttcagactgggtactcagcatggttctgcgacttccctcacccagctgtctgcatgaccttcctgaaaaggattgccttgatggaggaactcagagataggccagggcagcagatgcatctatgttcaagcttcttggccctggtgcccagggggaggctctcgcctcaaggaaatgacattagaattattaccttactcagggtagcaactattgattactcttttctgtctggctctggatagggtagctcgcactgcagcctccaatgggccctcatctggggttactgggtgagcatgaagacaaggcagggggctggctgtattgggttgcttatccagggcacggtgtgcacagtcgcctgaagagggaagcactctgtgtggacttttggccaaaggcgcaagaggcagctagggtactgtggcaggtccagaagagccaaagactgtccacagcatagaaccccagtcccttgagtcatggcttctggtggctttcagctgttatcactgagtggctgagcaaagagaaacatgataggctcagaggtcactcagtgcacagcactaggcaaggtagggtgatgctctccagacacagctggggatgggaaggggaaagacagaaagcagagccactgcccacagatcatgggagtttgtgcagtctatacagaccaaatgagcctaatagtaaggtcggcatcttctctgattggcttgtttgcagtggcttttcagagactgatccatgcctttgctgtgctaattgttggatattttatgtatctctcttgctcccatagaattgagtcaggaaccgatgtccagagagatagggacacttgattgactatgaccatacagcttctggatggaagaataagaaaagaagaccacatctctctaactctccacctttatctgaggagtcaggggattcatttgggttactggtttaataaacatttattaagctcctgctgaccatatgacggcagctgctggtacaatcaagagtgcggagcagccttcctctttaacctagaaccaccccttggtcatttccaggtgacatcagcagaggctgcaagggtgttgagtgtgtgttcacaccctctttgctcttgtagatccactcccatgtttgctcatgagttcacatttgtgcagaaatggctaattcatgcttgtccctaaagtttccaattgctgttccaccttctggaagaaaggctggggcttaggcaggtctctgaggtactgggatagacccagtggtgacagttgctgctgctgtacacttctgagaactgaggacctcttggagatctgagggagggctctgtgtttctcacagttggttctgtgccatgttgggtgtttttttgttttttgttttttctcctccccatttgtctccctttcataattatttatttaattaaaaaatgtgagtactcctgagtatatgtatgtgaaccacacacattcaggagtatgcagaacttagaagggagcgtcaggttacctggaactggagtgacagacagttgtgagctgccatgttggtgctgggaactgatcccaagttgtctccaagaatagtaagtgcccctaaccactgcgctatctctggagctcctcctccccatcctccccatcgctccctgctttcctggtctctccctggaagtgcttagcccagtcattgaaatcatggcctagagagacagacaggacccaggctcaaatctctgtcactgccttaggtgggtaacctgtgctcgttatgcctgtttcctttctatacaatggtcggagtccaacatggctggttgttagagaactgaatgcaataatacatccatctcaggtccaggctctggaagcacagcctgaggcatggcttcttgtaacaggagaaggctctcaggcaagagggtggggtggcaggatgtggcagcagggtcacatgttttccagccaaaacctatgagtgttggtcttgggttgaggccaaggtactgcctttgagttggagacttgtcggccagtgctgaggctattctcctggcagttggagtctgagcatggctaggagcccccactggtccaaaccttctaagcccataggtgtaaggcagcactgatgtgaggaggtgcacaggtttgttttcattctgtattaacttcctgaggcagctctgccatggcctgtgtcagaagcttgggtctagaggtggcgcagccattatctgtgtcataggtggcagcatatggacacatgtatgtacacctgtgtgcatatgcatggatgtgagtgcaaggacacacggtgccaattcagaacaatgacatgaaggtcagaaggggccatgtctatattcagagagaacccagaggaggaagagcctacattaggcccaggagagtagagggtacttctgggagaaggaagacctcagccacaaggacagagggacagatggacagacatcctaaaaagagaagctttgtgtggaggtccttgtcacagtgagtcaagccactgtcttaaaaaaactacatcttctcagagccttgctgggtctcacccagcaggcaggagggaagccctaaagtaacccacttcctggcccagcaaactgcagacacaagcgtgccacgctgaagaggaaggccaagaagggaaaaagcctgggctctactctctcaagccttcctcgtcctccaacgcaaaaacccatccatgaggcctggtgacaggtatgctggagcccagagtcatactgatggctgcctcccttgttcccttccaggttgtcttgacaccctacaaaccggaacctgggtacgagtgccagatctcacaggaatactatgacaggaaggctcagatgtgctgtgctaagtgtcctcctggtgagaggcagctgctggggctttggaaggtggtgccatggagggagtgcttgtctgggaatgagggccttcagctctcactggctgctttatacatgctagggttcatgattcatcttgccctgggctcttgcatgtgctgtgccctccactgagcacacttctcagtgtcttctcctggttactgcctacctacattttgaccctatcctcttgtccaggaagcctcctcaatactgcaatgatgtcctagtacttttcatagccccactatgtgctatgtctacccctattgagtagtgagtgtagcttggtccctgtggagttccaagcacacggctggcctatattgatgtcctgtacttaattgtcaagtaaatgaatggatagccatatcatagatggcggatctgagccctggcctcattggcgaggactgagtagctggcccccagtgctgagtagacaatcaagtgtatgcattcaattaagtcgtatttataatgcatctactctgtgctcaatctgctgagaggaaagccacacaaacacgggatacagcaggctgggaagtaagtgcaaagccctaaagcaggggcctgttttaatgggtccaaagaagcttgaggctggtgtttgttgcccagagtgaaagaagagtgtgatggaagatgggtatgaaggggtgagggttggtgagatggctctgtatgtaaaggcacttgctgtgcaagcctgagaacctgaaccaatggtggaaggagaaaacaggttacacacacacaccaccaccaccaccaccaccaccaccatcgtaataatatacatttgattaagcgagatgagtggtgtcaccatgaagaattcagatttcatttactggaggctttgagtcctgtgcttgtgacccagtgggcaggaacacagggcaatgtcatcctccacagtcacagccaggggacagtggcttttctagtgacgagcagagtgtctttacgaagttacaagcttagagtagcaggaacagaggagacgagctggttccagaagagaaaaccttaaagatttgtcctgttactcttctatcctccaggctcttaggagccctatgcccttggcactgacaggctaccagacatgacgcccttatagccacttatatttagtacatcaggatagagaattttaaccaactatcaagaagacaggagctagcaaacccattctcccttaagtattcttgactgttaataggtttttcgagtggtggtgtttcaactacctggagagagcagagggaagatatgttcagaggtagagagatagctagggccaccagacatgcttcatcccaggggcttcctggcaagggtcttctgctgagacctctggtccttgcttcctcaggccaatatgtgaaacatttctgcaacaagacctcggacaccgtgtgtgcggactgtgaggcaagcatgtatacccaggtctggaaccagtttcgtacatgtttgagctgcagttcttcctgtaccactggtgagtggcatgagcactgtgatcccaacatcttccccagtcactcgccgtccagcaagtaaggtggatggggttaccctgggccagccgttttcgtcttccaggacttgagttgcgggagactgctttttttaaaatatttttttattacatattttcctcaattacatttccaatgctatcccaaaagtcccccacaccctcccccccactcccctacccacccattcccattttttggccctggtgttcccctgtactggggcatataaagtttgcctgtccaatgggcctctctttccagtgatggccaactaggccatcctttgatacatatgcagctagagttaagagctccggggtactggttagttcataatgttgcacctacagggttgcagatctctttagctccttggatactttctctagctcctcccttgggggccctgtgatctatccaatagttgactgtgagcatccacttctgtgtttgctaggccccagcctagtctcacaagagatggctatatcacggtccttgcaccaaacgcttgctagtgtatgcaatggtgtcatcgtttggaggctaattatgggatggatccctggatatggcagtctctagatggtccatccttttggggagactgctttttttcaacttgtatgtagttatgtgattggaacctgggacttcatgcaggtgatgcaaacacttttttttttttttttggttttttgagacagggtttctctgtgtagccctggctgtcctggaactcactttgtagaccaggctggcctcgaactcagaaatctgcctgcctctgcctcccaagtgctgggattaaaggcgtgcgccaccacgcctggctgatgcaaacactttttaacagaactacatcaatccccagatattttttttattaaaggtgtattaattaattaattaattaattaattaattaacttacttattttggtttttcaagacagcatttctctgtgtatcctcggctgtcctggaagtcactctgtagaccaggctggcctcgaactcagagatctgcctgactctgtatcccaagtgctaggactaaaggcgtgcgccgccccaacccaataagatttatttatttttattgtatgtatatgggtatttttactggcatgtagtcaggatctattttgccttgagtcaggatctctctaggtagctcagaatggctttgaagttatgaactgcctaactcagcttcctgagtagctgagaatacagacctgaactcccagaaaatgttttcagttcagatttttttgtttgtttggtttttgttgttattgtatttgtttatttggttttggtttttataagatagggatgtgtgtgtgtgtgtgtgtgtgtgtgtgtgtgtgtgtgtatatagtcttggctgttttgaaactgaactaactctgtagaccaggctggccttgaactcatagagctccaactgcctctgcctcctgagtgctgggattagagatgtatgccacccctgttttttgttttttgggttttgttttttttttttttttgagatggtttttttgctaagttgcccaggttggcctagaactttctataaggcccaggctaactttaaatttacattcatccttctgcctcggtttccccagtgttgagattacaaatgtgtgacacctgcctggacagttccagatgacattttccttctagatcctaccaaatcttcattggtagtggggaggtgaggtcagagttgagaatgtagagcctagagaaacagatgtgtatattcactctgtggtggagttcagagtacagacaacaacagcccaagaggcaaggtgttcaagggtcctttgggcctgtgctgagggccctaccagctctggcaggaagagcagaatgggtgagtggaagtagagatgaccctggctgtctttctcacctctagaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagtaaggatccctcttcctctccctgggaaaggatgtgaacatccctgagatgtcacaggcacttacctctctaagccttacctctaagcctccagtagctgccacatgcattacatgtctcctggcacccctggttacaccaagcccttagctagtaggaaatcaggtaggacaggaaggtaaccctggacagcaagtccatgtacatgtatggtcatgtgtgtccatgcatgcatgcatgcgcgcgcgcgcgcgcgcgcgtgtgtgtgtgtgtgtgtgtgtgtgtgtgtgtgtgtgtgtgtgtgtgtgttttaagggagaggattacaaagtggcctcccttgtggccccactgttaagagactccccacctggctacatgaagtgttcatattgtcgacaagctctggattttctcttcaaggagccccaaatggaaatgtgctatgcaaggcctgtgccccagggacgttctctgacaccacatcatccactgatgtgtgcaggccccaccgcatgtgagtgttgacttcctggcttctgaaggagcagggagggtgaccgcctaggggaactgggtcccaggatataggacagcttgtcactctttgtctcttgacttgggttctgggacagacaatggatgacttgagcaggaacctgacatagaggcaagattggatagagtgacactgggtggaagttgaggagtatatgagggggtagtgctaagaagcccatactcattgcttggctgttaggatccttgtcctcaggcctggctccctgaaattctgtggtcttctatcttctgtggtctcctgattacaatcttcctcccccagctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattggtaagtcctcagtctcaagagtgacccaggcaggcttggaagtgtgcagtctgagctactggggaagctgagacaagagaattgcaagttcaaggccaacctgggctatagagtaagtttgaggtcaatctgggcaacttagtcagaccctgtctcaaaatagtacttttaatgggagctgaggctgtagcccattggtagaatgtctgactagtttggggttaaatctgtaacaaaagtaaattgaaacataaaagggaggtgatcccccacccccaccccctgtctctcttgcctccctttgggcagaactggggcctggtctctgtcttttagaacagatgcaagtgtagaagagttgagtctccaaagatgtgatattgccatagggaatccctgggtggcatgaatgtgaacataggttctggttgctagttatgatgaacccgtttgtctcacagactatggactgctggtgtctctggcttccctctggggttaggatggtaccaagcccaaggccctacactgtagccttctgactgcatgtggtttgcagatgtgatctttggtttgaccctgaatttgagggcctaaggctggacttgatctcagcatcagcaccagccaccctggaacctttgtttctgagtaccctgccgttttcctaggtctgattgttggagtgacatcactgggtctgctgatgttaggactggtgaactgcatcatcctggtgcagaggaaaagtaaggttctgctctcgtcctgtttcccgccccacgtccctaccctaacactttctcggagccttgggtggcaggcctcgttcagagctctgcactatcatagactcgggtggatctggagactgtagcctcttgctctccaggaacttagagcctggtgtagaaggtacatactctaacctggctaccagaaggtagtgtcaaggctattccaggggattgaagttcaggatttttatacaggaagcctggagtctctcggattctgtgagctggcctgggtctcagtgggtggagtgtgttgaggcagggctacaacacaggctgtactggaggatggctggggcaaaggggtagtggctgaaagtagcatgggctcatccgtgaacatgacaggcagctactccctgggctgggtccactctcctggctctaccctagccatgctgtgcccattaagacagctgttctgaagaagtcctgcctctgacttgttcccctctcttcattgtagagaagccctcctgcctacaaagagatgccaaggtggtgagtatccctctgcggtcctcctcccccttctctcctccagctctccctcttcctcctcctctttctcctcctcttccagttcctttactagggcatcataagcaacatcatataagcaggatctaatgtatatggtggtgcatacctgccatcccaatagttaggggcaggtagaggctggagggtcagagtgtaaatactccctcagagccagctgatgtttagttccattacagttgggaagaaaccggcaaactctcccctagctgggcatagccaggctctttccctaggctatgtccaactcctcactggtcctatgcataaggcaccttggctcctcattatatagatgaggaaatgtaggcttaacagatacagtcacccttgaaaggttgtgttgtgtcctggtggtcaaaccctctgcgcatgtgcatccggcagctacacccaaagctgtctcaggctggcctctcctagcagggactgctcgccaagactggtcaccaggatttggagaacctagatgcccccctttgtccagccctgtgccctgtcagctacctgccccagggccctactctggagcccagtggacggtgtcctcatgtgctagagtgaggaggctgtcaggcaggagcagcagtgactcagcttctactctgaggactggggaagatgaggttatctggaaaaacagcacgtgaattcatctacacctcccccagtcatgcgcatggttcatgtttagtcgccggcagttactggtggtggtggaggaaggacgtgccagcactgtctgagggcttattcttccagcttgactgtgacacaggctatgacacaggctgtggcacaggctgtgacagaggttattgcccccaacgttagacagagtctgggattatatggctcagacactcttaagtaattgtcctgtgtcctagctgtgtcctagacacagctaatgggtatggcctggaacacaaacttagcatacaattactacccagcatacaattactgtgccactcttaggcatgcccacatgagcctgcacatataaatacccttagctcagaggccgacagaagccagccatgcttctgccagtcccagttccatataacaggaccagacccagaaccataggaggtatcaggcaagctaagattttttggtgttcattcagtggggtcctagccagagtcacatgactctgatagtgtctgtacttgaacactgtagcttgtggctttgaagaagctagctgtgacctttggggaggaggggaagatcgggttctgtctggctctagccacaagcagaaagggggaggcccagacagaaaagctccttctctggggctggtgtggtgtgacatttatgagtgcctctgtagagttacagggtaaggccagggccaaggtcagaccgtgagttcaggatgtttttcctctcttccattttggggtacctagctgggaggaccccaggggagtcaggaggatgcagtgggcatgctcccacatcctagactgggtttgcttactgtggccatcagccagctctgtaactctgagctctggattcatcttctgtgggcttttgtggcctcatgaagacagcttgtataagatccttccagtccagagtcagaagctgtggactgtgcaggaaacaaggcctcaaggccatgtatcatgacatacctgggcatctaagaagacatagcaacagtgggcaggacctggagatggaagttttagtgactctctagagagcaccagccacaggaaggatagcccaactattgtttcagcttagactacacctggcattgtgcctccctggaaaagtgtggccacttttccagctgctgtcaacagacaatggccttggaatagatgtccttagcataagttgccatgaacagggaatttgtcttaagcctcaagagctgaaccccagacacccagaagaggcatggtgagtttttagctcgactgcactgtatccaaaccatggcctcaggcagaaagttccgtttccatatttacagagctaggatggcactaggctatattgagtgtctagaagagctgagaccaatgtgactcaggcacaggatttggcactaggtgctcatgtcacatacagagtaagaagccacagggaagccacacgacatcatctgggtgaactgtgggaacgggcagctgagaagaccagtgaggaaccctgtgagtggccctgtgagtggccctgaggaaaggaccccatgggccagggtgattcattgagcagaaggaatgaagagtaaaggtgttaagcttcaagcaccaaggctggcctctgggacagctactctgatctgccacccaccatcaggaagtctgtgctttgcaaaacattgatgacagctcaggtgcatcaaacttgtttatttacgaacactaaaacttgacctttagatttgaggcattttgtattttagacacggacttatgtagcctgtattgaccctgaacactaactgaggatggctttgaatttatgatcctcctgcctcaacctccccagtgctgggattactacatacaccaccacaccctgcttgtgtgatgctggggactgaacccctgagctctaggcagagtctttagtcccttctgttcttgcagaaaacatgatttcagttctcagcaccaagagaggttaggtaactttttcaaggtcatctagccagtgagtggtagagctatgaactttagactcaggtgtctggttcagaattcatctcttagctgccaggctggtgacacatctccctatgctccaaaaagtcactttttccatttccttgtgaggactgagattgaaaacctttggtgccctgtgtggaaacgtgtggtgtgggcagaaatgtgtaagctgtggagtgtgggtgacaggggagactaggagagcaaaggcacttgcagatggattaatattctctcttttcctcccagcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcaggtgagaggctgattccctcctgctctggacagatagctggccctccacctactggttgaaaataacccaagaggaaaatgcctttaacacttggtggctatgacaacggacagaatggctctgatctgctgcccagtgtcttgcgagaggatcctattgcaattcaatagttccgaaaaaggtccaggctcacagtgctaaacacagctcccatggaatgcgtgtatcatgttcccaccaacgtcaagttgcaaaacctcgagtcagacttctgtaggtaaagtcaggttcatgtgtatcccttgcgtaatctctgctggtgtcctggagctgccctgtgaagccacccttagctgagcctggctgacttcctagttctggctcatgtgcagtccagccacggtaggttcagcgccacctgtgttggttatctcactagctcattcgtcttcagacacccctgctttgtctttcaaggcttccatatcaggtggggagtaatcctgaacccctccacgttgtcatctctggccagcttggcctttgttcagggatccccaaggtcatgccgcctttccacaggattatctgtcttctgcctttcgatggcctgtgtgatttccccaaagccacgttctgcttatatccctaggcccagcactggtctggctagattgtttgtttgtttgtgagacaaggtttctctgtgtagcctaggctgtcctggaacttgctttgtagaccaggctggcctggaactcagagctccctctgcctttgcctcctcagtactgggattaaaggcatgtgccaccatccctcattggcctgtccagttttataagctaatacttcctagggcattgtaacacaccagtctttgtcctgggtgctttacatgaaccaaacctgcctatcaatactaagagatagttactattactaccccattttccagatgaggaaattgaggtccaggacaccttgttaagaggacatagctagtgcacccaagctccaggagtcatcagctctgcagtatgagcttcagagccctggaaatgggagatgaatgaaatagttccttcagggttctttaaaaatgttttgtgtatgggtattttacctgcatgcatttccatgtactgtaggtgtgcatggtgtctatagaggccagaagagggtgtcagatctcctagaaatggagttacagatggttgtgaagtgccatgtgggtgttgtgaatctaacctaactcctctgaaggagcaacaagtgctcttagctactgagccacctctccagacccattcttctgggggttctgctcactgtagctcactgatgatgatcactttgagtcaaaggcatgatgtgtgaccctgttgcctttacttggtctgcatcttaccctacatatatataatctataatagagacactattggtatctcttgtgcctacaatggagtcagtcccttgtaggtttcccaagaacccagagattccctttggtttggtttgtagaagaaagggcaagactacaggcctttgtccctctctcccggcagcacaggcgagtggggcaataggaagtataaacatgaacgtgaacttggtcattcacccagagcagcccaacagccctggggccatgagaatccaagcaggctcctacatcccatttcctcatctggtgatgagacatgctcttccttagcacgtcaatgagagactgtggctttgtgcccacattggctcctgacaggggattgacagccaagaccacactgcctcccacaccagcctcctctaacacccatgttctgttgctgccttgacttccgtgccctgctcttactcctgcctattcaagcttttctgcctgcagcactcctgctgtctccctgcccgcaggagtcccggtgtctgctttgtgtttttcccgtcggaaccccagcctcactagcacgtggtaacttgtcagggcccctggctctgacctgaggattccctgagcctggcttggaacagaacaggtgtctcttaacattggttgagtgaatacatgtgcttgaatagaagcatgaattcatctttcatggcttccctcctcccatggtgttccactgtggctccttctttggtccctgaggacctaagttctttctcctctgacctctgccccctcctctttcttctcccaggtgtactgtacctctcctcctctcccattgacacctccctctacccgtggggcctgtaaaccccagaattgccccactcacccccacatgttcatgacatgcaaaggtcatggtgacttcaaatgacgttcgggtgctgttgaggcccagggtagaggtttctttgtggtcccttgattccagttctccctctgcttgatatatttgaggctgctctgaatagactggttggctctgtcctaacccaagctcccttgagtcccaatttccgggaggcagaagccaggataaatagacagctgccactactatgggccccggggggttctgcagatctagctgcagtcgcaagagcaattgccatgcagttgacatttgggagtaacatatgtctaccctctgctagtctggcattcactcctcgccaacttagagaacttagaagcccacagaggtcaggcaggatgatagatggactgtccctgatattcgttctgtctgtgatttctgcttctctatatcccagtggtagggatcaaaatccacgtgtctggtaggtgagctttaaactgccaacccctaaattttaagtgttgtcagtcaattcacttaaaatgctatcatcaccaaaaccaaagtgtgcctaggctggttttgatccatttggtacttgccctttcagtggtcatggggaatattctttgtagatttggccataggcagtcttctgtaactgcttcctgccacatcctagcttctctgttccagcaaactgctctctctctctctctctctctctctctctctctctctctctctctctcacacacacacacacacacacacacacacacacaggtgtgctaatgagtccacgtgtctatgtggaagctagaggacaacctcaggtggcatccttaggaaccccatctgcctcctttaagacagtttccccttggcttggtgtccaccagttagacttagttggttggcctgaaaagacccagaaatctccctttctctgcctcccaagtactgagattacaagtgtgtgccgccatgcccagcctccttacatggggtctcaggggttgaatttaggtccttaagcttgcaagacaaagtccttactaactgagccatatcctcagcccaatgttttgttcttcttggagtacttttgtctgctttagaaaattgtctccggtcaacagcagggagcgttgggctgggcagctgggaagtgagcacacaggcatgtaagggtgatgcatctgccatcgtagtagaggtgagctagggtctttagcctcttggcccttggcctattgctctaacctgcccctgggacaagaggcaaactgcaaagacaccctgtctccaagatccccagactcaccctcccatcctgtgcttcacagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcctgacccctgacaggggtaacaccctgcaaagggacccccgagaccctgaacccatggaacttcatgacttttgctggatccatttcccttagtggcttccagagccccagttgcaggtcaagtgagggctgagacagctagagtggtcaaaaactgccatggtgttttatgggggcagtcccaggaagttgttgctcttccatgacccctctggatctcctgggctcttgcctgattcttgcttctgagaggccccagtattttttccttctaaggagctaacatcctcttccatgaatagcacagctcttcagcctgaatgctgacactgcagggcggttccagcaagtaggagcaagtggtggcctggtagggcacagaggcccttcaggttagtgctaaactcttaggaagtaccctctccaagcccaccgaaattcttttgatgcaagaatcagaggccccatcaggcagagttgctctgttataggatggtagggctgtaactcagtggtccagtgtgcttttagcatgccctgggtttgatcctcagcaacacatgcaaaacgtaagtagacagcagacagcagacagcacagccagccccctgtgtggtttgcagcctctgcctttgacttttactctggtgggcacacagagggctggagctcctcctcctgaccttctaatgagcccttccaaggccacgccttccttcagggaatctcagggactgtagagttcccaggcccctgcagccacctgtctcttcctacctcagcctggagcactccctctaactccccaacggcttggtactgtacttgctgtgaccccaagtgcatgtccgggttaggcactgtgagttggaacagctgatgacatcggttgaaaggcccacccggaaacagctgaagccagctcttttgccaaaggattcatgccggttttctaatcaacctgctcccctagcatgcctggaaggaaagggttcaggagactcctcaagaagcaagttcagtctcaggtgcttggatgccatgctcaccgattccactggatatgaacttggcagaggagcctagttgttgccatggagacttaaagagctcagcactctggaatcaagatactggacacttggggccgacttgttaaggctctgcagcatcagactgtagaggggaaggaacacgtctgccccctggtggcccgtcctgggatgacctcgggcctcctaggcaacaaaagaatgaattggaaaggactgttcctgggtgtggcctcagctcctgtgcttgtgtggatccctaaagggtgtgctaaggagcaattgcactgtgtgctggacagaattcctgcttataaatgctttttgttgttgttttgtacactgagccctggctgagccaccccaccccacctcccatcccacctttacagccactcttgcagagaacctggctgtctcccacttgtagcctgtggatgctgaggaaacacccagccaagtagactccaggcttgcccctatctcctgctctgagtctggcctcctcattgtgttgtgggaaggagacgggttctgtcatctcggaagcccacaccgtggatgtgaacaatggctgtactagcttagaccagcttagggctctgcaatcagaggagggggagcagggaacaatttgagtgctgacctataacacattcctaaaggatgggcagtccagaatctccctccttcagtgtgtgtgtgtgtgtgtgtgtgtgtgtgtgtgtgtgtgtgtccatgtttgcatgtatgtgtgtgccagtgtgtggaggccagaggttggctttgggtgtgtttgatcactctcagttactgaggcagggctctcatctgtacccagagcttgcacattttctagtctaacttgcttcagggatctctgtctgcctatggagtgctcaggttacaggcaggctgccatacctgcccgacatttacatgaatactagagatctgaattctggtcctcacacttgtatacctgcattttatccactaagacatctctccaagggctcccccttcctatttaataagttagttttgaactggcaagatggctcagtgggtaaggcagtttgcggacaaacctgatgacctgagttggatccctgaccataaggtagaagagacctgattcctgcaagttgtcctctgaccaccaccccatacatgcttctgcatatgtgcacacatcacattcttgcacacacactcacataccataaatgtaataaatttttttaaataaattgattttatcttttaatcattatttttgcttatctgtttgttcatacatggagttttgctgtatacccagtctggctgcatcctccaggcaccttgaacttggatattctcctgcctcagtttcgagagtgctgagttataggcatgtgcaaccgtgtcaggacccttggcgttacattatgttttcgaatcttatttttccatctaagactgacaaccaccttgccaaaggatgaccttgacttttgtatcctcttgcctctacttcccaagtattgggatgacaagtctgtccatcctatgatttgtgtggtgctggggcttgaacccaggggcctcgtgaatgctaggcaaacgcaatcagctgcccaaccccgaacttcacttttcacctgtgatttcttaacatggttcctgagcagacagcatgatcctgttgccttgaggagctctgctcgttgctttgacaatgctctatttactaaagaatacttatagagttcaagactccaaagcagcagttcccaacctgtgggtatgacctcttgggtatcccatatcagatatttacattaaacttataacagaagcaaaattacagttatggggtagcaacaaaataatttttttttgaggcagggtttatctgtgtagctcactttgtacagactaggctggccttgaacttaaagatctgcctgcctctgccttctgaatgctgggattaaatgtgtgtgccaacatgcccagtgcagtgaaataattttattgttgggtcaccaaaacatgagaaagagtattaaaaggtcatagcgttaggaaggttgagaaccactgccccaaaagatagtagaaataggtaatagcattttaaaaaaagattaatttctttttccgtgtgtgtgtgtgtgtgtgtgtgtgtgtgtgtgtgtgtgtgtgtgtgtgtgtgtgaagggtgctgaattccctaaatctgggattacagacaattgtgccaaatgggtattgggaaccaaagttgggttttctgaaagagcagcaagtgcccttaactgctgagccctctctctagctataataaacttttttttaaaaaaattaaaaaaatttagcactc

SEQ ID NO: 16 - DNA, 4913 bp, Mus musculus
  source 1..4913: mol_type = genomic DNA
agtcaccagctagagcgcagctgaggcactagagctccaggcacaagggcgggagccaccgctgcccctatggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattggtctgattgttggagtgacatcactgggtctgctgatgttaggactggtgaactgcatcatcctggtgcagaggaaaaagaagccctcctgcctacaaagagatgccaaggtgcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcctgacccctgacaggggtaacaccctgcaaagggacccccgagaccctgaacccatggaacttcatgacttttgctggatccatttcccttagtggcttccagagccccagttgcaggtcaagtgagggctgagacagctagagtggtcaaaaactgccatggtgttttatgggggcagtcccaggaagttgttgctcttccatgacccctctggatctcctgggctcttgcctgattcttgcttctgagaggccccagtattttttccttctaaggagctaacatcctcttccatgaatagcacagctcttcagcctgaatgctgacactgcagggcggttccagcaagtaggagcaagtggtggcctggtagggcacagaggcccttcaggttagtgctaaactcttaggaagtaccctctccaagcccaccgaaattcttttgatgcaagaatcagaggccccatcaggcagagttgctctgttataggatggtagggctgtaactcagtggtccagtgtgcttttagcatgccctgggtttgatcctcagcaacacatgcaaaacgtaagtagacagcagacagcagacagcacagccagccccctgtgtggtttgcagcctctgcctttgacttttactctggtgggcacacagagggctggagctcctcctcctgaccttctaatgagcccttccaaggccacgccttccttcagggaatctcagggactgtagagttcccaggcccctgcagccacctgtctcttcctacctcagcctggagcactccctctaactccccaacggcttggtactgtacttgctgtgaccccaagtgcatgtccgggttaggcactgtgagttggaacagctgatgacatcggttgaaaggcccacccggaaacagctgaagccagctcttttgccaaaggattcatgccggttttctaatcaacctgctcccctagcatgcctggaaggaaagggttcaggagactcctcaagaagcaagttcagtctcaggtgcttggatgccatgctcaccgattccactggatatgaacttggcagaggagcctagttgttgccatggagacttaaagagctcagcactctggaatcaagatactggacacttggggccgacttgttaaggctctgcagcatcagactgtagaggggaaggaacacgtctgccccctggtggcccgtcctgggatgacctcgggcctcctaggcaacaaaagaatgaattggaaaggactgttcctgggtgtggcctcagctcctgtgcttgtgtggatccctaaagggtgtgctaaggagcaattgcactgtgtgctggacagaattcctgcttataaatgctttttgttgttgttttgtacactgagccctggctgagccaccccaccccacctcccatcccacctttacagccactcttgcagagaacctggctgtctcccacttgtagcctgtggatgctgaggaaacacccagccaagtagactccaggcttgcccctatctcctgctctgagtctggcctcctcattgtgttgtgggaaggagacgggttctgtcatctcggaagcccacaccgtggatgtgaacaatggctgtactagcttagaccagcttagggctctgcaatcagaggagggggagcagggaacaatttgagtgctgacctataacacattcctaaaggatgggcagtccagaatctccctccttcagtgtgtgtgtgtgtgtgtgtgtgtgtgtgtgtgtgtgtgtgtccatgtttgcatgtatgtgtgtgccagtgtgtggaggccagaggttggctttgggtgtgtttgatcactctcagttactgaggcagggctctcatctgtacccagagcttgcacattttctagtctaacttgcttcagggatctctgtctgcctatggagtgctcaggttacaggcaggctgccatacctgcccgacatttacatgaatactagagatctgaattctggtcctcacacttgtatacctgcattttatccactaagacatctctccaagggctcccccttcctatttaataagttagttttgaactggcaagatggctcagtgggtaaggcagtttgcggacaaacctgatgacctgagttggatccctgaccataaggtagaagagacctgattcctgcaagttgtcctctgaccaccaccccatacatgcttctgcatatgtgcacacatcacattcttgcacacacactcacataccataaatgtaataaatttttttaaataaattgattttatcttttaatcattatttttgcttatctgtttgttcatacatggagttttgctgtatacccagtctggctgcatcctccaggcaccttgaacttggatattctcctgcctcagtttcgagagtgctgagttataggcatgtgcaaccgtgtcaggacccttggcgttacattatgttttcgaatcttatttttccatctaagactgacaaccaccttgccaaaggatgaccttgacttttgtatcctcttgcctctacttcccaagtattgggatgacaagtctgtccatcctatgatttgtgtggtgctggggcttgaacccaggggcctcgtgaatgctaggcaaacgcaatcagctgcccaaccccgaacttcacttttcacctgtgatttcttaacatggttcctgagcagacagcatgatcctgttgccttgaggagctctgctcgttgctttgacaatgctctatttactaaagaatacttatagagttcaagactccaaagcagcagttcccaacctgtgggtatgacctcttgggtatcccatatcagatatttacattaaacttataacagaagcaaaattacagttatggggtagcaacaaaataatttttttttgaggcagggtttatctgtgtagctcactttgtacagactaggctggccttgaacttaaagatctgcctgcctctgccttctgaatgctgggattaaatgtgtgtgccaacatgcccagtgcagtgaaataattttattgttgggtcaccaaaacatgagaaagagtattaaaaggtcatagcgttaggaaggttgagaaccactgccccaaaagatagtagaaataggtaatagcattttaaaaaaagattaatttctttttccgtgtgtgtgtgtgtgtgtgtgtgtgtgtgtgtgtgtgtgtgtgtgtgtgtgtgtgaagggtgctgaattccctaaatctgggattacagacaattgtgccaaatgggtattgggaaccaaagttgggttttctgaaagagcagcaagtgcccttaactgctgagccctctctctagctataataaacttttttttaaaaaaattaaaaaaatttagcactc

SEQ ID NO: 17 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 18 - DNA, 20 bp, synthetic construct
  misc_feature 1..20: note = gRNA sequence
  source 1..20: mol_type = other DNA
cggacagtcactcaccaagt

SEQ ID NO: 19 - DNA, 22 bp, synthetic construct
  misc_feature 1..22: note = Single strand DNA oligonucleotide
  source 1..22: mol_type = other DNA
gaacaattccatctgctgcacc

SEQ ID NO: 20 - DNA, 21 bp, synthetic construct
  misc_feature 1..21: note = Single strand DNA oligonucleotide
  source 1..21: mol_type = other DNA
ttcgaaatcctgacctcctgg

SEQ ID NO: 21 - DNA, 20 bp, synthetic construct
  misc_feature 1..20: note = gRNA sequence
  source 1..20: mol_type = other DNA
gacactgcctgaggtaattc

SEQ ID NO: 22 - DNA, 22 bp, synthetic construct
  misc_feature 1..22: note = Single strand DNA oligonucleotide
  source 1..22: mol_type = other DNA
tgagacagggtttctctatacc

SEQ ID NO: 23 - DNA, 18 bp, synthetic construct
  misc_feature 1..18: note = Single strand DNA oligonucleotide
  source 1..18: mol_type = other DNA
catggcattcgtctttgt

SEQ ID NO: 24 - DNA, 20 bp, synthetic construct
  misc_feature 1..20: note = gRNA sequence
  source 1..20: mol_type = other DNA
cggcttcccagaattacctc

SEQ ID NO: 25 - DNA, 20 bp, synthetic construct
  misc_feature 1..20: note = Single strand DNA oligonucleotide
  source 1..20: mol_type = other DNA
agtgagagcagagaattgtc

SEQ ID NO: 26 - DNA, 20 bp, synthetic construct
  misc_feature 1..20: note = Single strand DNA oligonucleotide
  source 1..20: mol_type = other DNA
gagcttgcatgtgcacgcat

SEQ ID NO: 27 - DNA, 20 bp, synthetic construct
  misc_feature 1..20: note = gRNA sequence
  source 1..20: mol_type = other DNA
atatacccctcaggggttat

SEQ ID NO: 28 - DNA, 22 bp, synthetic construct
  misc_feature 1..22: note = Single strand DNA oligonucleotide
  source 1..22: mol_type = other DNA
tctcacactccctgcagtccgt

SEQ ID NO: 29 - DNA, 22 bp, synthetic construct
  misc_feature 1..22: note = Single strand DNA oligonucleotide
  source 1..22: mol_type = other DNA
cagattgtatggccccaactgt

SEQ ID NO: 30 - DNA, 20 bp, synthetic construct
  misc_feature 1..20: note = gRNA sequence
  source 1..20: mol_type = other DNA
attggactggtccctcacct

SEQ ID NO: 31 - DNA, 21 bp, synthetic construct
  misc_feature 1..21: note = Single strand DNA oligonucleotide
  source 1..21: mol_type = other DNA
ttctgaagcggtgaaggagcc

SEQ ID NO: 32 - DNA, 22 bp, synthetic construct
  misc_feature 1..22: note = Single strand DNA oligonucleotide
  source 1..22: mol_type = other DNA
cagattgtatggccccaactgt

SEQ ID NO: 33 - DNA, 20 bp, synthetic construct
  misc_feature 1..20: note = gRNA sequence
  source 1..20: mol_type = other DNA
tacttgtacaatgactgtcc

SEQ ID NO: 34 - DNA, 21 bp, synthetic construct
  misc_feature 1..21: note = Single strand DNA oligonucleotide
  source 1..21: mol_type = other DNA
tgccggtactggttcttcctg

SEQ ID NO: 35 - DNA, 22 bp, synthetic construct
  misc_feature 1..22: note = Single strand DNA oligonucleotide
  source 1..22: mol_type = other DNA
gggtgctgcttctttctctgct

SEQ ID NO: 36 - DNA, 20 bp, synthetic construct
  misc_feature 1..20: note = gRNA sequence
  source 1..20: mol_type = other DNA
taatgtatcgctaccaacgg

SEQ ID NO: 37 - DNA, 17 bp, synthetic construct
  misc_feature 1..17: note = Single strand DNA oligonucleotide
  source 1..17: mol_type = other DNA
accacagtgctgttgcc

SEQ ID NO: 38 - DNA, 18 bp, synthetic construct
  misc_feature 1..18: note = Single strand DNA oligonucleotide
  source 1..18: mol_type = other DNA
tcacctccctccacacat

SEQ ID NO: 39 - DNA, 20 bp, synthetic construct
  misc_feature 1..20: note = gRNA sequence
  source 1..20: mol_type = other DNA
cactccaataatgccggtac

SEQ ID NO: 40 - DNA, 21 bp, synthetic construct
  misc_feature 1..21: note = Single strand DNA oligonucleotide
  source 1..21: mol_type = other DNA
atctcttcttgcacagtggac

SEQ ID NO: 41 - DNA, 20 bp, synthetic construct
  misc_feature 1..20: note = Single strand DNA oligonucleotide
  source 1..20: mol_type = other DNA
acggtgttctgtttctcctg

SEQ ID NO: 42 - DNA, 20 bp, synthetic construct
  misc_feature 1..20: note = gRNA sequence
  source 1..20: mol_type = other DNA
agaggtgcacggtcccattg

SEQ ID NO: 43 - DNA, 21 bp, synthetic construct
  misc_feature 1..21: note = Single strand DNA oligonucleotide
  source 1..21: mol_type = other DNA
gaagaaccagtaccggcatta

SEQ ID NO: 44 - DNA, 21 bp, synthetic construct
  misc_feature 1..21: note = Single strand DNA oligonucleotide
  source 1..21: mol_type = other DNA
gttgtcagacccacagaatac

SEQ ID NO: 45 - DNA, 20 bp, synthetic construct
  misc_feature 1..20: note = gRNA sequence
  source 1..20: mol_type = other DNA
ttggactggtccctcaccta

SEQ ID NO: 46 - DNA, 22 bp, synthetic construct
  misc_feature 1..22: note = Single strand DNA oligonucleotide
  source 1..22: mol_type = other DNA
tctcttgatggtgtctcctcta

SEQ ID NO: 47 - DNA, 22 bp, synthetic construct
  misc_feature 1..22: note = Single strand DNA oligonucleotide
  source 1..22: mol_type = other DNA
actggaagaagcagagaaagaa

(19) United States
(12) Patent Application Publication
(10) Pub. No.: US 2026/0130945 A1
(43) Pub. Date: May 14, 2026

(54) METHODS OF TREATING AN ISCHEMIC DISEASE

(71) Applicant: Dalia ELANI, Petach-Tikva (IL)

(72) Inventor: Dalia ELANI, Petach-Tikva (IL)

(21) Appl. No.: 19/260,774

(22) Filed: Jul. 7, 2025

Related U.S. Application Data
(63) Continuation of application No. 18/200,595, filed on May 23, 2023, now abandoned.
Application No. 18/200,595 is a continuation of application No. 16/758,457, now Pat. No. 11,701,391.
(60) Provisional application No. 62/576,094, filed on Oct. 24, 2017.

(51) Int. Cl. A61K 35/28; A01N 1/162; A61K 40/10; A61K 40/40; A61P 9/10; C12N 5/077; C12N 9/22; C12N 15/113

(52) U.S. Cl. CPC A61K 35/28; A01N 1/162; A61K 40/10; A61K 40/40; A61P 9/10; C12N 5/0669; C12N 9/22; C12N 15/113; A61K 2239/31; A61K 2239/38; C12N 2310/20; C12N 2510/00; C12N 2800/80

(57) ABSTRACT

Methods of treating an ischemic disease in a subject are provided. Accordingly there is provided a method comprising administering to the subject a therapeutically effective amount of cells with reduced level of expression and/or activity of TNFR1, thereby treating the ischemic disease in the subject. Also provided is a method comprising treating with TNFalpha cells with reduced expression and/or activity of TNFR1 and administering to the subject a therapeutically effective amount of said cells, thereby treating the ischemic disease in the subject.

### RELATED APPLICATIONS

This application is a continuation of U.S. patent application Ser. No. 18/200,595, filed on May 23, 2023, which is a continuation of U.S. patent application Ser. No. 16/758,457, filed on Apr. 23, 2020, now U.S. Pat. No. 11,701,391, which is a National Phase of PCT Patent Application No. PCT/IL2018/051139 having International Filing Date of Oct. 24, 2018, which claims the benefit of priority under 35 USC § 119(e) of U.S. Provisional Patent Application No. 62/576,094 filed on Oct. 24, 2017. The contents of the above applications are all incorporated by reference as if fully set forth herein in their entirety.

### SEQUENCE LISTING STATEMENT

The XML file, entitled SequenceListing.xml, created on Jul. 1, 2025, comprising 177,143 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to methods of treating an ischemic disease.

Ischemic heart disease, such as acute myocardial infarctions, congestive heart failure, arrhythmias, and sudden cardiac death, is the leading cause of morbidity and mortality in all industrialized nations. In the United States, ischemic heart disease causes nearly 20% of all deaths (≈600,000 deaths each year). Over the past half a century conventional medicine and surgery have offered many breakthroughs, resulting in a dramatic decline in mortality [Mozaffarian et al. Circulation. (2015) 131(4):e29-322.) Despite the major advances in treatment, the prognosis for patients who are admitted to hospital with heart failure remains poor, with a 5 year survival of about 50% and a 10 year survival of about 10%. The approach of using stem or precursor cells has emerged in the last decade as a regenerative strategy to address cardiac disease, with pre-clinical and clinical trials showing beneficial effects of progenitor cell therapy in acute myocardial infarction and ischemic cardiomyopathy [e.g. Choudhury et al. Eur J Heart Fail. (2017) 19(1):138-147; and Karantalis et al. Circ Res. (2015) 116(8):1413-30].

Tumor necrosis factor alpha (TNFα) is a pro-inflammatory cytokine that has been implicated in mediating or exacerbating various mammalian conditions such as myocardial infarction, heart failure, septic shock, inflammatory disease, HIV infection and tissue transplant. However, in anti-cytokine clinical trials, the use of either a soluble TNF receptor, or an anti-TNF antibody was not beneficial to patients with heart failure. In addition, treatment with soluble TNF receptors significantly exacerbated ventricular dysfunction and remodeling with enhanced interstitial fibrosis after myocardial infarction (Monden et al [34]). These findings suggest that TNFα may not be exclusively toxic but may be partially protective in cardiovascular diseases. TNFα initiates its biological effects by binding to two distinct cell surface receptors expressed in most cell types: TNFR1 (TNFα receptor type 1, with approximate mass of 55 kDa) and TNFR2 (TNF-α receptor type 2, with approximate mass of 75 kDa). The specific roles of TNFR1 and TNFR2 signaling in ischemic damage remain unclear with several studies indicating that signaling via TNFR1 is deleterious and signaling via TNFR2 is protective against ischemic damage while others suggesting no difference in the function of the two receptors in protecting the heart from ischemia (see e.g. Kishore et al. [33], Monden et al [34], Moe et al 2004 and Kurrelmeyer et al [37]).

Additional background art includes:
International Patent Application Publication Nos. WO2000000504, WO2015104322 and WO2011006914;
US Patent Application Publication Nos: US20120014879 and US20110189768;
European Patent No. EP2746396;
Chinese Patent No. CN101401930;
Bao et al. (2008) Scand. Cardiovasc. J. 42, 56-62;
Farhang et al. (2017) Tissue Eng Part A. 23(15-16):738-749;
Kelly et al. (2010) Shock. 33(6): 602-607; and
Tan et al. (2010) Shock. 34(3): 236-42.

### SUMMARY OF THE INVENTION

According to an aspect of some embodiments of the present invention there is provided a method of treating an ischemic disease in a subject in need thereof, the method comprising administering to the subject a therapeutically effective amount of mononuclear bone marrow cells (mnBMCs) comprising mesenchymal stem cells (MSCs) and lymphocytes or progenitors thereof with reduced level of expression and/or activity of TNFR1 as compared to control cells of the same origin not contacted with an agent which downregulates expression and/or activity of the TNFR1, thereby treating the ischemic disease in the subject.

According to an aspect of some embodiments of the present invention there is provided a method of treating an ischemic disease in a subject in need thereof, the method comprising administering to the subject a therapeutically effective amount of differentiated cells with reduced level of expression and/or activity of TNFR1 as compared to control cells of the same origin not contacted with an agent which downregulates expression and/or activity of the TNFR1, wherein the differentiated cells are of a same type of tissue affected in the ischemic disease, thereby treating the ischemic disease in the subject.

According to some embodiments of the invention, the method further comprising treating the cells with reduced expression and/or activity of TNFR1 with TNFα prior to the administering.

According to some embodiments of the invention, the method comprising cryopreserving the cells prior to the treating with the TNFα.

According to an aspect of some embodiments of the present invention there is provided a method of treating an ischemic disease in a subject in need thereof, the method comprising:
(i) treating with TNFα cells with reduced expression and/or activity of TNFR1 as compared to control cells of the same origin not contacted with an agent which downregulates expression and/or activity of the TNFR1, wherein the cells are selected from the group consisting of mononuclear bone marrow cells (mnBMCs), stem cells and differentiated cells of a same type of tissue affected in the ischemic disease; and
(ii) administering to the subject a therapeutically effective amount of the cells with reduced level of expression and/or activity of TNFR1 following the (i), thereby treating the ischemic disease in the subject.

According to some embodiments of the invention, the cells comprise differentiated cells of a same type of tissue affected in the ischemic disease.

According to some embodiments of the invention, the cells with reduced level of expression and/or activity of TNFR1 have the same level of expression and/or activity of TNFR2 as compared to the control stem cells.

According to some embodiments of the invention, the cells with reduced expression and/or activity of TNFR1 are genetically modified cells.

According to some embodiments of the invention, the genetically modified comprises genetically modified with a CRISPR/Cas system, a Zinc finger nuclease (ZFN), transcription-activator like effector nuclease (TALEN) or meganuclease for downregulating expression of the TNFR1.

According to some embodiments of the invention, the genetically modified comprises genetically modified with a CRISPR/Cas system for downregulating expression of the TNFR1.

According to an aspect of some embodiments of the present invention there is provided a method of downregulating expression and/or activity of TNFR1 in differentiated cells or bone marrow stem cells, the method comprising:
(i) contacting ex-vivo or in-vitro differentiated cells or bone marrow stem cells with an agent which downregulates expression and/or activity of TNFR1; and
(ii) contacting ex-vivo or in-vitro the cells with TNFα.

According to some embodiments of the invention, the (i) is effected prior to the (ii).

According to some embodiments of the invention, the agent does not downregulate expression and/or activity of TNFR2.

According to some embodiments of the invention, the agent is selected from the group consisting of CRISPR/Cas system, a Zinc finger nuclease (ZFN), transcription-activator like effector nuclease (TALEN), meganuclease, antisense and siRNA.

According to some embodiments of the invention, the agent comprises a CRISPR/Cas system.

According to some embodiments of the invention, there is provided a method of treating an ischemic disease in a subject in need thereof, the method comprising:
(i) obtaining cells according to the method of the present invention; and
(ii) administering to the subject a therapeutically effective amount of the cells, thereby treating the ischemic disease in the subject.

According to some embodiments of the invention, the differentiated cells are of a same type of tissue affected in the ischemic disease.

According to some embodiments of the invention, the method comprising cryopreserving the cells following the (i) and prior to the (ii).

According to some embodiments of the invention, the cells are non-autologous to the subject.

According to an aspect of some embodiments of the present invention there is provided a method of treating an ischemic disease in a subject in need thereof, the method comprising administering to the subject a therapeutically effective amount of a CRISPR/Cas system for downregulating expression of TNFR1, thereby treating the ischemic disease in the subject.

According to an aspect of some embodiments of the present invention there is provided a pharmaceutical composition comprising as an active ingredient differentiated cells or bone marrow stem cells genetically modified with a CRISPR/Cas system for downregulating expression of TNFR1.

According to an aspect of some embodiments of the present invention there is provided a pharmaceutical composition comprising as active ingredients cells genetically modified with a CRISPR/Cas system for downregulating expression of TNFR1 and at least 2 ng/ml TNFα.

According to some embodiments of the invention, the CRISPR/Cas system does not downregulate expression of TNFR2.

According to some embodiments of the invention, the cells comprise differentiated cells.

According to some embodiments of the invention, the differentiated cells are cardiomyocytes.

According to some embodiments of the invention, the cells comprise stem cells.

According to some embodiments of the invention, the stem cells are selected from the group consisting of embryonic stem cells (ESCs), induced pluripotent stem cells (iPS), hematopoietic stem cells (HSCs) and mesenchymal stem cells (MSCs).

According to some embodiments of the invention, the stem cells comprise hematopoietic stem cells (HSCs).

According to some embodiments of the invention, the cells are comprised in mononuclear bone marrow cells (mnBMCs).

According to some embodiments of the invention, the mnBMCs comprise lymphocytes or progenitors thereof.

According to some embodiments of the invention, the cells are obtained by density gradient centrifugation of bone marrow cells.

According to some embodiments of the invention, the method comprising obtaining the cells by density gradient centrifugation of bone marrow cells.

According to some embodiments of the invention, the cells are human cells.

According to some embodiments of the invention, the cells are cryopreserved cells.

According to some embodiments of the invention, the ischemic disease is ischemic heart disease.

According to some embodiments of the invention, the ischemic heart disease is myocardial infarction.

According to some embodiments of the invention, the ischemic heart disease is ischemic cardiomyopathy.

According to some embodiments of the invention, the subject is not treated with TNFα.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1F show representative photomicrographs of histological sections of hearts obtained from Control mice (Group 3M, see Table 2 hereinbelow) 24 hours following left anterior descending artery (LAD) occlusion and stained with Masson Trichrome (MT, FIGS. 1A, 1C, 1E) or Hematoxylin & Eosin (H&E, FIGS. 1B, 1D, 1F). FIGS. 1A-1B demonstrate a middle sized infarct volume (FIG. 1A) and cellular infiltration surrounding injected round clear cells (arrow, FIG. 1B) in mouse no. 15. FIGS. 1C-1D demonstrate middle sized infarct volume (FIG. 1C) and cellular infiltration surrounding injected round clear cells (arrow) and some adipocytes (FIG. 1D) in mouse no. 16. FIGS. 1E-1F demonstrate middle sized infarct volume (FIG. 1E) and marked cellular infiltration in and few injected cells (arrow, FIG. 1F) in mouse no. 18.

FIGS. 2A-2F show representative photomicrographs of histological sections of hearts obtained from Group 2M mice (see Table 2 hereinbelow) 24 hours following LAD occlusion and stained with MT (FIGS. 2A, 2C, 2E) or H&E (FIGS. 2B, 2D, 2F). FIGS. 2A-2B demonstrate a small infarct volume (FIG. 2A) and mild cellular infiltration (FIG. 2B) in mouse no. 7. FIGS. 2C-2D demonstrate middle sized infarct volume (FIG. 2C) and focal extensive infiltration with some cellular debris of the injected cells (arrow, FIG. 2D) in mouse no. 9. FIGS. 2E-2F a transmural infarct with still intact left ventricle wall (FIG. 2E) and marked cellular infiltration in the inner part of the ventricle wall (FIG. 2F) in mouse no. 10.

FIGS. 3A-3F show representative photomicrographs of histological sections of hearts obtained from Group 1M mice (see Table 2 hereinbelow) 24 hours following LAD occlusion and stained with MT (FIGS. 3A, 3C, 3E) or H&E (FIGS. 3B, 3D, 3F). FIGS. 3A-3B demonstrate a transmural infarct with a marked reduced myocardium tissue (FIG. 3A) and minimal cellular infiltration (FIG. 3B) in mouse no. 3. FIGS. 3C-D, demonstrate a relative small infarct volume in the wall of the left ventricle (FIG. 3C) and loss of tissue, inflammatory reaction and some fibrin material in the epicardium (arrow, FIG. 3D) in mouse no. 5. FIGS. 3E-3F demonstrate a transmural infarct with a marked reduced myocardium tissue (FIG. 3E) and mild infiltration of inflammatory cells (FIG. 3F) in mouse no. 3.

FIGS. 4A-4D show representative photomicrographs of histological sections of hearts obtained from Control mice (Group 3M, see Table 2 hereinbelow) 4 days following LAD occlusion and stained with MT (FIGS. 4A, 4C) or H&E (FIGS. 4B, 4D). FIGS. 4A-4B demonstrate a very large infarct volume (FIG. 4A) and severe inflammation and necrosis (N) in the affected myocardium (FIG. 4B) in mouse no. 13. FIGS. 4C-4D demonstrate a very large infarct volume (FIG. 4C) and severe inflammation and necrosis (N) in the affected myocardium (FIG. 4D) in mouse no. 14.

FIGS. 5A-5D show representative photomicrographs of histological sections of hearts obtained from Group 2M mice (see Table 2 hereinbelow) 4 days following LAD occlusion and stained with MT (FIGS. 5A, 5C) or H&E (FIGS. 5B, 5D). FIGS. 5A-5B demonstrate a very large infarct volume (FIG. 5A) and severe inflammation and necrosis in the affected myocardium (FIG. 5B) in mouse no. 11. FIGS. 5C-5D demonstrate a very large infarct volume (FIG. 5C) and severe inflammation and necrosis (N) in the affected myocardium (FIG. 5D) in mouse no. 12.

FIGS. 6A-6D show representative photomicrographs of histological sections of hearts obtained from Group 1M mice (see Table 2 hereinbelow) 4 days following LAD occlusion and stained with MT (FIGS. 6A, 6C) or H&E (FIGS. 6B, 6D). FIGS. 6A-6B demonstrate a very large infarct volume (FIG. 6A) and severe inflammation and necrosis in the affected myocardium (FIG. 6B) in mouse no. 1. FIGS. 6C-6D demonstrate a very large infarct volume (FIG. 6C) and severe inflammation and necrosis in the affected myocardium (FIG. 6D) in mouse no. 2.

FIGS. 7A-7F show representative photomicrographs of histological sections of hearts obtained from Control mice (Group 3M), Group 2M mice, and Group 1M mice (see Table 2 hereinbelow) 24 hours following LAD occlusion and stained with TUNEL. FIG. 7A demonstrate a mild to moderate TUNEL reaction in the infarct lesion in mouse no. 6 (Group 1M). FIGS. 7B-7D demonstrate high TUNEL reaction in the infarct lesion in mice no. 7 (FIG. 7B), 9 (FIG. 7C) and 10 (FIG. 7D) (Group 2M). FIGS. 7E-7F demonstrate moderate to high TUNEL reaction in the infarct lesion in mice no. 15 (FIG. 7E) and 16 (FIG. 7F) (Control mice, Group 3M).

FIGS. 8A-8F show representative photomicrographs of histological sections of hearts obtained from Control mice (Group 3M), Group 2M mice, and Group 1M mice (see Table 2 hereinbelow) 4 days following LAD occlusion and stained with TUNEL. FIGS. 8A-8B demonstrate high TUNEL reaction in the infarct lesion in mouse no. 1 and mild to moderate TUNEL reaction in the infarct lesion in mouse no. 2 (Group 1M). FIGS. 8C-8D demonstrates mild TUNEL reaction in the infarct lesion in mice no. 11 and 12 (Group 2M). FIGS. 8E-8F demonstrate high TUNEL reaction in the infarct lesion in mouse no. 13 and moderate TUNEL reaction in the infarct lesion in mouse no. 14 (Control mice, Group 3M).

FIGS. 9A-9B are bar graphs demonstrating that transplantation of mono-nuclear bone marrow cells (mnBMCs) transfected with TNFR1 CRISPR reduced infract size in the LAD occlusion mouse model compared to control mice transplanted with non-transfected mnBMCs, as determined by histological evaluation 24 hours (FIG. 9A) and 4 days (FIG. 9B) following LAD occlusion.

FIG. 10 is a bar graph demonstrating that transplantation of mnBMCs transfected with TNFR1 CRISPR had no toxic effect (such as severe muscle breakdown, acute kidney injury or autoimmune myositis) in the LAD occlusion mouse model compared to control mice transplanted with non-transfected mnBMCs, as determined by CK levels 4 days following LAD occlusion.

### DESCRIPTION OF SPECIFIC EMBODIMENTS OF THE INVENTION

The present invention, in some embodiments thereof, relates to methods of treating an ischemic disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Ischemic heart disease is the leading cause of morbidity and mortality in all industrialized nations. Over the past half a century conventional medicine and surgery have offered many breakthroughs, resulting in a dramatic decline in mortality; however, despite the major advances in treatment, the prognosis for patients who are admitted to hospital with heart failure remains poor. The approach of using stem or precursor cells has emerged in the last decade as a regenerative strategy to address cardiac disease, with pre-clinical and clinical trials showing beneficial effects of progenitor cell therapy in acute myocardial infarction and ischemic cardiomyopathy.

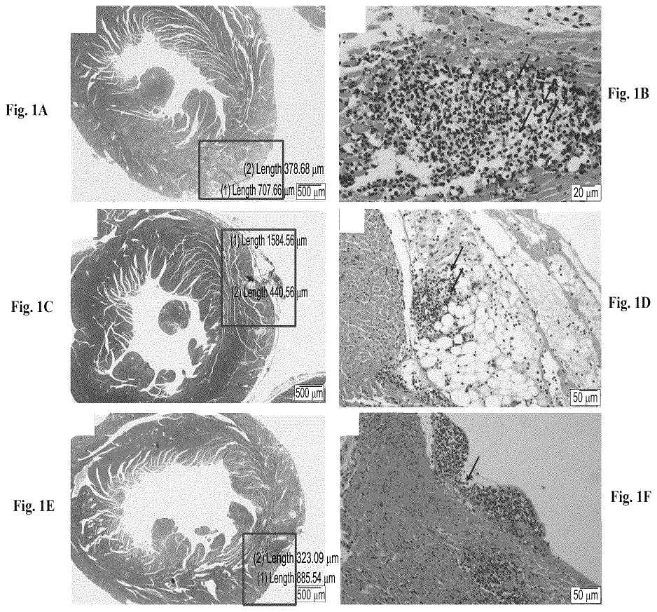

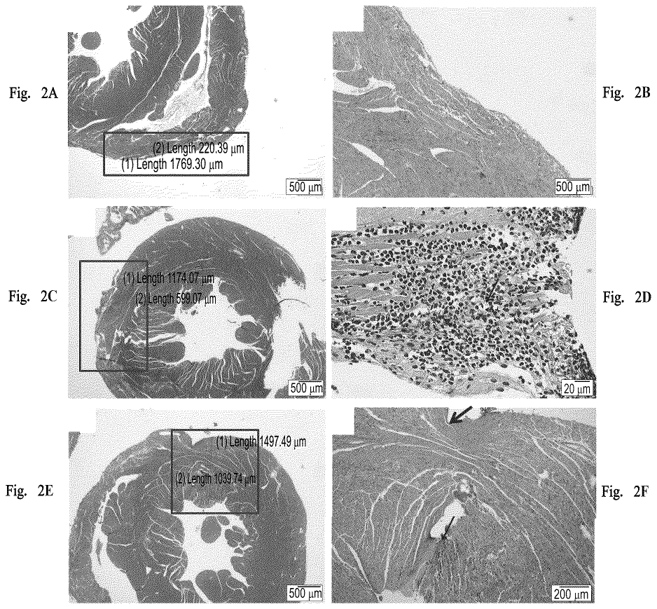

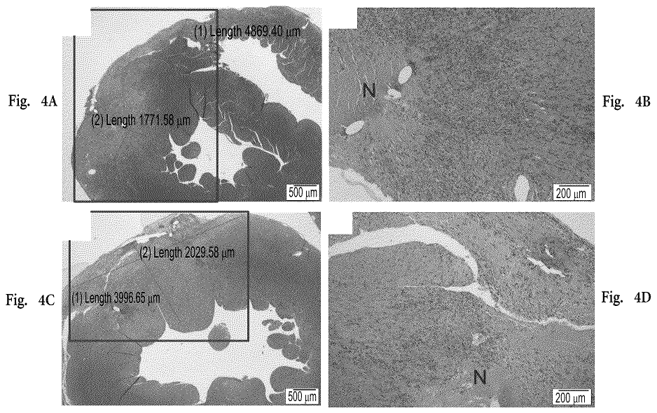

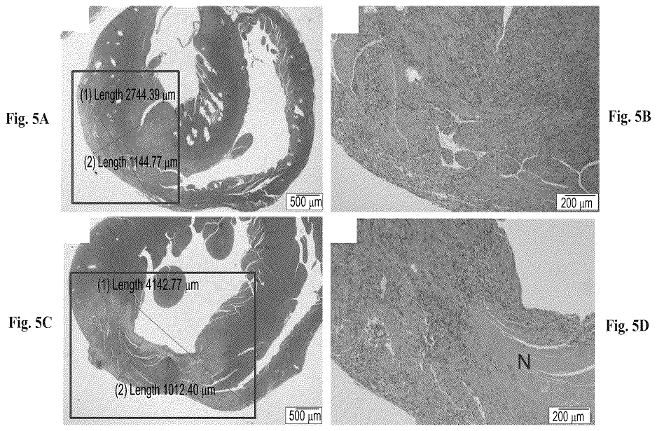

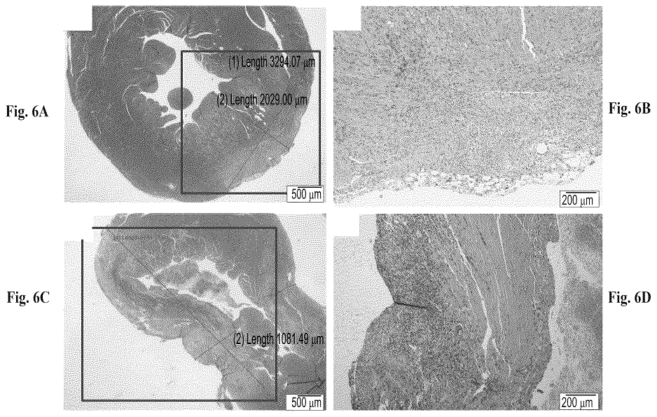

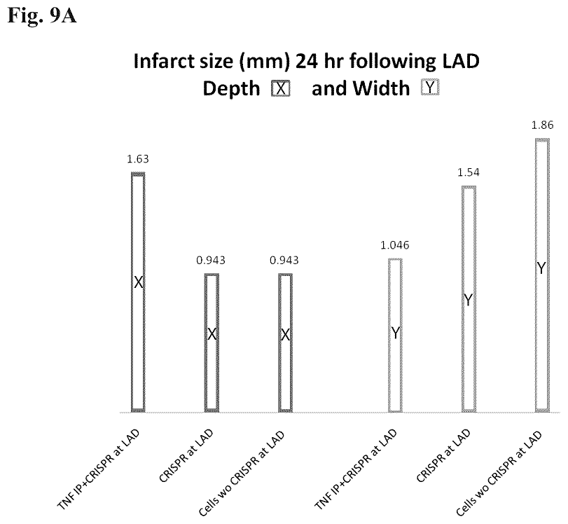

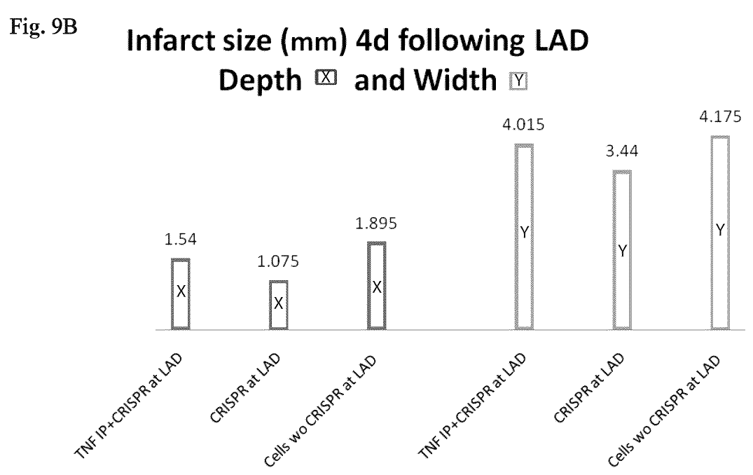

Whilst reducing the present invention to practice, the present inventors have now uncovered in a left anterior descending artery (LAD) mouse model that transplantation of mononuclear MB cells (mnBMCs) transfected with TNFR1 CRISPR/Cas system and treated with TNFα provides a cardio-protective effect from ischemic damage, as demonstrated by reduced infarct depth and width (Examples section and FIGS. 1A-10).

Consequently, some embodiments of the present teachings suggest that therapeutic cells with reduced level of expression and/or activity of TNFR1 can be used for treating an ischemic disease.

Thus, according to a first aspect of the present invention, there is provided a method of treating an ischemic disease in a subject in need thereof, the method comprising administering to the subject a therapeutically effective amount of mononuclear bone marrow cells (mnBMCs) comprising mesenchymal stem cells (MSCs) and lymphocytes or progenitors thereof with reduced level of expression and/or activity of TNFR1 as compared to control cells of the same origin not contacted with an agent which downregulates expression and/or activity of said TNFR1, thereby treating the ischemic disease in the subject.

According to another aspect of the present invention, there is provided a method of treating an ischemic disease in a subject in need thereof, the method comprising administering to the subject a therapeutically effective amount of differentiated cells with reduced level of expression and/or activity of TNFR1 as compared to control cells of the same origin not contacted with an agent which downregulates expression and/or activity of said TNFR1, wherein said differentiated cells are of a same type of tissue affected in said ischemic disease, thereby treating the ischemic disease in the subject.

According to specific embodiments, the method further comprising treating said cells with reduced expression and/or activity of TNFR1 with TNFα prior to said administering.

According to another aspect of the present invention, there is provided a method of treating an ischemic disease in a subject in need thereof, the method comprising:
(i) treating with TNFα cells with reduced expression and/or activity of TNFR1 as compared to control cells of the same origin not contacted with an agent which downregulates expression and/or activity of said TNFR1, wherein said cells are selected from the group consisting of mononuclear bone marrow cells (mnBMCs), stem cells and differentiated cells of a same type of tissue affected in said ischemic disease; and
(ii) administering to the subject a therapeutically effective amount of said cells with reduced level of expression and/or activity of TNFR1 following said (i), thereby treating the ischemic disease in the subject.

The term “treating” or “treatment” refers to inhibiting, preventing or arresting the development of a pathology (disease, disorder or medical condition e.g. ischemic disease e.g. ischemic heart disease) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “ischemic disease” refers to a disease characterized by reduced blood (and hence oxygen) supply to the diseased tissue/organ. According to specific embodiments, the ischemic disease is an acute ischemic disease. According to other specific embodiments, the ischemic disease is a chronic ischemic disease. Non-limiting Examples of ischemic diseases include trauma, ischemic cerebrovascular disorder (such as apoplexy or cerebral infarction), ischemic renal disease, ischemic pulmonary disease, infection-related ischemic disease, ischemic disease of limbs, and ischemic heart disease.

According to specific embodiments, the ischemic disease is not a rejection reaction following transplantation (e.g. GVHD).

According to specific embodiments, the ischemic disease is ischemic heart disease.

As used herein the term “ischemic heart disease” refers to a disease in which heart muscle is damaged or works inefficiently because of reduced blood (and hence oxygen) supply to the heart. Non-limiting Examples of ischemic heart diseases include ischemic cardiomyopathy, myocardial infarction or ischemic heart failure and chronic ischemic heart disease.

According to specific embodiments, the ischemic disease is myocardial infarction (also known as heart attack).

According to specific embodiments, the myocardial infarction is ST-segment elevation MI (STEMI) myocardial infarction (i.e. the coronary artery is completely blocked, thus virtually all of the heart muscle supplied by the affected artery becomes infarcted), as determined by ECG.

According to other specific embodiments, the myocardial infarction is non-ST-segment elevation MI (NSTEMI) myocardial infarction (i.e. the artery is only partially occluded thus only a portion of the heart muscle supplied by the artery becomes infarcted), as determined by ECG. According to specific embodiments, the ischemic disease is ischemic cardiomyopathy.

As used herein, the term “subject” includes mammals, e.g., human beings at any age and of any gender who suffer from the pathology (medical condition). According to specific embodiments, this term encompasses individuals who are at risk to develop the pathology.

According to specific embodiments, the subject is not treated with TNFα.

“TNFα”, a cytokine also known as Tumor necrosis factor alpha. TNFα can bind TNFR1 and TNFR2. According to specific embodiments, the TNFα protein refers to the human protein, such as provided in the following GenBank Number NP_000585 (SEQ ID NO: 1).

The term also encompasses functional homologues (naturally occurring or synthetically/recombinantly produced), orthologs (from other species) which exhibit the desired activity (i.e., binding TNFR1, binding TNFR2, activation of NFκB, activation of the MAPK pathway). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 1 or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

Sequence identity or homology can be determined using any protein or nucleic acid sequence alignment algorithm such as Blast, ClustalW, and MUSCLE.

The functional homologs also refer to functional portions of TNFα which maintain the activity of the full length protein (i.e. binding TNFR1, binding TNFR2, activation of NFκB, activation of the MAPK pathway).

According to specific embodiments, the TNFα is a recombinant TNFα, which can be prepared by standard recombinant expression methods or purchased commercially.

TNFα can be commercially obtained from e.g. R&D Systems.

According to specific embodiments, the cells are treated (or contacted) with TNFα.

According to specific embodiments, the effect of treatment with TNFα is additive.

According to other specific embodiments, the effect of treatment with TNFα is synergistic.

According to specific embodiments, treatment (or contacting) with TNFα is effected at a concentration of at least 2 ng/ml, at least 5 ng/ml, at least 10 ng/ml, at least 20 ng/ml, at least 30 ng/ml, at least 40 ng/ml, at least 50 ng/ml or at least 60 ng/ml, each possibility represents a separate embodiment of the present invention.

According to specific embodiments, treatment (or contacting) with TNFα is effected at a concentration of 2-200 ng/ml or 20-200 ng/ml.

According to specific embodiments, treatment (or contacting) with TNFα is effected 1-72 hours, 1-48, 1-24, 1-10 or 1-5 hours prior to the administering, each possibility represents a separate embodiment of the present invention.

According to specific embodiments, treatment (or contacting) with TNFα comprises a single TNFα treatment.

According to other specific embodiments, treatment (or contacting) with TNFα comprises a plurality of TNFα treatments.

Several types of cells can be used and/or obtained according to specific embodiments of the present invention.

According to specific embodiments, the cells comprise differentiated cells.

Non-limiting examples of differentiated cells that can be used with some embodiments of the present invention include differentiated cells derived from heart, kidney, liver, lung and brain.

According to specific embodiments, the differentiated cells comprise differentiated cells of a same type of tissue affected in the ischemic disease.

Methods of obtaining such differentiated cells are well known in the art. For example a cell suspension may be obtained by any mechanical or chemical (e.g. enzymatic) means. Several methods exist for dissociating cell clusters to form cell suspensions (e.g. single cell suspension) from primary tissues, attached cells in culture, and aggregates, e.g., physical forces (mechanical dissociation such as cell scraper, trituration through a narrow bore pipette, fine needle aspiration, vortex disaggregation and forced filtration through a fine nylon or stainless steel mesh), enzymes (enzymatic dissociation such as trypsin, collagenase, Acutase and the like) or a combination of both. Thus, for example, enzymatic digestion of tissue/organ into isolate cells can be performed by subjecting the tissue to an enzyme such as type IV Collagenase (Worthington biochemical corporation, Lakewood, NJ, USA) and/or Dispase (Invitrogen Corporation products, Grand Island NY, USA). For example, the tissue may be enzyme digested by finely mincing tissue with a razor blade in the presence of e.g. collagenase, dispase and CaCl2 at 37° C. for about 1 hour. The method may further comprise removal of nonspecific debris from the resultant cell suspension by, for example, sequential filtration through filters (e.g. 70- and 40-μm filters), essentially as described under “General Materials and Experimental Methods” of the Examples section which follows. Furthermore, mechanical dissociation of tissue into isolated cells can be performed using a device designed to break the tissue to a predetermined size. Such a device can be obtained from CellArtis Goteborg, Sweden. Additionally or alternatively, mechanical dissociation can be manually performed using a needle such as a 27 g needle (BD Microlance, Drogheda, Ireland) while viewing the tissue/cells under an inverted microscope. Following enzymatic or mechanical dissociation of the tissue, the dissociated cells are further broken to small clumps using 200 μl Gilson pipette tips (e.g., by pipetting up and down the cells).

Alternatively, such differentiated cells can be obtained by differentiation of stem cell. Hence, according to specific embodiments, the differentiated cells have been ex-vivo differentiated. Methods of inducing differentiation of stem cells are well known to the skilled in the art. Thus, for example approaches to differentiate pluripotent cells into cardiomyocytes are disclosed in e.g. Burridge et al. (2012) Cell Stem Cell 10:16-28; Mummery et al. Circ Res. 2012 Jul. 20; 111(3):344-58; WO2014200339, WO2011056416, WO2015004539, WO201202649 land WO2014078414 and U.S. Pat. No. 7,534,607, the contents of which are fully incorporated herein by reference.

It will be appreciated that commercially available differentiated cells can also be used according to some embodiments of the invention.

According to specific embodiments, the differentiated cells comprise cardiomyocytes.

According to specific embodiments, the differentiated cells comprise lymphocytes.

According to specific embodiments, the cells comprise bone marrow cells.

Methods of obtaining bone marrow cells are well known in the art. Thus, for example, bone marrow can be obtained from iliac crest, femora, tibiae, spine, rib or other medullar spaces. Following, the desired cellular fraction can be purified from the bone marrow aspirates.

According to specific embodiments, the cells comprise mononuclear bone marrow cells (mnBMCs).

According to specific embodiments, the cells are comprised in mononuclear bone marrow cells (mnBMCs).

According to specific embodiment, the cells comprise at least 50% at least 60%, at least 70%, at least 80%, at least 90% or at least 95% mnBMCs.

Thus, for example, the mononuclear fraction can be purified from the bone marrow aspirates. There are several methods and reagents known to those skilled in the art for purifying mnBMCs from bone marrow such as, density gradient centrifugation (e.g. ficoll), sedimentation (e.g. Hespan), centrifugal elutriation, fractionation, automated processes (e.g. Sepax® from Biosafe S A, Eysins, Switzerland and the AutoXpress Platform® (AXP) from Thermogenesis, Rancho Cordova, CA chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the bone marrow cells are obtained by density gradient centrifugation (e.g. ficoll) of bone marrow cells.

According to specific embodiments, the bone marrow cells comprise stem cells (e.g. hematopoietic stem cells, mesenchymal stem cells). Methods of obtaining bone marrow stem cells are well known in the art and are further described hereinbelow.

According to specific embodiments, the bone marrow cells comprise mesenchymal stem cells (MSCs) and lymphocytes or progenitors thereof.

According to specific embodiments, the bone marrow cells comprise lymphocytes or progenitors thereof.

As used herein the term “lymphocytes progenitors” refers to hematopoietic progenitor cells that can differentiate to lymphocytes.

According to a specific embodiment the lymphocytes progenitor cells are mobilized to the peripheral blood by agents such as G-CSF with or without chemotherapy.

According to specific embodiments, the cells comprise stem cells.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, such as embryonic cells within the first couple of cell divisions after fertilization are the only cells that can differentiate into embryonic and extra-embryonic cells and are able to develop into a viable human being. Preferably, the phrase “pluripotent stem cells” refers to cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPS). The multipotent stem cells include e.g. adult stem cells, hematopoietic stem cells and mesenchymal stem cells.

It will be appreciated that undifferentiated stem cells are of a distinct morphology, which is clearly distinguishable from differentiated cells of embryo or adult origin by the skilled in the art. Typically, undifferentiated stem cells have high nuclear/cytoplasmic ratios, prominent nucleoli and compact colony formation with poorly discernable cell junctions. Additional features of undifferentiated stem cells are further described hereinunder.

According to specific embodiments, the stem cells are selected from the group consisting of embryonic stem cells (ESCs), induced pluripotent stem cells (iPS), hematopoietic stem cells (HSCs) and mesenchymal stem cells (MSCs).

According to specific embodiments, the cells are embryonic stem cells (ESCs).

The phrase “embryonic stem cells (ESCs)” refers to ESCs which are capable of differentiating into cells of all three embryonic germ layers (i.e., endoderm, ectoderm and mesoderm), or remaining in an undifferentiated state. The phrase “embryonic stem cells (ESCs)” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

The ESCs of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ESCs the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92:7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ESCs can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

In addition, ESCs can be obtained from other species as well, including mouse (Mills and Bradley, 2001), golden hamster [Doetschman et al., 1988, Dev Biol. 127: 224-7], rat [Iannaccone et al., 1994, Dev Biol. 163:288-92] rabbit [Giles et al. 1993, Mol Reprod Dev. 36:130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ESCs is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

ESCs (e.g., human ESCs) originating from an unfertilized ova stimulated by parthenogenesis (parthenotes) are known in the art (e.g., Zhenyu Lu et al., 2010. J. Assist Reprod. Genet. 27:285-291; “Derivation and long-term culture of human parthenogenetic embryonic stem cells using human foreskin feeders”, which is fully incorporated herein by reference). Parthenogenesis refers to the initiation of cell division by activation of ova in the absence of sperm cells, for example using electrical or chemical stimulation. The activated ovum (parthenote) is capable of developing into a primitive embryonic structure (called a blastocyst) but cannot develop to term as the cells are pluripotent, meaning that they cannot develop the necessary extra-embryonic tissues (such as amniotic fluid) needed for a viable human fetus.

According to specific embodiments, the cells are embryonic stem cells Induced pluripotent stem cells (iPS).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell (2007) 131:861-872]. Other embryonic-like stem cells can be generated by nuclear transfer to oocytes, fusion with embryonic stem cells or nuclear transfer into zygotes if the recipient cells are arrested in mitosis.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Adult tissue stem cells can be isolated using various methods known in the art such as those disclosed by Alison, M. R. [J Pathol. (2003) 200(5): 547-50], Cai, J. et al., [Blood Cells Mol Dis. 2003 31(1): 18-27], Collins, A. T. et al., [J Cell Sci. 2001; 114(Pt 21): 3865-72], Potten, C. S. and Morris, R. J. [Epithelial stem cells in vivo. 1988. J. Cell Sci. Suppl. 10, 45-62], Dominici, M et al., [J. Biol. Regul. Homeost. Agents. 2001, 15: 28-37], Caplan and Haynesworth [U.S. Pat. No. 5,486,359] Jones E. A. et al., [Arthritis Rheum. 2002, 46(12): 3349-60]. Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62].

Hematopoietic stem cells (HSCs), which may also referred to as adult tissue stem cells, include stem cells obtained from blood or bone marrow tissue of an individual at any age or from cord blood of a newborn individual. According to specific embodiments, the cells comprise HSCs.

According to a specific embodiment, hematopoietic stem cell is a CD34+ cell.

As used herein the term “CD34+ cell” refers to a hematopoietic stem cell positive for the CD34 marker that can differentiate to each of the cell types in the blood, i.e.; the myeloid (monocyte, macrophage, neutrophil, basophil, eosinophil, erythrocyte, megakaryocyte, dendritic cell) or lymphoid (T cell, B cell, NK cell) lineages.

The HSCs may be a specific cell line, alternatively may be generated from iPS or embryonic stem cells [see for example Pick M et al. (2007) Stem Cells, 25(9): 2206-14; and Pick M et al. (2013) PLoS One, 8(2): e55530] or alternatively may be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton. Thus, for example, HSCs can be isolated from cord blood, peripheral blood or BM samples by means of density gradient centrifugation using for example Ficoll-Paque (can be obtained from GE Healthcare Bio-Science AB) followed by immunomagnetic or immunofluorescent methods (such as Diamond or Microbeads CD34+ isolation kit obtained from Miltenyi Biotech). Purity of the purified fraction can be assessed by flow cytometry for the specified markers (for example CD34).

According to a specific embodiment the HSCs are mobilized to the peripheral blood by agents such as G-CSF with or without chemotherapy.

Placental and cord blood stem cells may also be referred to as “young stem cells”.

Fetal stem cells can be isolated using various methods known in the art such as those disclosed by Eventov-Friedman S, et al., PLoS Med. 2006, 3: e215; Eventov-Friedman S, et al., Proc Natl Acad Sci USA. 2005, 102: 2928-33; Dekel B, et al., 2003, Nat Med. 9: 53-60; and Dekel B, et al., 2002, J. Am. Soc. Nephrol. 13: 977-90.

According to specific embodiments, the cells comprise mesenchymal stem cells (MSCs).

Mesenchymal stem cells give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the bone marrow far exceeds their abundance in other tissues and as such isolation from bone marrow is presently preferred.

Methods of isolating, purifying and expanding MSCs are known in the art and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

According to specific embodiments, MSC cultures are generated by diluting bone marrow aspirates (usually 20 ml) with equal volumes of Hank's balanced salt solution (HBSS; GIBCO Laboratories, Grand Island, NY, USA) and layering the diluted cells over about 10 ml of a Ficoll column (Ficoll-Paque; Pharmacia, Piscataway, NJ, USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, α medium without deoxyribonucleotides or ribonucleotides; GIBCO); 2-20% fetal calf serum (FCS) derived from a lot selected for rapid growth of MSCs (Atlanta Biologicals, Norcross, GA); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the MSC fraction, cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention include three groups of cells which are defined by their morphological features: small and agranular cells (referred to as RS-1, hereinbelow), small and granular cells (referred to as RS-2, hereinbelow) and large and moderately granular cells (referred to as mature MSCs, hereinbelow). The presence and concentration of such cells in culture can be assayed by identifying a presence or absence of various cell surface markers, by using, for example, immunofluorescence, in situ hybridization, and activity assays.

When MSCs are cultured under the culturing conditions of some embodiments of the invention they exhibit negative staining for the HSC markers CD34, CD11B, CD43 and CD45. A small fraction of cells (less than 10%) are dimly positive for CD31 and/or CD38 markers. In addition, mature MSCs are dimly positive for the HSC marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Stro1 markers and are dimly positive for the CD90 marker, and the RS-2 cells are negative for all of these markers.

The cells of some embodiments of the present invention can be a primary cell (non-cultured and alternatively or additionally non-immortalized cell) or a cell-line.

According to specific embodiments, the cells used and/or obtained according to the present invention can be freshly isolated, stored e.g., cryopreserved (i.e. frozen) at e.g. liquid nitrogen temperature at any stage (e.g. following their retrieval, following contacting with the agent, following treatment with TNFα) for long periods of time (e.g., months, years) for future use; and cell lines.

Thus, according to specific embodiments, the cells are cryopreserved cells.

According to specific embodiments, the cells are cryopreserved prior to contacting with the agent which downregulates expression and/or activity of TNFR1.

According to specific embodiments, the cells are cryopreserved following contacting with the agent which downregulates expression and/or activity of TNFR1.

According to specific embodiments, the cells are cryopreserved prior to treatment with TNFα.

According to specific embodiments, the cells are cryopreserved following treatment with TNFα.

According to specific embodiments, the cells are cryopreserved prior to administering to the subject.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the present invention can be stored in a cell bank or a depository or storage facility.

According to specific embodiments, the cells are freshly isolated (i.e., not subjected to preservation processes).

The cells used according to specific embodiments of the present invention may be autologous or non-autologous; they can be syngeneic or non-synegeneic: allogeneic or xenogeneic to the subject; each possibility represents a separate embodiment of the present invention.

According to specific embodiments, the cells are autologous to said subject.

As used herein, the term “autologous” means that the donor subject is the recipient subject. Thus, in autologous transplantation the cells have been removed and re-introduced e.g., re-infused to the subject.

According to specific embodiments, the cells are non-autologous to said subject.

As used herein, the term “non-autologous” means that the donor subject is not the recipient subject.

As used herein, the term “syngeneic” means that the donor subject is essentially genetically identical with the recipient subject. Examples of syngeneic transplantation include transplantation of cells derived from the subject (also referred to in the art as “autologous”), a clone of the subject, or a homozygotic twin of the subject

As used herein, the term “allogeneic” means that the donor is of the same species as the recipient, but which is substantially non-clonal with the recipient. Typically, outbred, non-zygotic twin mammals of the same species are allogeneic with each other. It will be appreciated that an allogeneic donor may be HLA identical or HLA non-identical with respect to the subject

As used herein, the term “xenogeneic” means that the donor subject is from a different species relative to the recipient subject.

According to specific embodiments, the cells are mammalian cells.

According to specific embodiments, the cells are primate cells.

According to specific embodiments, the cells are human cells.

According to other specific embodiments, the cells are rodent cells (e.g. mouse, rat).

The cells of some embodiments of the present invention are characterized by reduced level of expression and/or activity of TNFR1.

As used herein, the term “TNFR1 (Tumor necrosis factor receptor 1)”, also known as tumor necrosis factor receptor superfamily member 1A (TNFRSF1A), CD120a and p55 refers to the TNFR1 gene and to its polynucleotide or polypeptide expression product. According to specific embodiments, the TNFR1 refers to the human TNFR1, such as provided in Gene ID: 7132 (SEQ ID NO: 2) and the following Accession Numbers: NM_001065 (SEQ ID NO: 3), NM_001346091 (SEQ ID NO: 4), NM_001346092 (SEQ ID NO: 5), NP_001056 (SEQ ID NO: 6), NP_001333020 (SEQ ID NO: 7) and NP_001333021 (SEQ ID NO: 8). According to specific embodiments, the TNFR1 refers to the mouse TNFR1, such as provided in Gene ID: 21937 (SEQ ID NO: 9) and in the following Accession Numbers: NM_011609 (SEQ ID NO: 10) and NP_035739 (SEQ ID NO: 11).

According to specific embodiments, TNFR1 activity is at least one of (or two of or all of): binding TNFα, forming a trimer, activating the transcription factor NFκB and/or mediating apoptosis.

As used herein, “reduced level of expression and/or activity” refers to a decrease of at least 10% in TNFR1 expression or activity in comparison to a control cell of the same origin which was not contacted with an agent which downregulates expression and/or activity of TNFR1, as may be determined by e.g. PCR, ELISA, Western blot analysis, immunoprecipitation, flow cytometry, immuno-staining, TNFα signaling assays. According to a specific embodiment, the decrease is in at least 20%, 30%, 40% or even higher say, 50%, 60%, 70%, 80%, 90% or even 100%.

According to specific embodiments, the cell does not express TNFR1.

According to specific embodiments the cells with reduced expression and/or activity of TNFR1 are genetically modified cells. Methods and agents for genetically modifying cells are well known in the art and are further described in details hereinbelow.

According to specific embodiments, the cells are genetically modified with a CRISPR/Cas system, a Zinc finger nuclease (ZFN), transcription-activator like effector nuclease (TALEN) or meganuclease for downregulating expression of said TNFR1.

According to specific embodiments, the cells are genetically modified with a CRISPR/Cas system for downregulating expression of said TNFR1.

According to specific embodiments, the cells with reduced level of expression and/or activity of TNFR1 express similar levels and/or activity of TNFR2 in comparison to a control cell of the same origin which was not contacted with an agent which downregulates expression and/or activity of TNFR1, as may be determined by e.g. PCR, ELISA, Western blot analysis, immunoprecipitation, flow cytometry, immuno-staining, TNFα signaling assays.

As used herein, the term “TNFR2 (Tumor necrosis factor receptor 2)”, also known as tumor necrosis factor receptor superfamily member 1B (TNFRSF1B) and CD120b and p75 refers to the TNFR2 gene and to its polynucleotide or polypeptide expression product. According to specific embodiments, the TNFR2 refers to the human TNFR2, such as provided in Gene ID: 7133 (SEQ ID NO: 12) and the following Accession Numbers: NM_001066 (SEQ ID NO: 13) and NP_001057 (SEQ ID NO: 14). According to specific embodiments, the TNFR1 refers to the mouse TNFR2, such as provided in Gene ID: 21938 (SEQ ID NO: 15) and in the following Accession Numbers: NM_011610 (SEQ ID NO: 16) and NP_035740 (SEQ ID NO: 17).

According to specific embodiments, TNFR2 activity is at least one of (or two of or all of): binding TNFα and/or mediating the recruitment of two anti-apoptotic proteins, c-IAP1 and c-IAP2.

As the cells of some embodiments of the present invention are characterized by reduced level of expression and/or activity of TNFR1, the present invention also contemplates methods of obtaining such cells.

Thus, according to an aspect of the present invention there is provided a method of downregulating expression and/or activity of TNFR1 in differentiated cells or bone marrow stem cells, the method comprising:
(i) contacting ex-vivo or in-vitro differentiated cells or bone marrow stem cells with an agent which downregulates expression and/or activity of TNFR1; and
(ii) contacting ex-vivo or in-vitro said cells with TNFα.

According to another aspect of the present invention there is provided a method of downregulating expression and/or activity of TNFR1 in differentiated cells, the method comprising:
(i) inducing ex-vivo or in-vivo differentiation of stem cells;
(ii) contacting ex-vivo or in-vitro said cells with an agent which downregulates expression and/or activity of TNFR1; and
(iii) contacting ex-vivo or in-vitro said cells with TNFα.

According to specific embodiments, inducing differentiation of the stem cells is effected prior to contacting with the agent. Methods of inducing differentiation of stem cells are known in the art and are further described hereinabove.

According to another aspect of the present invention there is provided a method of downregulating expression and/or activity of TNFR1 in differentiated cells or bone marrow stem cells, the method comprising genetically modifying ex-vivo or in-vitro differentiated cells or bone marrow stem cells with an agent which downregulates expression and/or activity of TNFR1.

According to specific embodiments, the method further comprising contacting the cells ex-vivo or in-vitro with TNFα.

According to specific embodiments, contacting the cells with the agent is effected prior to contacting the cells with the TNFα.

According to specific embodiments, contacting the cells with the agent is effected following contacting the cells with the TNFα.

Specific embodiments of the present invention contemplates a method of treating an ischemic disease in a subject in need thereof, the method comprising:
(i) obtaining cells according to the methods of downregulating expression and/or activity of TNFR1 described herein; and
(ii) administering to the subject a therapeutically effective amount of said cells, thereby treating the ischemic disease in the subject.

As used herein, “downregulating expression and/or activity” and “downregulates expression and/or activity” refer to a decrease of at least 5% in expression and/or biological function in the presence of the agent in comparison to same in the absence of the agent, as determined by e.g. PCR, ELISA, Western blot analysis, immunoprecipitation, flow cytometry, immuno-staining, TNFα signaling assays. According to a specific embodiment, the decrease is in at least 10%, 30%, 40% or even higher say, 50%, 60%, 70%, 80%, 90% or even 100%. According to specific embodiments, the decrease is at least 1.5 fold, at least 2 fold, at least 3 fold, at least 5 fold, at least 10 fold, or at least 20 fold as compared to same in the absence of the agent.

According to specific embodiments, downregulating expression refers to the absence of mRNA and/or protein, as detected by RT-PCR or Western blot, respectively.

According to specific embodiments, “downregulating expression and/or activity of TNFR1” and “downregulates expression and/or activity of TNFR1” refer to the ability to specifically downregulate the expression and/or activity of TNFR1 and not to downregulate the expression and/or activity of TNFR2, as determined by e.g. PCR, ELISA, Western blot analysis, immunoprecipitation, flow cytometry, immuno-staining, TNFα signaling assays. This selective inhibition can be manifested as higher affinity (e.g., Ke) of the agent to TNFR1 than to TNFR2. Increased affinity can be of at least 5, 10, 100, 1000 or 10000 fold.

Hence, according to specific embodiments, the agent does not downregulate expression and/or activity of TNFR2.

Downregulating expression and/or activity can be effected at the genomic (e.g. homologous recombination and site specific endonucleases) and/or the transcript level using a variety of molecules which interfere with transcription and/or translation (e.g., RNA silencing agents) of a target expression product described herein, but may also be effected at the protein level (e.g., antibodies, small molecules, inhibitory peptides, enzymes that cleave the polypeptide, aptamers and the like).

According to specific embodiments, the agent directly binds TNFR1.

According to other specific embodiments, the agent indirectly binds TNFR1 by acting through an intermediary molecule, for example the agent binds to or modulates a molecule that in turn binds to or modulates the target.

According to specific embodiments, the agent does not bind TNFR2.

Non-limiting examples of down-regulating agents at the nucleic acid level are described in details hereinbelow.

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Downregulation can be achieved by inactivating the gene (i.e. the TNFR1 gene) via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the TNFR1 may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of gRNA sequences that can be used with some embodiments of the present invention are described in Tables 1A and 1B hereinbelow.

According to specific embodiments, the gRNA sequence does not have a significant off target effect. Methods of determining off target effect are well known in the art, such as BGI Human Whole Genome Sequencing (described in Nature; 491:65-56.2012), next generation sequencing (NGS) using e.g. commercially available kits such as Alt-R-Genom Editing (IDT detection kit) or Sure select target enrich<1% variant allele frequency (Agilent).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Alternatively, the target cell can be transfected with both gRNA and Cas9 without plasmid using e.g. a transfection reagent such as CRISPRMAX [see e.g. Yu et al. (2016) JD1Biotechnol Lett. 38(6):919-29]. In some cells electroporation can improve the transfection of the gRNA and the Cas9 [see e.g. Liang et al. (2015) Journal of Biotechnology 208, 2015, Pages 44-53; and Liang et al. (2017) Journal of Biotechnology, Volume 241, 2017, pp. 136-146].

TABLE 1A
TNFR1 guide RNA sequences designed according to
the mouse TNFR1

| | | | | GCD |
|---|---|---|---|---|
| | Sequence | | | Band |
| | of gRNA | Forward | Reverse | sizes |
| CRISPR1 | CGGAC | GAACA | TTCGA | 604 |
| | AGTCA | ATTCC | AATCC | 378 bp |
| | CTCAC | ATCTG | TGACC | 226 bp |
| | CAAGT | CTGCA | TCCTG | |
| | SEQ ID | CC | G | |
| | NO: 18 | SEQ ID | SEQ ID | |
| | | NO: 19 | NO: 20 | |
| CRISPR2 | GACAC | TGAGA | CATGG | 600 |
| | TGCCT | CAGGG | CATTC | 357 bp |
| | GAGGT | TTTCT | GTCTT | 243 bp |
| | AATTC | CTATA | TGT | |
| | SEQ ID | CC | SEQ ID | |
| | NO: 21 | SEQ ID | NO: 23 | |
| | | NO: 22 | | |
| CRISPR3 | CGGCT | AGTGA | GAGCT | 608 |
| | TCCCA | GAGCA | TGCAT | 417 bp |
| | GAATT | GAGAA | GTGCA | 191 bp |
| | ACCTC | TTGTC | CGCAT | |
| | SEQ ID | SEQ ID | SEQ ID | |
| | NO: 24 | NO: 25 | NO: 26 | |

*Mouse tumor necrosis factor receptor superfamily, member 1a mRNA: NM_011609 (SEQ ID NO: 10) Total No. of Exons: 10, Targeted Exons: 2 (2, 3)

TABLE 1B
TNFR1 guide RNA sequences designed according to
the human TNFR1

| | Sequence | | | GCD |
|---|---|---|---|---|
| | of | | | Band |
| | gRNA | Forward | Reverse | sizes |
| CRISPR1 | ATATA | TCTCA | CAGAT | 625 |
| | CCCCT | CACTC | TGTAT | 415 bp |
| | CAGGG | CCTGC | GGCCC | 210 bp |
| | GTTAT | AGTCC | CAACT | |
| | SEQ ID | GT | GT | |
| | NO: 27 | SEQ ID | SEQ ID | |
| | | NO: 28 | NO: 29 | |
| CRISPR2 | ATTGG | TTCTG | CAGAT | 648 |
| | ACTGG | AAGCG | TGTAT | 420 bp |
| | TCCCT | GTGAA | GGCCC | 228 bp |
| | CACCT | GGAGC | CAACT | |
| | SEQ ID | C | GT | |
| | NO: 30 | SEQ ID | SEQ ID | |
| | | NO: 31 | NO: 32 | |
| CRISPR3 | TACTT | TGCCG | GGGTG | 484 |
| | GTACA | GTACT | CTGCT | 360 bp |
| | ATGAC | GGTTC | TCTTT | 124 bp |
| | TGTCC | TTCCT | CTCTG | |
| | SEQ ID | G | CT | |
| | NO: 33 | SEQ ID | SEQ ID | |
| | | NO: 34 | NO: 35 | |
| CRISPR4 | TAATG | ACCAC | TCACC | |
| | TATCG | AGTGC | TCCCT | |
| | CTACC | TGTTG | CCACA | |
| | AACGG | CC | CAT | |
| | SEQ ID | SEQ ID | SEQ ID | |
| | NO: 36 | NO: 37 | NO: 38 | |
| CRISPR5 | CACTC | ATCTC | ACGGT | |
| | CAATA | TTCTT | GTTCT | |
| | ATGCC | GCACA | GTTTC | |
| | GGTAC | GTGGA | TCCTG | |
| | SEQ ID | C | SEQ ID | |
| | NO: 39 | SEQ ID | NO: 41 | |
| | | NO: 40 | | |
| CRISPR6 | AGAGG | GAAGA | GTTGT | |
| | TGCAC | ACCAG | CAGAC | |
| | GGTCC | TACCG | CCACA | |
| | CATTG | GCATT | GAATA | |
| | SEQ ID | A | C | |
| | NO: 42 | SEQ ID | SEQ ID | |
| | | NO: 43 | NO: 44 | |
| CRISPR7 | TTGGA | TCTCT | ACTGG | |
| | CTGGT | TGATG | AAGAA | |
| | CCCTC | GTGTC | GCAGA | |
| | ACCTA | TCCTC | GAAAG | |
| | SEQ ID | TA | AA | |
| | NO: 45 | SEQ ID | SEQ ID | |
| | | NO: 46 | NO: 47 | |

*Human tumor necrosis factor receptor superfamily, member 1a mRNA: NM_001065 (SEQ ID NO: 3)

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. Dec 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of the target may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Downregulation can also be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (i.e. TNFR1) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433; and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation can be effected using an antisense polynucleotide capable of specifically hybridizing with an mRNA transcript encoding the target (i.e. TNFR1).

Design of antisense molecules which can be used to efficiently downregulate a target must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jäaskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

According to specific embodiments, the agent is selected from the group consisting of CRISPR/Cas system, a Zinc finger nuclease (ZFN), transcription-activator like effector nuclease (TALEN), meganuclease, antisense and siRNA.

According to specific embodiments, down-regulating expression and/or activity is effected at the genomic level.

According to specific embodiments, the agent comprises a CRISPR/Cas system.

Embodiments of the invention further contemplate the use of the CRISPR/Cas system per se (as the therapeutic agent and not as a cell therapy) for downregulating expression of TNFR1 for treating an ischemic disease.

Thus, according to an aspect of the present invention, there is provided a method of treating an ischemic disease in a subject in need thereof, the method comprising administering to the subject a therapeutically effective amount of a CRISPR/Cas system for downregulating expression of TNFR1, thereby treating the ischemic disease in the subject.

Such CRISPR/Cas systems are described in details hereinabove.

According to specific embodiments, the CRISPR/Cas system does not downregulate expression of TNFR2.

The cells or the agents (e.g. a CRISPR/Cas system for downregulating expression of TNFR1) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cell or the agent (e.g. a CRISPR/Cas system for downregulating expression of TNFR1) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to specific embodiments, the cells or the agent (e.g. a CRISPR/Cas system for downregulating expression of TNFR1) or the pharmaceutical composition comprising same is administered intravenously.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the cells or the agent (e.g. a CRISPR/Cas system for downregulating expression of TNFR1) or the pharmaceutical composition comprising same in a local rather than systemic manner, for example, via injection of the pharmaceutical composition directly into a tissue region of a patient.

According to specific embodiments, the cells or the agent (e.g. a CRISPR/Cas system for downregulating expression of TNFR1) or the pharmaceutical composition comprising same is administered into the ischemic tissue.

According to specific embodiments, the cells or the agent (e.g. a CRISPR/Cas system for downregulating expression of TNFR1) or the pharmaceutical composition comprising same is administered by transendocardial injection.

According to specific embodiments, the cells or the agent (e.g. a CRISPR/Cas system for downregulating expression of TNFR1) or the pharmaceutical composition comprising same is administered intracoronary or intramyocardialy.

According to specific embodiments, the cells or the agent (e.g. a CRISPR/Cas system for downregulating expression of TNFR1) or the pharmaceutical composition comprising same is attached to an implantable device such as a stent, a valve, an intravascular device and a pacemaker.

According to some embodiments, the cells of the present invention are administered in a hydrated gel (hydrogel) such as a hyaluronic acid-based hydrogels (see the Examples section which follows). Such hydrogels are known in the art and disclosed e.g. in Xu et al. (2012) Soft Matter. 8(12):3280-3294; Kim et al. (2012) Knee Surg Relat Res. September; 24(3): 164-172, the contents of which are fully incorporated herein by reference.

According to specific embodiments, the hyaluronic acid is provided at a concentration range of about 0.1-10%, e.g., about 0.5-10%, e.g., about 0.5-5, e.g., about 1-5%, e.g., about 2-5%, e.g., about 3-5%, e.g., about 3-4% in the composition e.g. hydrogel.

According to a specific embodiment, the hyaluronic acid is provided at a concentration range of about 3-4% in the composition e.g. hydrogel.

According to some embodiments, the cells of the present invention are administered in a biodegradable co-polymer or scaffold. Such scaffolds are known in the art and disclosed e.g. in Florian Weinbergeret al. (2017) Circulation Research. 120:1487-1500; Rochkind S et al (2004) Neurol Res. 26(2):161-6; Rochkind S. et al. (2006) Eur Spine J. 15(2):234-45; and F S Y Wong, A C Y Lo (2015) J Stem Cell Res Ther 5:267, the contents of which are fully incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the cells, the agent (e.g. a CRISPR/Cas system for downregulating expression of TNFR1) effective to prevent, alleviate or ameliorate symptoms of a disorder (e.g., ischemic disease, e.g., ischemic heart disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient in levels sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

According to specific embodiments, the cells, the agent (e.g. a CRISPR/Cas system for downregulating expression of TNFR1) or the pharmaceutical composition comprising same are administered within 1.5-24 hours following diagnosis of said ischemic disease.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of the present invention, there is provided a pharmaceutical composition comprising as an active ingredient differentiated cells or bone marrow stem cells genetically modified with a CRISPR/Cas system for downregulating expression of TNFR1.

According to another aspect of the present invention, there is provided a pharmaceutical composition comprising as active ingredients cells genetically modified with a CRISPR/Cas system for downregulating expression of TNFR1 and at least 2 ng/ml TNFα.

According to another aspect of the present invention there is provided an article of manufacture comprising TNFα and the cells disclosed herein with reduced expression and/or activity of TNFR1 as compared to control cells of the same origin not contacted with an agent which downregulates expression and/or activity of said TNFR1.

According to specific embodiments, the TNFα and the cells are in a co-formulation.

According to specific embodiments, the TNFα and the cells are in separate containers.

According to another aspect of the present invention there is provided an article of manufacture comprising TNFα and a CRISPR/Cas system for downregulating expression of TNFR1.

According to specific embodiments, the TNFα and a CRISPR/Cas system are in a co-formulation.

According to specific embodiments, the TNFα and a CRISPR/Cas system are in separate containers.

According to specific embodiments, TNFα is provided at a concentration above its physiological concentration in the cells.

According to specific embodiments, TNFα is provided at a concentration of at least 2 ng/ml, at least 5 ng/ml, at least 10 ng/ml, at least 20 ng/ml, at least 30 ng/ml, at least 40 ng/ml or at least 50 ng/ml, each possibility represents a separate embodiment of the present invention.

According to specific embodiments, TNFα is provided at a concentration of 2-200 ng/ml or 20-200 ng/ml.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Mouse mnBMCs Transfected with Mouse TNFR1 Crispr Reduce Infarct Size in a Myocaridal Nfarction Mouse Model

### Materials and Methods

Isolation of mononuclear bone marrow cells—Seven 8 weeks old C57Bl/6 (OldHsd) male mice weighing 20-23 gram were purchased from Envigo RMS (Israel) LTD. Bone marrow (BM) was flushed from tibiae and femurs of each mouse using 2 mL PBS supplemented with 2% fetal bovine serum (FBS). The suspension was resuspended and mashed through a 40-100 μM cell strainer. The sample was centrifuged at 300 g for 5 minutes and resuspended in 100 μL PBS. Following, 1 mL of red blood cell (RBC) lysis buffer (Roche) was added and the sample was incubated for 3 minutes at room temperature. 9 mL PBS (3 g/L glucose) supplemented with 2% FBS (3 g/L glucose) was added to stop the reaction and the sample was centrifuged at 300 g for 5 minutes and immediately resuspended in 1 mL of ice cold PBS supplemented with 2% fetal bovine serum (FBS). Mononuclear bone marrow cells (mnBMCs) were separated from the BM sample by Ficoll-Paque (Histopaque1077, Sigma), with 3 technical replicates. BM cells were resuspended in 2 mL PBS supplemented with 2% murine serum were layered on 1.5 ml Ficoll-Paque. The Ficoll-Paque gradients were centrifuged for 40 minutes at 400 g without brake. The mnBM layer was then collected, washed in PBS supplemented with 2% FBS. Viability and cell count was determined by trypan blue exclusion method using hemocytometer. A total of 100×10⁶ mnBMCs was counted. 60×10⁶mnBMCs were frozen at −80° C. 40×10⁶ cells were seeded in 24 wells plates (120,000 mnBMCs per well) in 1 mL/well DMEM medium containing—glutamine, sodium pyruvate, pen/strep and 3% mouse serum and cultured under standard culture conditions. Following 24 hours of incubation, the medium was replaced with optimem medium (Invitrogen) and cells were transfected with TNFR1 CRISPR for 2 days. Control cells, which were not transfected with TNFR1 CRISPR were cultured under the same culturing conditions: 120,000 mnBMCs per well in 24 wells plates in DMEM medium containing 2% FBS.

TNFR1 CRISPR transfection—For CRISPR transfection three different mouse TNFR1 guide RNA were designed and synthesized (Table 1A hereinabove) and mnBMCs were transfected with case 9 protein with the lipophilic transfection reagent CRISPRMAX as described before (Yu et al 2016).

mnBM Transplantation—750,000 cultured mnBMCs were treated with 30 ng/ml mouse TNFα (PeproTech) for 20 minutes and added to 50 μL gel containing 3.75% hyaluronic acid (HA), 20% non-activated murine serum (serum obtained from C57BL/6 mice and inactivated in 56° C.) in DMEM. Following ligation of the left anterior descending artery (LAD), 50 μL gel containing mnBMCs were injected to the heart as described in Toma et al 2002 and Kawada et al 2016.

Animal model—Animal experiments were conducted according to the guidelines of the Animal Care and Use Committee of Israel, and the Guide for the Care and Use of Laboratory Animals published by the US National Institute of Health. 18 C57BL/6 mice were purchased at 8 weeks (20-23 gr) from Ex-Vivo Israel. Following 1 week of acclimation the mice were intubated and anesthetized with 0.5% isoflurane gas. Permanent ligation of the left anterior descending artery (LAD) was performed as described in Kolk et al. (2009) JoVE. 32 (ref 41). The mice were randomly assigned into three groups, see table 2 hereinbelow:
Group 1M—on day 1, 2 hours prior to LAD mice were injected IP with 7.5 μg TNFα (PeproTech) and following LAD mice were injected to the LAD area (estimated area of the scar formation) with 750,000 mnBMCs transfected with TNFR1 CRISPR, n=6;
Group 2M—on day 1, following LAD, mice were injected to the LAD area with 750,000 mnBMCs transfected with TNFR1 CRISPR, n=12;
Group 3M—on day 1, following LAD, mice were injected to the LAD area with 750,000 mnBMCs that were not transfected with TNFR1 CRISPR, n=6.

Injected mnBMCs in all groups (1M, 2M, 3M) were treated for 20 minutes prior to injection with 30 ng/mL mouse TNFα (PeproTech) containing 1 mg/ml murine serum.

Body weight, mortality and morbidity were evaluated once a day. Mice were sacrificed 24 hours or 4 days following LAD occlusion and ˜200 μl whole blood was collected from the retro-orbital sinus into yellow cap serum tubes. The collected blood was centrifuged (at 3000 rpm for 10 minutes) for serum preparation. Samples were frozen at (−60° C.)-(−90° C.) and stored in appropriately labeled test-tubes for further Creatin Kinase (CK) detection (Sigma). From each sacrificed mouse heart was dissected, weighed, fixated and sent for histological evaluation.

TABLE 2
Experimental design:

| Group | Mouse No. | Time | | Treatment |
|---|---|---|---|---|
| 1M | 3 | 24 | hours | TNFα IP + Injection of mnBMCs |
| | 5 | | | transfected with TNFR1 |
| | 6 | | | CRISPR and treated with TNFα |
| 2M | 7 | | | Injection of mnBMCs |
| | 9 | | | transfected with TNFR1 |
| | 10 | | | CRISPR and treated with TNFα |
| 3M | 15 | | | Control: Injection of non- |
| | 16 | | | transfected mnBMCs treated |
| | 18 | | | with TNFα |
| 1M | 1 | 4 | days | TNF IP + Injection of mnBMCs |

| | | |
|---|---|---|
| | 2 | transfected with TNFR1 |
| | | CRISPR and treated with TNFα |
| 2M | 11 | Injection of mnBMCs transfected |
| | 12 | with TNFR1 CRISPR and treated |
| | | with TNFα |
| 3M | 13 | Control: Injection of non-transfected |
| | 14 | mnBMCs treated with TNFα |

Histology—Hearts were harvested and fixed in 10% formaldehyde at Pharmaseed and transferred to Patho-Logica. All tissues were trimmed in the same manner into block cassettes. Transverse cross sections were performed in each heart producing four equal cross sections per organ. The tissues were embedded in paraffin, sectioned at no more than 5 micron thickness, and stained with Hematoxylin & Eosin (H&E) and Masson Trichrome (MT), to trace the injected cells, pathological changes and to evaluate the volume of the induced infarcts. Furthermore, to evaluated viability an apoptosis TUNEL kit stain was performed. The slides were photographed using a microscope (Olympus BX60), at magnifications of X10, X20 and X40, equipped with an Olympus DP-73 camera.

The slides were examined by a pathologist, according to the following parameters:

### H&E and MT Stains:

Infarct diameter (mm);
Presence of injected mnBMCs (−, +, ++, +++);
Additional pathological changes

### Scoring System for TUNEL Evaluation:

Grade: −, no signs of positive TUNEL expression in all tissues and cells.
Grade: +, Only few cells (<10) per cross section are positive.
Grade ++, More cells (>10, >50) per cross section are positive.
Grade +++, Many cells (>50) per cross section are positive.

### Results

The effect of transplantation of stem cells with reduced expression of TNFR1 on ischemic heart disease was assessed in a permanent ligation of the left anterior descending artery (LAD) mouse model, a known model of infarction and myocardial ischemia [Kolk et al. (2009) JoVE. 32]. To this end, mnBMCs transfected with TNFR1 CRISPR and treated with TNFα were injected to the LAD area and their effect on infract volume and cellular response was compared to the effect of injection of control mnBMCs (non-transfected and treated with TNFα).

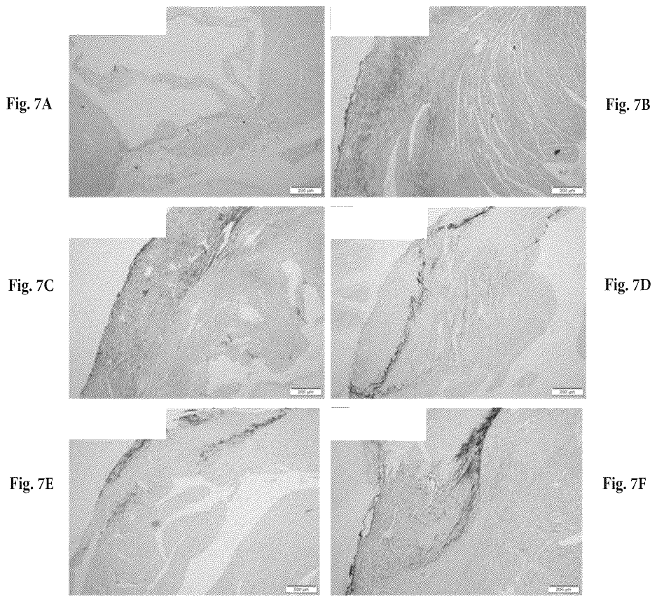

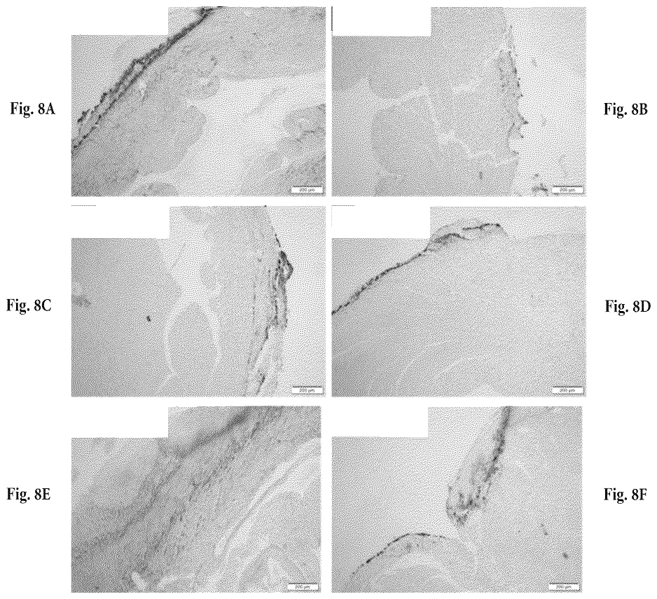

Histological evaluation of hearts extracted from LAD occluded mice injected with mnBMCs transfected with TNFR1 CRISPR demonstrated reduced infarct size (both width and depth), as determined 24 hours and 4 days following LAD occlusion (Group 2M) compared to control mice that were injected with non-transfected mnBMCs (Group 3M) (FIGS. 1A-2F, 4A-5D and 9A-9B and Table 3 hereinbelow). Interestingly, moderate to high TUNEL reaction in the infarct lesion was demonstrated in these mice (FIGS. 7A-8F), mainly localized in the inner part of the ventricle wall.

Ischemic preconditioning (IP) has been recognized as one of the most potent mechanisms to protect against myocardial ischemic injury. In experimental animals and humans, a brief period of ischemia has been shown to protect the heart from more prolonged episodes of ischemia, reducing infarct size, attenuating the incidence, and severity of reperfusion-induced arrhythmias, and preventing endothelial cell dysfunction (e.g. 6). It has been shown that classical preconditioning can be mimicked by administration of TNFα [9-13].

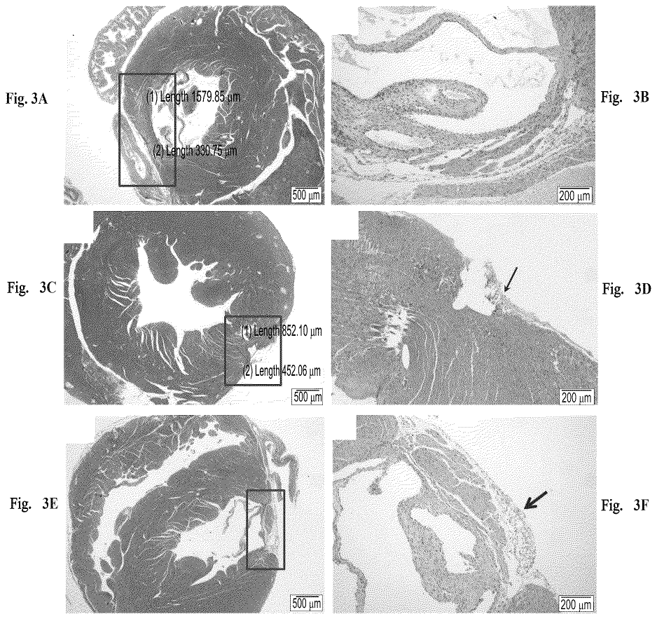

In the present model, treatment of the mice with TNFα 2 hours prior to LAD occlusion followed by injection of the TNFR1 CRISPR transfected mnBMCs (Group 1M) did not have a beneficial effect on infarct size. On the contrary, histological evaluation of hearts extracted from these mice revealed a large infarct volume with a relative low cellular reaction and mild to moderate TUNEL reaction in the infarct lesion, as detected 24 hours and 4 days following LAD (FIGS. 3A-3F, 6A-6D, 7A-8F and Table 3 hereinbelow).

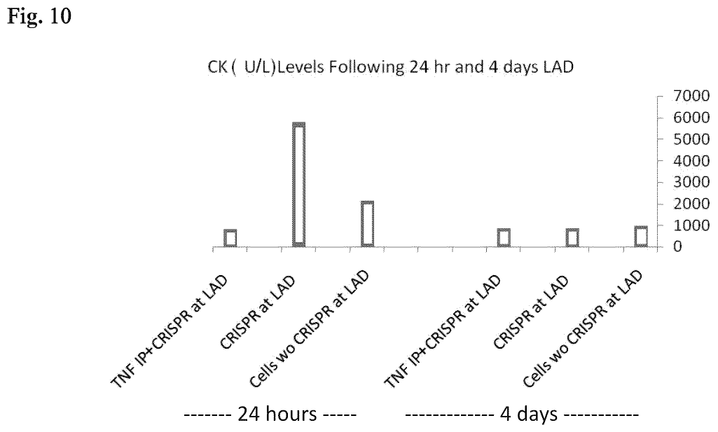

To evaluate toxicity, CK were determined in the serum of the transplanted mice. CK (made up of three enzyme forms, including CK-MB) levels can rise following heart attack, skeletal muscle injury and strenuous exercise. Importantly, detection of CK levels in the serum indicated that injection of the mnBMCs (either transfected or not transfected with TNFR1 CRISPR) did not have an effect of CK levels 4 days following LAD occlusion (FIG. 10).

Taken together, the results indicated a cardio-protective effect of transplantation of mnMB cells transfected with TNFR1 CRISPR against ischemic damage as demonstrated by reduced infarct depth and width in the mouse LAD model.

TABLE 3
Histological results of cardiac infarct parameters

| | | | Infarct volume | BM | | |
|---|---|---|---|---|---|---|
| | Animal | | mm (MT) | cells in | TUNNEL | Additional pathological |
| | # | Group | Depth/width | infarct | reaction | changes |
| 24 | 3 | 1M | Transmural/ | − | − | Few neutrophils and |
| hours | | | 1.57 | | | lymphocytes |
| post | 5 | 1M | 0.85/0.45 | − | − | Moderate infiltration of |
| LAD | | | | | | lymphocytes and few |
| | | | | | | neutrophils. Fibrin on top of |
| | | | | | | the infarct. |
| | 6 | 1M | Transmural/ | − | ++ | Almost no reaction |
| | | | 1.12 | | | |
| | 7 | 2M | 0.22/1.76 | − | +++ | Mild infiltration of mainly |
| | | | | | | lymphocytes and |
| | | | | | | macrophages. |
| | 9 | 2M | 0.59/1.37 | + | +++ | Macrophages and |
| | | | | | | neutrophils focally |
| | | | | | | infiltrated next to injected |
| | | | | | | cells debris |
| | 10 | 2M | Transmural/ | − | +++ | Cellular infiltration of |
| | | | 1.49 | | | neutrophils and lymphocytes |
| | | | | | | in the inner part of the |
| | | | | | | ventricle wall |
| | 15 | 3M | 0.37/0.71 | ++ | ++ | Macrophages and |
| | | | | | | neutrophils focally |
| | | | | | | infiltrated next to injected |
| | | | | | | cells debris |
| | 16 | 3M | 0.44/1.58 | ++ | +++ | Macrophages and |
| | | | | | | neutrophils focally |
| | | | | | | infiltrated next to injected |
| | | | | | | cells debris and some |
| | | | | | | adipocytes |
| | 18 | 3M | 0.32/0.88 | + | +++ | Macrophages and |
| | | | | | | neutrophils focally |
| | | | | | | infiltrated next to injected |
| | | | | | | cells debris |
| 4 | 1 | 1M | Transmural | − | +++ | A very large infarct with a |
| days | | | (2.02)/3.29 | | | central necrotic core, |
| post | | | | | | surrounded by many |
| LAD | | | | | | macrophages, lymphocytes |
| | | | | | | and some fibrocytes. |
| | 2 | 1M | Transmural | − | ++ | A very large infarct with a |
| | | | (1.06)/4.74 | | | central necrotic core, |
| | | | | | | surrounded by many |
| | | | | | | macrophages, lymphocytes |
| | | | | | | and some fibrocytes. |
| | 11 | 2M | Transmural | − | ++ | A very large infarct with a |
| | | | (1.14)/2.74 | | | central necrotic core, |
| | | | | | | surrounded by many |
| | | | | | | macrophages, lymphocytes |
| | | | | | | and some fibrocytes. |
| | 12 | 2M | Transmural | − | ++ | A very large infarct with a |
| | | | (1.01)/4.14 | | | central necrotic core, |
| | | | | | | surrounded by many |
| | | | | | | macrophages, lymphocytes |
| | | | | | | and some fibrocytes. |
| | 13 | 3M | 1.77/4.36 | − | +++ | A very large infarct with a |
| | | | | | | central necrotic core, |
| | | | | | | surrounded by many |
| | | | | | | macrophages, lymphocytes |
| | | | | | | and some fibrocytes. |
| | 14 | 3M | 2.02/3.99 | − | ++ | A very large infarct with a |
| | | | | | | central necrotic core, |
| | | | | | | surrounded by many |
| | | | | | | macrophages, lymphocytes |
| | | | | | | and some fibrocytes. |

### Example 2

### Human mnBMCs Transfected with Human TNFR1 CRISPR Reduce Infarct Size in a Myocaridal Nfarction Mouse Model

The effect of transplantation of human stem cells with reduced expression of TNFR1 on ischemic heart disease is assessed in a LAD occluded immune-deficient mouse model. To this end, human mnBMCs transfected with human TNFR1 CRISPR and treated with TNFα are injected to the LAD area and their effect on infract volume and cellular response is compared to the effect of injection of control human mnBMCs (non-transfected and treated with TNFα).

### Materials and Methods

Isolation of human mnBMCs—Bone marrow (BM) is flushed from tibiae, femurs or from peripheral blood or taken from human blood bank or human umbilical cord blood bank. Each sample is handled using 2 mL PBS supplemented with 2% human serum. The suspension is resuspended and mashed through a 40-100 μM cell strainer. The sample is centrifuged at 300 g for 5 minutes and re-suspended in 100 μL PBS. Following, 1 mL of red blood cell (RBC) lysis buffer (Roche) is added and the sample is incubated for 3 minutes at room temperature. 9 mL PBS (3 g/L glucose) supplemented with 2% human serum (3 g/L glucose) is added to stop the reaction and the sample is centrifuged at 300 g for 5 minutes and immediately re-suspended in 1 mL of ice cold PBS supplemented with 2% human serum. Mononuclear bone marrow cells (mnBMCs) are separated from the BM sample by Ficoll-Paque (Histopaque1077, Sigma), with 3 technical replicates, as follows: BM cells re-suspended in 2 mL PBS supplemented with 2% human serum are ayered on 1.5 ml Ficoll-Paque. The Ficoll-Paque gradients are centrifuged for 40 minutes at 400 g without brake. Following, the mnBM layer is collected, washed in PBS supplemented with 2% human serum. Viability and cell count is determined by trypan blue exclusion method using hemocytometer. A fraction of the mnBMCs are frozen at −80° C. Another fraction is seeded in 24 wells plates (120,000 mnBMCs per well) in 1 mL/well DMEM medium containing—glutamine, sodium pyruvate, pen/strep and 3% human serum and cultured under standard culture conditions. Following 1-24 hours of incubation, the medium is replaced with optimem medium (Invitrogen) and cells are transfected with TNFR1 CRISPR for 5 minutes up to 2 days or immediately electroporated. Control cells, which are not transfected with TNFR1 CRISPR are cultured under the same culturing conditions.

TNFR1 CRISPR transfection—For CRISPR transfection seven different human TNFR1 guide RNA were designed and synthesized (Table 1B hereinabove). mnBMCs are transfected with case 9 protein with the lipophilic transfection reagent CRISPRMAX as described before (Yu et al 2016) or transfected immediately by electroporation.

mnBM Transplantation—As described in Example 1 hereinabove.

Animal model—As described in Example 1 hereinabove with Nude and/or SCID mice instead of C57BL/6 mice.

Histology—As described in Example 1 hereinabove

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited Throughout the Application)

1 Beutler, B. and van Huffel, C. (1994) Unraveling function in the TNF ligand and receptor families. Science. 264, 667-668
2 Kolesnick, R. and Golde, D. W. (1994) The sphingomyelin pathway in tumor-necrosis-factor and interleukin-1 signaling. Cell. 77, 325-328
3 Beyaert, R. and Fiers, W. (1994) Molecular mechanisms of tumor necrosis factor-induced cytotoxicity—What we do understand and what we do not. FEBS Lett. 340, 9-16
4 Valgimigli, M., Ceconi, C., Malagutti, P., Merli, E., Soukhomovskaia, O., Francolini, G., Cicchitelli, G., Olivares, A., Parrinello, G., Percoco, G., Guardigli, G., Mele, D., Pirani, R. and Ferrari, R. (2005) Tumor necrosis factor-alpha receptor 1 is a major predictor of mortality and new-onset heart failure in patients with acute myocardial infarction—The cytokine-activation and long-term prognosis in myocardial infarction (C-ALPHA) study. Circulation. 111, 863-870
5 Kleinbongard, P., Schulz, R. and Heusch, G. (2010) TNFalpha in myocardial ischemia/reperfusion, remodeling and heart failure. Heart Fail Rev. 16, 49-69
6 Murry, C. E., Jennings, R. B. and Reimer, K. A. (1986) Preconditioning with ischemia: a delay of lethal cell injury in ischemic myocardium. Circulation. 74, 1124-1136
7 Marber, M. S., Latchman, D. S., Walker, J. M. and Yellon, D. M. (1993) Cardiac stress protein elevation 24 hours after brief ischemia or heat stress is associated with resistance to myocardial infarction. Circulation. 88, 1264-1272
8 Schulz, R., Gres, P., Konietzka, I. and Heusch, G. (2005) Regional differences of myocardial infarct development and ischemic preconditioning. Basic Res. Cardiol. 100, 48-56
9 Smith, R. M., Suleman, N., McCarthy, J. and Sack, M. N. (2002) Classic ischemic but not pharmacologic preconditioning is abrogated following genetic ablation of the TNF alpha gene. Cardiovasc. Res. 55, 553-560
10 Ran, K., Duan, K. M., Zou, D. Q., Li, Z. J., Jin, L. Y. and Chang, Y. T. (2008) Effect of isoflurane delayed preconditioning on myocardial ischemia reperfusion injury in rabbits. Zhong Nan Da Xue Xue Bao Yi Xue Ban. 33, 146-150
11 Lecour, S., Smith, R. M., Woodward, B., Opie, L. H., Rochette, L. and Sack, M. N. (2002) Identification of a novel role for sphingolipid signaling in TNF alpha and ischemic preconditioning mediated cardioprotection. J. Mol. Cell. Cardiol. 34, 509-518
12 Lecour, S., Rochette, L. and Opie, L. (2005) Free radicals trigger TNF alpha-induced cardioprotection. Cardiovasc. Res. 65, 239-243
13 El-Ani, D., Zimlichman, R., Mashiach, Y. and Shainberg, A. (2007) Adenosine and TNF-alpha exert similar inotropic effect on heart cultures, suggesting a cardioprotective mechanism against hypoxia. Life Sci. 81, 803-813
14 Dorge, H., Schulz, R., Belosjorow, S., Post, H., van de Sand, A., Konietzka, I., Frede, S., Hartung, T., Vinten-Johansen, J., Youker, K. A., Entman, M. L., Erbel, R. and Heusch, G. (2002) Coronary microembolization: The role of TNF-alpha in contractile dysfunction. J. Mol. Cell. Cardiol. 34, 51-62
15 Reil, J. C., Gilles, S., Zahler, S., Brandl, A., Drexler, H., Hultner, L., Matrisian, L. M., Welsch, U. and Becker, B. F. (2007) Insights from knock-out models concerning postischemic release of TNF alpha from isolated mouse hearts. J. Mol. Cell. Cardiol. 42, 133-141
16 Skyschally, A., Gres, P., Hoffmann, S., Haude, M., Erbel, R., Schulz, R. and Heusch, G. (2007) Bidirectional role of tumor necrosis factor-alpha in coronary microembolization—Progressive contractile dysfunction versus delayed protection against infarction. Circul. Res. 100, 140-146
17 Heusch, P., Skyschally, A., Leineweber, K., Haude, M., Erbel, R. and Heusch, G. (2007) The interaction of coronary microembolization and ischemic preconditioning: A third window of cardioprotection through TNF-alpha. Archives of Medical Science. 3, 83-92
18 Thielmann, M., Dorge, H., Martin, C., Belosjorow, S., Schwanke, U., van de Sand, A., Konietzka, I., Buchert, A., Kruger, A., Schulz, R. and Heusch, G. (2002) Myocardial dysfunction with coronary microembolization—Signal transduction through a sequence of nitric oxide, tumor necrosis factor-alpha, and sphingosine. Circul. Res. 90, 807-813
19 El-Ani, D. and Zimlichman, R. (2003) TNFalpha stimulated ATP-sensitive potassium channels and attenuated deoxyglucose and Ca uptake of H9c2 cardiomyocytes. Ann. N. Y. Acad. Sci. 1010, 716-720
20 Meldrum, D. R., Dinarello, C. A., Shames, B. D., Cleveland, J. C., Cain, B. S., Banerjee, A., Meng, X. Z. and Harken, A. H. (1998) Ischemic preconditioning decreases postischemic myocardial tumor necrosis factor-alpha production—Potential ultimate effector mechanism of preconditioning. Circulation. 98, II214-II218
21 Mubagwa, K. and Flameng, W. (2001) Adenosine, adenosine receptors and myocardial protection: an updated overview. Cardiovasc. Res. 52, 25-39
22 Hutchinson, S. A. and Scammells, P. J. (2004) A(1) adenosine receptor agonists: medicinal chemistry and therapeutic potential. Curr.Pharm.Des. 10, 2021-2039
23 Lotz, C., Liem, D. and Ping, P. P. (2011) New frontiers in myocardial protection: A systems biology approach. Journal of Cardiovascular Pharmacology and Therapeutics. 16, 285-289
24 Hinkel, R., Trenkwalder, T. and Kupatt, C. (2011) Gene therapy for ischemic heart disease. Expert Opinion on Biological Therapy. 11, 723-737
25 Marais, E., Genade, S. and Lochner, A. (2008) CREB activation and ischaemic preconditioning. Cardiovasc. Drugs Ther. 22, 3-17
26 Qu, S., Zhu, H., Wei, X., Zhang, C., Jiang, L., Liu, Y. and Xiao, X. (2010) Oxidative stress-mediated up-regulation of myocardial ischemic preconditioning up-regulated protein 1 gene expression in H9c2 cardiomyocytes is regulated by cyclic AMP-response element binding protein. Free Radic. Biol. Med. 49, 580-586.
27 Muller, B. A. and Dhalla, N. S. (2010) Mechanisms of the beneficial actions of ischemic preconditioning on subcellular remodeling in ischemic-referfused heart. Curr. Cardiol. Rev. 6, 255-264
28 Schmitt, J. P., Ahmad, F., Lorenz, K., Hein, L., Schulz, S., Asahi, M., MacLennan, D. H., Seidman, C. E., Seidman, J. G. and Lohse, M. J. (2009) Alterations of phospholamban function can exhibit cardiotoxic effects independent of excessive sarcoplasmic reticulum Ca²⁺-ATPase inhibition. Circulation. 119, 436-444
29 Cerra, M. C. and Imbrogno, S. (2012) Phospholamban and cardiac function: a comparative perspective in vertebrates. Acta Physiologica. 205, 9-25
30 Kranias, E. G. and Hajjar, R. J. (2012) Modulation of cardiac contractility by the phopholamban/SERCA2a regulatome. Circul. Res. 110, 1646-1660
31 Louch, W. E., Vangheluwe, P., Bito, V., Raeymaekers, L., Wuytack, F. and Sipido, K. R. (2012) Phospholamban ablation in hearts expressing the high affinity SERCA2b isoform normalizes global Ca²⁺ homeostasis but not Ca2+-dependent hypertrophic signaling. American Journal of Physiology-Heart and Circulatory Physiology. 302, H2574-H2582
32 MacLennan, D. H. and Kranias, E. G. (2003) Phospholamban: A crucial regulator of cardiac contractility. Nature Reviews Molecular Cell Biology. 4, 566-577
33 Kishore, R., Tkebuchava, T., Sasi, S. P., Silver, M., Gilbert, H. Y., Yoon, Y. S., Park, H. Y., Thorne, T., Losordo, D. W. and Goukassian, D. A. (2011) Tumor necrosis factor-alpha signaling via TNFR1/p55 is deleterious whereas TNFR2/p75 signaling is protective in adult infarct myocardium. Advances in TNF Family Research. 691, 433-448
34 Monden, Y., Kubota, T., Inoue, T., Tsutsumi, T., Kawano, S., Ide, T., Tsutsui, H. and Sunagawa, K. (2007) Tumor necrosis factor-alpha is toxic via receptor 1 and protective via receptor 2 in a murine model of myocardial infarction. Am. J. Physiol. Heart Circ. Physiol. 293, H743-753
35 Moe, G. W., Marin-Garcia, J., Konig, A., Goldenthal, M., Lu, X. and Feng, Q. (2004) In vivo TNF-alpha inhibition ameliorates cardiac mitochondrial dysfunction, oxidative stress, and apoptosis in experimental heart failure. Am. J. Physiol. Heart Circ. Physiol. 287, H1813-1820
36 Bao, C., Guo, J., Lin, G., Hu, M. and Hu, Z. (2008) TNFR gene-modified mesenchymal stem cells attenuate inflammation and cardiac dysfunction following MI. Scand. Cardiovasc. J. 42, 56-62
37 Kurrelmeyer, K. M., Michael, L. H., Baumgarten, G., Taffet, G. E., Peschon, J. J., Sivasubramanian, N., Entman, M. L. and Mann, D. L. (2000) Endogenous tumor necrosis factor protects the adult cardiac myocyte against ischemic-induced apoptosis in a murine model of acute myocardial infarction. Proc. Nat. Acad. Sci. U.S.A. 97, 5456-5461
38 Wang, M., Crisostomo, P. R., Markel, T. A., Wang, Y. and Meldrum, D. R. (2008) Mechanisms of sex differences in TNFR2-mediated cardioprotection. Circulation. 118, S38-45
39 El-Ani, D., Stav, H., Guetta, V., Arad, M. and Shainberg, A. (2011) Rapamycin (sirolimus) protects against hypoxic damage in primary heart cultures via Na(+)/Ca(2+) exchanger activation. Life Sci. 89.
40 EI-Ani D, Philipchik I, Stav H, Levi M, Zerbib J and Shainberg A. TNF alpha protects heart rat cultures against hypoxic damage via activation of PKA and phospholamban to prevent calcium overload. 2014. Can J Physiol Pharmacol 92(11):917-25.2014.
41 Kolk M. V., Meyberg D., Deuse T., Tang-Quan K. R., Robbins R. C., Reichenspurner H., Schrepfer S. (2009). LAD-Ligation: A Murine Model of Myocardial Infarction. JoVE. 32.

What is claimed is:

1. A composition of matter comprising a guide RNA (gRNA) sequence selected from the group consisting of SEQ ID NO: 36 and 39.

2. A mesenchymal stem cell (MSC) comprising the gRNA of claim 1.

3. A mesenchymal stem cell (MSC) having been genetically modified with a CRISPR/Cas system, wherein said CRISPR/Cas system comprises the gRNA of claim 1.

4. A method of producing mesenchymal stem cells (MSCs) having reduced level of expression of TNFR1, the method comprising contacting ex-vivo or in-vitro MSCs with a CRISPR/Cas system, wherein said CRISPR/Cas system comprises the gRNA of claim 1.

5. A pharmaceutical composition comprising the MSCs of claim 3 and a pharmaceutically acceptable carrier.

6. The pharmaceutical composition of claim 5, being formulated as a hydrated gel.

7. The pharmaceutical composition of claim 5, wherein said MSCs are cryopreserved cells.

8. The pharmaceutical composition of claim 5, wherein said MSCs are freshly isolated.

9. A method of treating an ischemic heart disease in a subject in need thereof, the method comprising administering to the subject a therapeutically effective amount of the MSCs of claim 3, thereby treating the ischemic heart disease in the subject.

10. The method of claim 9, further comprising producing said MSCs by ex-vivo or in-vitro contacting MSCs with said CRISPR/Cas system.

11. The method of claim 10, wherein said MSCs are obtained from bone marrow, peripheral blood or umbilical cord blood.

12. The method of claim 9, further comprising ex-vivo or in-vitro treating said MSCs with TNFα prior to said administering.

13. The method of claim 12, comprising cryopreserving said MSCs prior to said treating with said TNFα.

14. The method of claim 9, wherein said subject is not treated with TNFα.

15. The method of claim 9, wherein said MSCs are autologous to said subject.

16. The method of claim 9, wherein said MSCs are non-autologous to said subject.

17. The method of claim 9, wherein said MSCs are administered in a hydrated gel.

18. The method of claim 9, wherein said administering is intracardially.

19. The method of claim 9, wherein said ischemic heart disease is myocardial infarction.

20. The method of claim 9, wherein said ischemic heart disease is ischemic cardiomyopathy.
